(19) United States
(12) Patent Application Publication
(10) Pub. No.: US 2003/0083339 A1
(43) Pub. Date: May 1, 2003

(54) Compositions and methods for treating colorectal polyps and cancer

(76) Inventor: Masaaki Tamura, Nashville, TN (US)

Correspondence Address: JENKINS & WILSON, PA, 3100 TOWER BLVD, SUITE 1400, DURHAM, NC, 27707, US

(21) Appl. No.: 10/133,056

(22) Filed: Apr. 26, 2002

Related U.S. Application Data
(60) Provisional application No. 60/286,621, filed on Apr. 26, 2001.

(51) Int. Cl. A61K 31/52

(52) U.S. Cl. 514/263.4

(57) ABSTRACT

A method of decreasing a biological function of an AT₂ receptor in a subject in need thereof is disclosed. The method includes administering an effective amount of a therapeutic agent to the subject to decrease a biological function of an AT₂ receptor. Cancer therapy, particularly colorectal cancer therapy, by the method is also disclosed.

### CROSS REFERENCE TO RELATED APPLICATIONS

The present patent application is based on and claims priority to U.S. Provisional Application Serial No. 60/286,621, entitled “COMPOSITIONS AND METHODS OF TREATING COLORECTAL POLYPS AND CANCER”, which was filed Apr. 26, 2001 and is incorporated herein by reference.

### FIELD OF THE INVENTION

The present invention is generally related to treatments of colorectal polyps and cancer, and particularly related to chemoinhibition of colorectal cancer and treating colorectal cancer using antagonists of the angiotensin II (Ang II) receptor. In a preferred embodiment, antagonists of the Ang II type 2 receptor (AT₂ receptor) are employed.

### BACKGROUND OF THE INVENTION

Cancers of the colon and rectum are the fourth most commonly diagnosed cancers and rank second among cancer deaths in the United States. Individuals with Familial Adenomatous Polyposis (FAP) develop hundreds or thousands of pre-cancerous polyps throughout their colon and rectum. Left untreated, many FAP patients develop colorectal cancer in their 40's and 50's. The primary treatment for FAP is surgical removal of most or all of the colon and rectum. Clearly, this is not a desirable treatment. Hereditary nonpolyposis colon cancer syndrome (HNPCC) is another medical condition related to colorectal cancer. In this case, patients have approximately an 80% risk of developing colorectal cancer.

Angiotensin II is disclosed to stimulate cell proliferation and growth under certain conditions (Lever, et al.). Captopril, which inhibits angiotensin II synthesis, is disclosed to also inhibit cell migration and neovascularization; however, the mechanism of action is disclosed to be through an ACE-independent pathway (Volpert, et al.).

Previous research with angiotensin II analogues suggests that more than one receptor type for angiotensin II exists. U.S. Pat. No. 5,556,780 to Dzau et al., discloses that studies with nonpeptide angiotensin II receptor antagonists such as DuP 753, PD 123177 and PD 123319 led to the classification of receptor binding sites as type 1 (AT₁ receptor, which binds DuP 753, which is losartan) or type 2 (AT₂ receptor, which binds PD 123319). The '780 patent discloses that methods for identifying agents for modulating angiotensin II responsiveness can find broad utility in treating disease, including cardiovascular disease, cancer, reproductive disease, etc. The '780 patent does not disclose how the angiotensin II responsiveness should be modulated in treating disease.

The biological action of angiotensin II (Ang II) in the promotion of cellular proliferation and neovascularization is disclosed to be mediated by the AT₁ receptor (Layal, et al.; Neyses, et al., Xi, et al.; Hu, et al., Otani, et al.). On the contrary, AT₂ receptor-mediated signals are disclosed to counteract angiotensin II-AT₁ receptor mediated biological activities (Nakajima, et al., Munzenmaier, et al., Horiuchi, et al.).

Given the relative frequency of colorectal cancer in the population and the severity of surgical and chemotherapeutic treatments for the condition, it is important that new treatments be developed which specifically target colorectal neoplasms.

### SUMMARY OF THE INVENTION

In part, the present invention provides compositions and methods of treating colorectal cancer in mammals and compositions and methods of chemoinhibition of colorectal cancer in mammals.

In one aspect, the present invention provides a method of treating a colorectal cancer in a mammal in need thereof, comprising administering an effective amount of an agent to the mammal (for example, to a cell of the mammal) or to a cell of the cancer to downregulate or inhibit an AT₂ receptor gene expression or an AT₂ receptor activity.

In another aspect, the present invention provides a method of inhibiting a development of a colorectal cancer in a mammal in need thereof, comprising administering an effective amount of an agent to the mammal or to a cell of the mammal or to a cell of the cancer to downregulate or inhibit an AT₂ receptor gene expression or an AT₂ receptor activity. For example, disclosed in the present invention is a method for chemoinhibition of colorectal cancer. This is especially useful, for example, as a method of inhibiting the development of polyps or colorectal adenocarcinoma in individuals that are predisposed to their development or as a method of inhibiting a reoccurrence of polyps or colorectal carcinoma in an individual previously treated therefor.

In still another aspect, the present invention provides a method of decreasing a biological function of an AT₂ receptor in a mammal in need thereof, comprising administering an effective amount of an agent to the mammal to inhibit an AT₂ receptor gene expression or activity.

In a further aspect, the present invention provides a set of instructions delineating a treatment or a process for treating a colorectal polyp or cancer in a mammal in need thereof, comprising administering to the mammal an agent that inhibits an expression or an activity of an AT₂ receptor. The set of instructions is useful, for example, in teaching veterinarians or physicians how to care for a human or other mammal with colorectal cancer. The set of instructions is manufactured, in general, by generating text on permanent or transient media (including paper, chalkboards, and computer disks and monitors) as is known in the art, wherein the text sets forth the process.

In certain embodiments, an AT₂ receptor antagonist is provided (e.g., in a suitable container for dispensing medications) packaged together with a set of instructions for treatment of a colorectal polyp or cancer or labeled with an instruction for treating a colorectal polyp or cancer. The label, for example, can describe an amount of an AT₂ receptor inhibitor to be administered orally or anally for the treatment of a colorectal polyp or cancer.

In a still further aspect, the present invention provides a set of instructions delineating a process for chemoinhibiting a colorectal cancer in a mammal in need thereof, comprising: administering an agent to the mammal that inhibits an expression or an activity of an AT₂ receptor.

Accordingly, it is an object of the present invention to provide a novel composition and method for treating cancer, including colorectal cancer. This and other objects are achieved in whole or in part by the present invention.

An object of the invention having been stated hereinabove, other objects will be evident as the description proceeds, when taken in connection with the accompanying Drawings and Examples as best described hereinbelow.

Abbreviations

| | |
|---|---|
| ACE | angiotensin converting enzyme |
| ACEI | angiotensin converting enzyme inhibitor |
| ACF | aberrant crypt foci |
| ANG I | angiotensin I |
| ANG II | angiotensin II |
| AGT | angiotensinogen |
| ANOVA | analysis of variance |
| ANP | atrial natriutetic peptide |
| AOM | azoxymethane |
| APC | adenomatous polyposis coli |
| APP | aminopeptidase P |
| AT₁ | angiotensin II type 1 receptor |
| AT₂ | angiotensin II type 2 receptor |
| cDNA | complmentary DNA |
| CDR | complementarity determining regions |
| CYP2E1 | cytochrome P450 2E1 |
| DMEM | Dulbecco's minimal essential medium |
| DNA | deoxyribonucleic acid |
| DPP IV | dipeptidyl peptidase IV |
| ECL | enhanced chemiluminescence |
| EDTA | ethylenediaminetetraacetic acid |
| ELISA | enzyme-linked immunosorbent assay |
| FAP | familial adenomatous polyposis |
| GAPDH | glyceraldehydes phosphate dehydrogenase |
| H&E | hematoxylin and eosin |
| HAT | hypoxanthine-aminopterin-thymidine- |
| | sensitive |
| HCl | hydrochloric acid |
| HNPCC | heridtary nonpolyosis colon cancer |
| | syndrome |
| IFN | interferon |
| IP | intraperitoneal |
| KO | knockout |
| LLC | Lewis lung carcinoma |
| MAPK | mitogen activated protein kinase |
| MOI | multiplicity of infection |
| NCBI | National Center for Biotechnology |
| | Information |
| NEP | neutral endopeptidase |
| NLM | United States National Library of Medicine |
| PCR | polymerase chain reaction |
| PEG | polyethylene glycol |
| PI3-Kγ | phosphoinositide-3-OH kinase |
| RAS | renin angiotensin system |
| RIE | rat intestinal epithelial |
| RNA | ribonucleic acid |
| RT | reverse transcription |
| RT-PCR | reverse transcriptase-polymerase chain |
| | reaction |
| SDS | sodium dodecyl sulfate |
| TBS | tris-buffered saline |
| TFA | trifluoroacetic acid |

Amino Acid Abbreviations

| Single-Letter Code | Three-Letter Code | Name |
|---|---|---|
| A | Ala | Alanine |
| V | Val | Valine |
| L | Leu | Leucine |
| I | Ile | Isoleucine |
| P | Pro | Proline |
| F | Phe | Phenylalanine |
| W | Trp | Tryptophan |
| M | Met | Methionine |
| G | Gly | Glycine |
| S | Ser | Serine |
| T | Thr | Threonine |
| C | Cys | Cysteine |
| Y | Tyr | Tyrosine |
| N | Asn | Asparagine |
| Q | Gln | Glutamine |
| D | Asp | Aspartic Acid |
| E | Glu | Glutamic Acid |
| K | Lys | Lysine |
| R | Arg | Arginine |
| H | His | Histidine |

Functionally Equivalent Codons

| Amino Acid | | | Codons |
|---|---|---|---|
| Alanine | Ala | A | GCA GCC GCG GCU |
| Cysteine | Cys | C | UGC UGU |
| Aspartic Acid | Asp | D | GAG GAU |
| Glumatic acid | Glu | E | GAA GAG |
| Phenylalanine | Phe | F | UUC UUU |
| Glycine | Gly | G | GGA GGG GGG GGU |
| Histidine | His | H | GAC CAU |
| Isoleucine | Ile | I | AUA AUC AUU |
| Lysine | Lys | K | AAA AAG |
| Methionine | Met | M | AUG |
| Asparagine | Asn | N | AAC AAU |
| Proline | Pro | P | CCA CCC CCG CCU |
| Glutamine | Gln | Q | CAA CAG |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |
| Leucine | Leu | L | UUA UUG CUA CUC |
| | | | CUG CUU |
| Arginine | Arg | R | AGA AGG CGA CGC |
| | | | CGG CGU |
| Serine | Ser | S | ACG AGU UCA UCC |
| | | | UCG UCU |

### BRIEF DESCRIPTION OF THE DRAWINGS

FIGS. 1A and 1B provide verification of the targeted disruption of the murine Agtr 2 gene.

FIG. 1A is an autoradiograph depicting identification of genomic DNA of male wild type (Agtr 2^{x/y}) and hemizygous (Agtr 2^{-/y}) mice by Southern blot analysis. DNA isolated from the tails of mice was electrophoresed as described in Methods. The higher 9.5 Kb band indicates the wild type allele and the lower 6.5 Kb band corresponds to the mutant allele (Agtr^{-/y}).

FIG. 1B is a photograph of gel chromatography demonstrating tissue expression pattern of the AT₂ receptor mRNA. Total RNA was isolated from various mouse tissues, and expression of the AT₂ receptor mRNA was examined by RT-PCR. The odd-numbered lanes represent PCR products derived from the wild type mice, and the even-numbered lanes show results from the AT₂-null mice.

FIG. 2 is a bar graph showing the effect of AOM on the expression of hepatic CYP2E1 in wild type and AT₂-null mice. Mice (5 mice/group) were treated with AOM (10 mg/kg, I.P., bolus injection) and were sacrificed 0 hours (h), 6 h, or 24 h later. AT₂ receptor antagonist PD123,319 treatment (15 mg/kg/12 h, gavage administration, and 50 μg/ml in drinking tap water) was initiated 3 h prior to the AOM treatment. The liver was dissected out and the microsomal fractions were subjected to SDS-polyacrylamide gel electrophoresis followed by immunoblotting with anti-human CYP2E1 antibodies. Representative Western blots of the hepatic CYP2E1 are displayed in the upper panel. Averages of the expression levels of CYP2E1 (n=5) are displayed in the histogram. a, P 0.05 compared to the corresponding control levels in the wild type or AT₂-null mouse livers. b, P 0.05 compared to the level in the wild type mice treated with AOM for 24 h.

FIG. 3 is bar graph showing the effect of AOM on the expression of colonic O⁶-methylguanine levels in wild type and AT₂-null mice. Mice (5 mice/group) were treated with the identical procedure as described in the FIG. 2 legend. The colon was dissected out and whole DNA was extracted. O⁶-methylguanine adduct levels were determined by immuno-slot-blot analysis with a monoclonal anti-O⁶-methyldeoxyguanosine antibody. *, P 0.05 compared to the level in the wild type mice treated with AOM for 24 h. **, P 0.01 compared to the level in the wild type mice treated with AOM alone for 24 h.

FIG. 4 is an outline and schematic of a protocol for the preparation of viral particles.

FIG. 5 is a chart showing in vitro protocol for retroviral AT₂ receptor antisense delivery into target cells.

FIG. 6 is a plot of tumor volume in cubic millimeters versus days after inoculation of Lewis lung carcinoma (LLC) cells, which shows an effect of host-angiotensin II receptor status on xenografted tumor growth in mice.

### BRIEF DESCRIPTION OF THE SEQUENCES IN THE SEQUENCE LISTING

SEQ ID NO: 1 is a nucleic acid sequence encoding a human AT₂ receptor polypeptide.

SEQ ID NO: 2 is an amino acid sequence of a human AT₂ receptor polypeptide.

SEQ ID NO: 3 is a nucleic acid sequence encoding a mouse AT₂ receptor polypeptide.

SEQ ID NO: 4 is an amino acid sequence of a mouse AT₂ receptor polypeptide.

SEQ ID NO:5 is an amino acid sequence of an angiotensin I peptide.

SEQ ID NO:6 is an amino acid sequence of an angiotensin II peptide.

SEQ ID NO:7 is an amino acid sequence of a saralasin molecule.

SEQ ID NO:8 is an AT₂ receptor N-terminus fragmental peptide.

SEQ ID NO:9 is an AT₂ receptor intracellular third loop peptide.

SEQ ID NO: 10 is an N-terminus end peptide of the extramembrane section of the C-terminus of an AT₂ receptor.

SEQ ID NO: 11 is a sense AT₂ receptor-specific nucleic acid primer with added Hind III sites.

SEQ ID NO: 12 is an antisense AT₂ receptor-specific nucleic acid primer with added Hind III sites.

### DETAILED DESCRIPTION OF THE INVENTION

While the present invention is not bound to mechanism or theory in any respect, the inventor has discovered that, surprisingly, male mice with a hemizygous knockout of the AT₂ receptor gene (which is on the X chromosome) are resistant to azoxymethane (AOM)-induced colon adenocarcinoma. Treatment of mice with AOM is a model for colorectal cancer in humans. The present invention provides that angiotensin II-AT₂ receptor signaling functions as a positive regulator of biotransformation of phase I and/or phase II enzyme activities in the liver and/or colon which, in turn, regulates tumor initiation. The inventor also provides that attenuation of AT₂ receptor expression, activity, or function diminishes DNA adduct formation in colon epithelial cells. The inventor further provides that inhibition of AT₂ receptor expression, activity, or function modulates CYP1A1, CYP1A2, and/or CYP2E1 expression, activity, or function.

In certain embodiments of the present invention, the agent comprises PD123319, PD123317, or a combination of PD123319 and PD123317. PD123319 is commercially available from Sigma Chemical Company, St. Louis Mo. PD123317 is available from Parke-Davis. In certain other embodiments of the present invention, the agent comprises any biologically active composition that leads to a decrease in expression, activity, or function of the AT₂ receptor.

Humans in need of treatment or chemoinhibition for colorectal cancer include, but are not limited to: patients with colorectal polyps, patients with colorectal adenocarcinoma, patients with familial adenomatous polyposis (FAP), and patients with hereditary nonpolyposis colon cancer syndrome (HNPCC).

In certain embodiments of the present invention, the expression or activity is of a cell, tissue, colon, organ, blood, or of the mammal in general. In certain embodiments, the cell includes, but is not limited to, a colorectal tumor cell or an epithelial cell.

In certain embodiments of the present invention, an AT₂ receptor antagonist is combined with a pharmaceutically acceptable excipient, carrier, etc. and is administered by any pharmaceutically acceptable method (e.g., see U.S. Pat. No. 5,922,688 to Hung et al., incorporated herein by reference).

I. Definitions

Following long-standing patent law convention, the terms “a” and “an” mean “one or more” when used in this application, including the claims.

The term “about”, as used herein when referring to a measurable value such as an amount of activity, weight, time, dose, etc. is meant to encompass variations of ±2%, even more preferably ±1%, and still more preferably ±0.1% from the specified amount, as such variations are appropriate to perform the disclosed method.

As used herein, the term “ACE inhibitor” means an inhibitor of angiotensin converting enzyme (ACE).

As used herein, the term “agonist” means an agent that supplements or potentiates the bioactivity of a functional AT₂ receptor gene or protein, or that supplements or potentiates the bioactivity of a naturally occurring or engineered non-functional AT₂ receptor gene or protein. Alternatively, an agonist can supplement or potentiate the bioactivity of a functional gene or polypeptide encoded by a gene that is up- or down-regulated by an Ang II polypeptide and/or contains an Ang II binding site in its promoter region. An agonist can also supplement or potentiate the bioactivity of a naturally occurring or engineered non-functional gene or polypeptide encoded by a gene that is up- or down-regulated by an Ang II receptor polypeptide, and/or contains an Ang II binding site in its promoter region.

As used herein, the terms “Agtr2 gene product”, “AT₂ receptor protein”, “AT₂ receptor polypeptide”, and “AT₂ receptor peptide” are used interchangeably and mean peptides and polypeptides having amino acid sequences which are substantially identical to native amino acid sequences from an organism of interest and which are biologically active in that they comprise all or a part of the amino acid sequence of an AT₂ receptor polypeptide, or cross-react with antibodies raised against an AT₂ receptor polypeptide, or retain all or some of the biological activity (e.g., ligand binding ability) of the native amino acid sequence or protein. Such biological activity can include immunogenicity.

As used herein, the terms “Agtr2 gene product”, “AT₂ receptor protein”, “AT₂ receptor polypeptide”, and “AT₂ receptor peptide” also include analogs of an AT₂ receptor polypeptide. By “analog” is intended that a DNA or peptide sequence can contain alterations relative to the sequences disclosed herein, yet retain all or some of the biological activity of those sequences. Analogs can be derived from genomic nucleotide sequences as are disclosed herein or from other organisms, or can be created synthetically. Those skilled in the art will appreciate that other analogs, as yet undisclosed or undiscovered, can be used to design and/or construct AT₂ receptor analogs. There is no need for an “Agtr2 gene product”, “AT₂ receptor protein”, “AT₂ receptor polypeptide”, or “AT₂ receptor peptide” to comprise all or substantially all of the amino acid sequence of an AT₂ receptor polypeptide gene product. Shorter or longer sequences are anticipated to be of use in the invention; shorter sequences are herein referred to as “segments”. Thus, the terms “Agtr2 gene product”, “AT₂ receptor protein”, “AT₂ receptor polypeptide”, and “AT₂ receptor peptide” also include fusion, chimeric or recombinant AT₂ receptor polypeptides and proteins comprising sequences of the present invention. Methods of preparing such proteins are disclosed herein and are known in the art.

As used herein, the terms “Agtr2 gene” and “recombinant Agtr2 gene” mean a nucleic acid molecule comprising an open reading frame encoding an AT₂ receptor polypeptide of the present invention, including both exon and (optionally) intron sequences.

As used herein, the term “angiotensin I means a decapeptide comprising the N-terminal sequence DRVYIHPFHL (SEQ ID NO: 5).

As used herein, the term “angiotensin II” means an octapeptide, having the N-terminal sequence DRVYIHPF (SEQ ID NO: 6).

As used herein, the term “antagonist” means an agent that decreases or inhibits the bioactivity of a functional AT₂ gene or protein, or that decreases or inhibits the bioactivity of a naturally occurring or engineered non-functional AT₂ gene or protein. Alternatively, an antagonist can decrease or inhibit the bioactivity of a functional gene or polypeptide encoded by a gene that is up- or down-regulated by an Ang II polypeptide and/or contains an Ang II binding site in its promoter region. An antagonist can also decreases or inhibits the bioactivity of a naturally occurring or engineered non-functional gene or polypeptide encoded by a gene that is up- or down-regulated by an Ang II polypeptide, and/or contains an Ang II binding site in its promoter region.

As used herein, the term “AT₂ receptor” means nucleic acids encoding an angiotensin II type 2 receptor (AT₂) receptor polypeptide that can bind angiotensin II and/or one or more ligands. The term “AT₂ receptor” includes invertebrate homologs; however, preferably, AT₂ receptor nucleic acids and polypeptides are isolated from vertebrate sources. “AT₂ receptor” further includes vertebrate homologs of AT₂ receptor family members, including, but not limited to, mammalian and avian homologs. Representative mammalian homologs of AT₂ receptor family members include, but are not limited to, murine and human homologs. The term “ligand” is used broadly to refer to any agent that binds or otherwise interacts with a target.

As used herein, the term “biological activity” means any observable effect flowing from interaction between an AT₂ receptor polypeptide and a ligand. Representative, but non-limiting, examples of biological activity in the context of the present invention include association of an AT₂ receptor with a ligand, such as PD123317 and PD123319. The term “biological activity” also encompasses the both the inhibition and the induction of the expression of an AT₂ receptor polypeptide. Further, the term “biological activity” encompasses any and all effects flowing from the binding of a ligand by an AT₂ receptor polypeptide.

As used herein, the term “cancer” includes sarcomas and carcinomas. Exemplary sarcomas and carcinomas include, but are not limited to, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma (including small cell lung carcinoma and non-small cell lung carcinoma), bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenströom's macroglobulinemia, and heavy chain disease. The terms “colon cancer”, “rectal cancer”, and “colorectal cancer” are used interchangeably herein. The term cancer encompasses cancers in all forms, including polyps, neoplastic cells and preneoplastic cells.

As used herein, the terms “candidate substance” and “candidate compound” are used interchangeably and refer to a substance that is believed to interact with another moiety, for example a given ligand that is believed to interact with a complete AT₂ receptor polypeptide (or a fragment thereof), and which can be subsequently evaluated for such an interaction. Representative candidate substances or compounds include “xenobiotics”, such as drugs and other therapeutic agents, carcinogens and environmental pollutants, natural products and extracts, as well as “endobiotics”, such as steroids, fatty acids and prostaglandins. Other examples of candidate compounds that can be investigated using the methods of the present invention include, but are not restricted to, agonists and antagonists of an AT₂ receptor polypeptide, toxins and venoms, viral epitopes, hormones (e.g., opioid peptides, steroids, etc.), hormone receptors, peptides, enzymes, enzyme substrates, co-factors, lectins, sugars, oligonucleotides or nucleic acids, oligosaccharides, proteins, small molecules and monoclonal antibodies.

As used herein, the terms “cells,” “host cells” or “recombinant host cells” are used interchangeably and mean not only to the particular subject cell, but also to the progeny or potential progeny of such a cell. Because certain modifications can occur in succeeding generations due to either mutation or environmental influences, such progeny might not, in fact, be identical to the parent cell, but are still included within the scope of the term as used herein. Preferred cells include mammalian cells, and more preferably human or mouse cells.

As used herein, the terms “chimeric protein” or “fusion protein” are used interchangeably and mean a fusion of a first amino acid sequence encoding an AT₂ receptor polypeptide with a second amino acid sequence defining a polypeptide domain foreign to, and not homologous with, any domain of one of an AT₂ receptor polypeptide. A chimeric protein can present a foreign domain that is found in an organism that also expresses the first protein, or it can be an “interspecies” or “intergenic” fusion of protein structures expressed by different kinds of organisms. In general, a fusion protein can be represented by the general formula X-AT₂-Y, wherein AT₂ represents a portion of the protein which is derived from an AT₂ receptor polypeptide, and X and Y are independently absent or represent amino acid sequences which are not related to an AT₂ sequence in an organism, which includes naturally occurring mutants. The term “chimeric gene” refers to a nucleic acid construct that encodes a “chimeric protein” or “fusion protein” as defined herein.

As used herein, the term “detecting” means confirming the presence of a target entity by observing the occurrence of a detectable signal, such as a radiologic or spectroscopic signal that will appear exclusively in the presence of the target entity.

As used herein, the terms “detecting” and “detect” are used interchangeably and mean qualitative and/or quantitative determinations, including measuring an amount of enzyme activity in terms of units of activity or units activity per unit time, and the like.

As used herein, the term “DNA segment” means a DNA molecule that has been isolated free of total genomic DNA of a particular species. In a preferred embodiment, a DNA segment encoding an AT₂ receptor polypeptide refers to a DNA segment that comprises SEQ ID NOs: 1 and 3, but can optionally comprise fewer or additional nucleic acids, yet is isolated away from, or purified free from, total genomic DNA of a source species, such as Homo sapiens. Included within the term “DNA segment” are DNA segments and smaller fragments of such segments, and also recombinant vectors, including, for example, plasmids, cosmids, phages, viruses, and the like.

As used herein, the term “DNA sequence encoding a AT₂ receptor polypeptide” can refer to one or more coding sequences within a particular individual. Moreover, certain differences in nucleotide sequences can exist between individual organisms, which are called alleles. It is possible that such allelic differences might or might not result in differences in amino acid sequence of the encoded polypeptide yet still encode a protein with the same biological activity. As is well known, genes for a particular polypeptide can exist in single or multiple copies within the genome of an individual. Such duplicate genes can be identical or can have certain modifications, including nucleotide substitutions, additions or deletions, all of which still code for polypeptides having substantially the same activity.

As used herein, the terms “effective amount” and “therapeutically effective amount” are used interchangeably and mean a dosage sufficient to provide treatment for the disease state being treated. This can vary depending on the patient, the disease and the treatment being effected.

As used herein, the term “expression” generally refers to the cellular processes by which a polypeptide is produced from RNA.

As used herein, the term “gene” is used for simplicity to refer to a functional protein, polypeptide or peptide encoding unit. As will be understood by those in the art, this functional term includes both genomic sequences and cDNA sequences. Preferred embodiments of genomic and cDNA sequences are disclosed herein.

As used herein, the term “labeled” means the attachment of a moiety, capable of detection by spectroscopic, radiologic or other methods, to a probe molecule.

As used herein, the term “mammal” means humans and all domestic and wild mammals, including, without limitation, cattle, horses, swine, sheep, goats, dogs, cats, rabbits, mice, rats and the like.

As used herein, the term “mimetic” means an agonist or an antagonist to a biologically active receptor (e.g. an AT₁ receptor or an AT₂ receptor) but which has a different structural formula (primary structure) than the naturally occurring biologically active ligand for the receptor (e.g. angiotensin II). In other words, a mimetic is a non-naturally occurring biologically active ligand.

As used herein, the term “modulate” means an increase, decrease, or other alteration of any, or all, chemical and biological activities or properties of a wild-type or mutant receptor polypeptide, such as an AT₁ or an AT₂ receptor. The term “modulation” as used herein refers to both upregulation (i.e., activation or stimulation) and downregulation (i.e. inhibition or suppression) of a response.

As used herein, the term “mutation” carries its traditional connotation and means a change, inherited, naturally occurring or introduced, in a nucleic acid or polypeptide sequence, and is used in its sense as generally known to those of skill in the art.

As used herein, the term “polypeptide” means any polymer comprising any of the 20 protein amino acids, regardless of its size. Although “protein” is often used in reference to relatively large polypeptides, and “peptide” is often used in reference to small polypeptides, usage of these terms in the art overlaps and varies. The term “polypeptide” as used herein refers to peptides, polypeptides and proteins, unless otherwise noted. As used herein, the terms “protein”, “polypeptide” and “peptide” are used interchangeably herein when referring to a gene product.

The term “subject” as used herein refers to any invertebrate or vertebrate species. The methods of the present invention are particularly useful in the treatment of warm-blooded vertebrates. Thus, in a preferred embodiment, the invention concerns mammals and birds. Preferred mammals include humans and mice.

As used herein, the term “therapeutic agent” is a chemical entity intended to effectuate a change in an organism, or a combination of two or more such chemical entities. Preferably, but not necessarily, the organism is a human being. It is not necessary that a therapeutic agent be known to effectuate a change in an organism; chemical entities that are suspected, predicted or designed to effectuate a change in an organism are therefore encompassed by the term “therapeutic agent.” The effectuated change can be of any kind, observable or unobservable, and can include, for example, a change in the biological activity of a protein.

Representative therapeutic compounds include small molecules, proteins and peptides, oligonucleotides of any length, “xenobiotics”, such as drugs and other therapeutic agents, carcinogens and environmental pollutants, natural products and extracts, as well as “endobiotics”, such as epoxycholesterols. Other examples of therapeutic agents can include, but are not restricted to, agonists and antagonists of a AT₂ receptor polypeptide, toxins and venoms, viral epitopes, hormones (e.g., opioid peptides, steroids, etc.), hormone receptors, peptides, enzymes, enzyme substrates, co-factors, lectins, sugars, oligonucleotides or nucleic acids, oligosaccharides, proteins, small molecules and monoclonal antibodies.

As used herein, the term “transcription” means a cellular process involving the interaction of an RNA polymerase with a gene that directs the expression as RNA of the structural information present in the coding sequences of the gene. The process includes, but is not limited to the following steps: (a) the transcription initiation, (b) transcript elongation, (c) transcript splicing, (d) transcript capping, (e) transcript termination, (f) transcript polyadenylation, (g) nuclear export of the transcript, (h) transcript editing, and (i) stabilizing the transcript.

II. General Considerations

II.A. Human Colorectal Cancer

Colorectal cancer is still a major cause of cancer-related morbidity and mortality in the United States, even though its prognosis has improved yearly due to advances in diagnostic and surgical techniques. The American Cancer Society estimated (Landis et al., (1999) CA Cancer J Clin. 49:8-31) that 129,400 persons in the United States would develop colorectal cancer in 1999, with 56,600 deaths. It is the second leading cause of cancer death in the United States. Although the colorectal cancer incidence rates in Asia, Africa and Latin America are much lower than those in the United States (Boland, in: Textbook of Gastroenterology (2nd ed.), (Yamada, ed.), J B Lippincott, Philadelphia, Pa. (1995), pp. 1967-2026), the risk of colorectal cancer in migrant populations from these areas to the United States approaches that of United States-born Caucasians in the first generation after 20 years of residence (Potter et al., (1993) Epidemiol. Rev. 1993; 15:499-545). This and other epidemiological studies (Vogel & McPherson, (1989) Hematol. Oncol. Clin. North Am. 3:35-63; Nagao & Sugimura, (1993) Mutat. Res. 290:43-51; Sugimura et al. (1996) Environ Health Perspect. 104 Suppl 3:429-433; Potter, (1999) J. Natl. Cancer Inst. 91:916-932; Lichtenstein et al., (2000) N. Engl. J. Med. 343:78-85) indicate that this disease is etiologically related to environmental factors. Diet has been implicated as the most significant environmental factor. High-fat, low-bulk diets are a highly significant factor (Vogel & McPherson, (1989) Hematol. Oncol. Clin. North Am. 3:35-63; Nagao & Sugimura, (1993) Mutat. Res. 290:43-51; Heilbrun et al., (1989) Int. J. Cancer 44:1-6; Shankar & Lanza, (1991) Hematol. Oncol. Clin. North Am. 5:25-41; Howe et al., (1992) J. Natl. Cancer Inst. 84:1887-1896). Heavy alcohol consumption and perhaps a heavy smoking history appear to be associated with colorectal cancer (Longnecker et al., (1990) Cancer Causes Control 1:59-68. Giovannucci et al., (1994) J. Natl. Cancer Inst. 86:192-199; Giovannucci et al., (1994) J. Natl. Cancer Inst. 86:183-191). There are also significant occupational risk factors. These include exposure to asbestos, acrylonitrile, ethyl acrylate, synthetic fibers, halogens, printing materials, oils, metal fumes and dust, organic solvents, and paints and dyes (Lashner & Epstein, (1990) Int. J. Health Serv. 20:459-483; Jakobsson et al., (1994) Occup. Environ Med. 51:95-101; Stellman & Stellman, (1996) CA Cancer J Clin. 46:70-92).

In addition to the environmental factors, genetic factors also play an important role in the etiology of this disease (Boland, in: Textbook of Gastroenterology (2nd ed.), (Yamada, ed.) J B Lippincott, Philadelphia, Pa.: (1995), pp. 1967-2026; Ilyas et al., (1999) Eur. J. Cancer. 35:1986-2002; Kern & Kinzler, in: Gastrointestinal Cancer: Biology, Diagnosis and Therapy, (Rustgi, ed.) Lippincott-Raven, Philadelphia, Pa. (1995) pp. 413-422). The gene mutation that is involved in this disease includes tumor suppressor genes such as p53, mismatch repair genes, the adenomatous polyposis coli (APC) gene, the β-catenin gene and the K-ras oncogene (Ilyas et al., (1999) Eur. J. Cancer. 35:1986-2002). The etiological association of adenomatous polyps and familial adenomatous polyposis with human colorectal cancer is very well-characterized (Kern & Kinzier, in: Gastrointestinal Cancer: Biology, Diagnosis and Therapy, (Rustgi, ed.) Lippincott-Raven, Philadelphia, Pa. (1995) pp. 413-422; Allen, (1995) Semin. Surg. Oncol. 11:399-405; Giardello, Gastrointestinal Cancer: Biology, Diagnosis and Therapy, (Rustgi, ed.) Lippincott-Raven, Philadelphia, Pa. (1995), pp. 367-377). However, hereditary colorectal cancer constitutes only a small portion of the total colorectal cancer incidence (Potter, (1999) J. Natl. Cancer Inst. 91:916-932; Ilyas et al., (1999) Eur. J. Cancer. 35:1986-2002). The overall incidence of colorectal cancer is generally sporadic. Further clarification of factors that link to a genetic predisposition for colorectal cancer is very important.

II.B. Colorectal Cancer in Experimental Animals

Colon adenocarcinoma in rodents induced by the procarcinogen 1,2-dimethylhydrazine and its metabolite azoxymethane (AOM) is the most well-characterized carcinogen-induced tumor (Shamsuddin, (1986) Human Path. 17:451-453). This rodent tumor is similar to human colon adenocarcinoma in its morphology (Shamsuddin & Trump, (1981) J. Natl. Cancer Inst. 66:389-401) and in the genes that are involved in tumorigenesis (Shivapurkar et al., (1995) Cancer Lett. 96:63-70; Takahashi et al., (2000) Carcinogenesis 21:1117-1120). Mutation or dysfunction of the K-ras (Jacoby et al., (1991) J. Clin. Invest. 87:624-30) and b-catenin genes (Takahashi et al., (2000) Carcinogenesis 21:1117-1120) but not the p53 gene (Shivapurkar et al., (1995) Cancer Lett. 96:63-70) appears to be involved in the AOM-induced colon tumorigenesis in rodents. Bioactivation of AOM is reported to be catalyzed mainly by CYP2E1 (Sohn et al., (1991) Carcinogenesis 12:127-131). Because of its morphological similarity to human colon cancer, high reproducibility and relatively short latency period, this AOM-induced colon adenocarcinoma is the best-characterized animal model of human colorectal cancer.

In addition to chemical carcinogen-induced colon cancer in rodents, gene disruption of the catalytic subunits of phosphoinositide-3-OH kinase (PI3-Kγ) (Sasaki et al., (2000) Nature 406:897-902) or the guanosine-binding protein Gαᵢ₂ (Rudolph et al., (1995) Nat. Genet. 10:143-50) causes spontaneous colon cancer in rodents. These studies indicate that potential causes other than alterations in the prototypical tumor suppressor genes and oncogenes could be involved in the etiology of human colon cancer. However, the role of newly discovered potential causes involving G proteins, PI3-Kγ, etc. in human colon cancer remains yet to be clarified.

II.C. Renin-Angiotensin System and Colorectal Cancer

The renin angiotensin system (RAS) plays a key role in fluid homeostasis and in blood pressure control (Peach, (1981) Biochem. Pharmacol. 30:2745-2751). Renin, produced by the juxtaglomerular apparatus and other tissues, cleaves angiotensinogen to angiotensin 1. Angiotensin-converting enzyme (ACE) catalyzes the subsequent production of the active peptide angiotensin II (Ang II). Ang II is the most potent angiotensin metabolite and exerts a variety of biologically important actions, such as vasoconstriction, aldosterone release, and cell proliferation (Timmermans et al., (1993) Pharmacol. Rev. 45:205-251; Csikos et al., (1997) Eur. J. Endocrinol. 36:349-358; Horiuchi et al., (1999) Hypertension 33:613-621; Carey et al., (2000) Hypertension 35:155-163). These biological actions are initiated through the interaction of Ang II with specific receptors. There are at least two distinct isoforms of the receptor for Ang II (Chiu et al., (1989) Biochem. Biophys. Res. Commun. 15:196-203; Gallinat et al., (2000) Am. J. Physiol. Endocrinol. Metab. 278:E357-74). Both receptor signals are mediated through G-protein coupling, and they are classified as G-protein-coupled seven transmembrane receptors (Timmermans et al., (1993) Pharmacol. Rev. 45:205-251; Whitebread et al., (1989) Biochem. Biophys. Res. Commun. 163:284-291).

The major isoform, AT₁, is distributed in a wide variety of tissues and is mainly responsible for most of the known biological actions elicited by Ang II (Timmermans et al., (1993) Pharmacol. Rev. 45:205-251; Csikos et al., (1997) Eur. J. Endocrinol. 36:349-358). The second receptor subtype (AT₂) was cloned in 1993 (Kambayashi et al., (1993) J. Biol. Chem. 268:24543-24546; Mukoyama et al., (1993) J. Biol. Chem. 268:24539-24542) and was shown to be expressed abundantly in the mesenchyme of the fetus only in the late gestation period (Grady et al., (1991) J. Clin. Invest. 88:921-933). In adults the AT₂ receptor is expressed in a variety of tissues in a relatively smaller quantity than the AT₁ receptor. AT₂ receptor-mediated Ang II actions are diverse (Scheuer & Perrone, (1993) Am. J. Physiol. 264:R917-R923; Lokuta et al., (1994) J. Biol. Chem. 269:4832-4838; Ichiki et al., (1995) Nature 377:748-750; Nakajima et al., (1995) Proc. Natl. Acad. Sci. U.S.A. 92:10663-10667; Stoll et al., (1995) J. Clin. Invest. 95:651-657; Siragy & Carey, (1996) J. Clin. Invest. 97:1978-1982; Dimmeler et al., (1997) Circ. Res. 81:970-976; Jin et al., (1998) Am. J. Physiol. 275:R515-R523; Zhu et al., (1998) J. Neurosci. 18:679-686), and recent studies suggest that protein tyrosine and serine/threonine phosphatase activation, nitric oxide/cGMP production and arachidonic acid/prostaglandins production are involved in the mechanism of AT₂ receptor-mediated biological reactions (Csikos et al., (1997) Eur. J. Endocrinol. 36:349-358; Horiuchi et al., (1999) Hypertension 33:613-621; Carey et al., (2000) Hypertension 35:155-163; Gallinat et al., (2000) Am. J. Physiol. Endocrinol. Metab. 278:E357-74). In addition, the AT₂ receptor mediates apoptosis in a few types of cells in vitro (Csikos et al., (1997) Eur. J. Endocrinol. 36:349-358; Horiuchi et al., (1999) Hypertension 33:613-621; Carey et al., (2000) Hypertension 35:155-163; Gallinat et al., (2000) Am. J. Physiol. Endocrinol. Metab. 278:E357-74; Yamada et al., (1996) Proc. Natl. Acad. Sci. U.S.A. 93:156-60). Although Ang II is a well-known stimulus of CYP11B2 expression in adrenal glomerulosa cells (Lyall et al., (1992) J. Hypertens. 10:1463-1469), little information is available on its involvement in CYP regulation in other tissues.

In relation to cancer, Ang II induces expression of protooncogenes, such as c-fos and c-myc, and promotes cell proliferation and growth (Lyall et al., (1992) J. Hypertens. 10:1463-1469; Neyses et al., (1993) J. Hypertens. 11:927-934; Sadoshima & Izumo, (1993) Circ. Res. 73:424-438). Ang II also stimulates neovascularization, which is a requirement for solid tumor growth (Fernandez et al., (1985) J. Lab. Clin. Med. 105:141-145; le Noble et al., (1996) J. Vasc. Res. 33:480-488). In addition to Ang II, in vitro studies demonstrate that ACE inhibitors retard growth of cultured cancer cells (Chen et al., (1991) Proc. Soc. Exp. Biol. Med. 196:280-283; Reddy et al., (1995) Proc. Soc. Exp. Biol. Med. 210:221-226). ACE inhibitors also inhibit angiogenesis and growth of xenografts in rats (Volpert et al., (1996) J. Clin. Invest. 98:671-679). Recent reports suggest that long term usage of ACE inhibitors reduces risks of several human cancers, including colorectal cancer (Lever et al., (1998) Lancet 352:179-184). Furthermore, several reports suggest that plasma ACE activity can be a tumor biomarker (Varela & Bosco Lopez Saez, (1993) Oncology 50:430-435; Kardum et al., (1999) Eur. J. Gastroenterol. Hepatol. 11:1209-1213). Therefore, the renin angiotensin system is an important component in not only cardiovascular diseases but also in cancer, although the mechanism by which the ACE inhibitor reduces the risk of cancer is not clear.

II.D. Angiotensin Converting Enzyme

Angiotensin-converting enzyme (ACE) catalyzes the cleavage of angiotensin I into angiotensin Ii, which has an activity of raising blood pressure. ACE and neutral endopeptidase (NEP) catalyze the degradation of bradykinin and substance P into inactive metabolites. NEP also catalyzes the degradation of atrial natriutetic peptide (ANP) into inactive metabolites. In contrast to angiotensin II, bradykinin and ANP have an activity of lowering blood pressure. Therefore, the use or administration of an ACE/vasopeptidase inhibitor generally results in a reduction in blood pressure because these inhibitors reduce angiotensin II production and increase bradykinin and/or ANP concentrations by inhibiting their degradation into inactive metabolites. Included in the many additional applications of ACE inhibitors are the treatment of cardiac diseases, renal diseases, and diabetes. Vasopeptidase inhibitors are also under investigation for use in these conditions and are awaiting regulatory approval. The clinical effectiveness of these inhibitors might result from influences on multiple physiological pathways, however, and the present invention is in no way bound by theory or mechanism.

The ACE enzymatic pathway is the primary pathway for angiotensin II formation and bradykinin degradation. Alternative pathways have been identified for the degradation of both bradykinin and substance P, however. These pathways comprise the degradation of bradykinin by the aminopeptidase P (APP) and dipeptidyl peptidase IV (DPP IV) enzymes, and the degradation of substance P by DPP IV. In general, the contribution of the alternative DPP IV and APP pathways could, but not necessarily, increase during ACE/vasopeptidase inhibition for individuals that are at a reduced risk of angioedema (“non-ACEI”) even in comparison to normotensives. On the other hand, individuals with increased angioedema risk (“ACEI-associated”) show a reduction alternative pathway activity (for example, DPP IV).

II. E. Angiotensin II Receptor Antagonists and ACE Inhibitors

An ACE inhibitor derived from food protein was first reported in 1979 by Oshima et al. (Oshima et al., (1979) Biochim. Biophys. Acta 556:128). Since then over 40 ACE inhibitory peptides have been disclosed to date (see, e.g., Ariyoshi, (1993) Trends Food Sci. Tech., May, 1993, p. 139). A number of ACE inhibitory peptides have been derived from foodstuff such as sour milk (Nakamura et al., (1995) J. Dairy Sci. 78: 777), tuna tissue (Kohama et al., (1988) Biochem. Biophys. Res. Comm. 155(1): 332), sardine muscle (Matsuda et al., (1992) Nippon Nogeigaku Kaishi 66(11): 1645), oyster protein (Matsumoto et al., (1994) Nippon Shokuhin Kogyo Gakkaishi 41(9): 589), Ficus carica (Maruyama et al., (1989) Agric. Biol. Chem. 53(10): 2763), and rice (Muramoto & Kawamora, (1991) Food Ind. 34(11): 18). Furthermore, numerous patent applications have been filed in relation with ACE inhibitory peptides, including synthesized inhibitors as well as those isolated from natural products. See e.g., U.S. Pat. Nos. 5,449,661; 5,071,955; 4,692,459; 4,585,758; 4,512,979; 4,191,753; 3,832,337; and European Patent No. EP174162.

II.E.1 Receptor Antagonists

Recently, several nonpeptidic Ang II receptor antagonists have been developed. Losartan, the first AT₁ receptor-specific antagonist developed, is already in clinical use. The IC₅₀ of losartan is 19 nM for the inhibition of ligand-receptor binding, and no serious side effects have been reported in the daily administration of the pressor dose (10-50 mg/kg/day) in both man and animals (Timmermans et al., (1993) Pharmacol. Rev. 45:205-251). PD123319, an AT₂ receptor-specific antagonist, is also commercially available only for laboratory use. The IC₅₀ of PD123319 is 10 nM for the inhibition of ligand-receptor binding (Dudley et al., (1991) Mol. Pharmacol. 40:360-367). Chronic administration of PD123319 (30 mg/kg/day) does not show any adverse effects in rats (Levy et al., (1996) J. Clin. Invest. 98:418-425).

In some aspects of the present invention, AT₂ receptor antagonists are employed. In one aspect of the present invention, an AT₂ receptor antagonist is employed to treat a cancer. In another aspect, an AT₂ receptor antagonist is employed to prevent a cancer. And in yet another aspect of the present invention, an AT₂ receptor antagonist is employed to decrease a biological function of an AT₂ receptor.

Several AT₂ receptor antagonists are known and are commercially available. For example, the compounds PD 123317, PD 123319, Saralasin, and CGP 42112A are known AT₂ receptor antagonists. The compounds PD 123319 is commercially available from Sigma Chemical Co. of St. Louis, Mo., and the compound PD123317 is a Parke Davis test compound.

Saralasin is a short peptide comprising the sequence Sar-Arg-Val-Tyr-Val-His-Pro-Ala (SEQ ID NO: 7). The preparation of saralasin has been described (U.S. Pat. No. 3,751,404 to Sipos et al.) Losartan is a non-peptide AT₁ receptor antagonist. The chemical structure of Losartan is

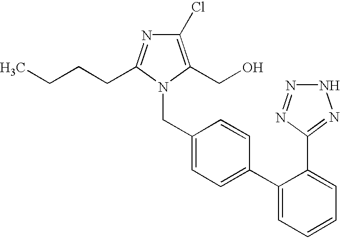

The preparation of Losartan is described in U.S. Pat. No. 5,138,069 to Carini et al.

Other representative Ang II receptor antagonists include candesartan cilexetil, eprosartan, irbesartan, tasosartan, telmisartan, valsartan, BMS-184699, 3-(2′-(tetrazol-5-yl)-1,1′-biphen-4-yl)methyl-5,7-dimethyl-2-ethyl-3H-imidazo[4,5-b]pyridine, BAY 106734, BIBR363, CL329167, E4177, EMD73495, HN65021, HR720, HOE720, LRB081, SC52459, SL910102, UP2696, YM358, EMD66397, ME3221, TAK536, BMS 184698, CGP42112A, CGP49870, CP14R13O, E4188, EMD666R4, EXP9954, FRI 153332, GA0050, KT3579, LF70156, LRB057, LY266099, LY301875, PD 123177, PD 126055, SC51757, SC54629, U96849, UK77778, WAY126227, WK1260, WK1492, YH1498, and YM31472.

See also U.S. Pat. Nos. 4,333,943, 4,880,804, 5,053,329, and European Patents 0 245 637, 0 253 310, and 0 291 969, and Wong et al., Hypertension 15:459 (1990), Wong et al., (1990) J. Pharmacol. Exp. Ther. 256:211 and Chiu et al., (1989) Biochem. Biophys. Res. Comm. 165:196-203.

II.E.2. ACE Inhibitors

ACE inhibitors have been clinically utilized for the last two decades for blood pressure regulation, and long term usage of the pressor dose (35-75 mg/kg/day) has been approved. The most frequent side effect is coughing, which is exhibited by approximately 10% of patients (Goldszer et al., (1988) Am. J. Med. 85:887). Among many ACE inhibitors, captopril is the first ACE inhibitor developed and is still used clinically. The IC₅₀ of captopril for ACE inhibition is approximately 10 nM (Johnston et al., (1986) J. Cardiovasc. Pharmacol. 8:S9-S14). Captopril contains a free thiol group and is an effective radical scavenger and antioxidant (Migdalof et al., (1984) Drug Metab. Rev. 15:841-869).

ACE inhibitors can differ in the chemical structure of their active moieties, in potency, in bioavailability, in plasma half-life, in route of elimination, in their distribution and affinity for tissue-bound ACE, and in whether they are administered as prodrugs. The same can be true for vasopeptidase inhibitors. Those of ordinary skill in the art recognize that the side effects of ACE inhibitors can be divided into those that are class specific and those that relate to specific agents. ACE inhibitors decrease systemic vascular resistance without increasing heart rate and they promote natriuresis. ACE inhibitors have proved effective in the treatment of hypertension. ACE inhibitors also decrease mortality in congestive heart failure and left ventricular dysfunction after myocardial infarction, and they delay the progression of diabetic nephropathy.

Certain examples of known and commercially available ACE inhibitors are listed in Table 1. This is not meant to be an exhaustive list, but merely exemplary of certain ACE inhibitors that can be employed in treating subjects in need of treatment therewith. An example of a vasopeptidase inhibitor in development includes omapatrilat (brand name VANLEV™ by Bristol-Meyers Squibb).

TABLE 1
Marketed ACE Inhibitors

| | | Company |
|---|---|---|
| Compound Name | | (Maker of Brand |
| (Generic Drug) | Brand Name | Name) |
| Captopril | CAPOTEN | |
| Enalapril | VASOTEC | Merck |
| Lisinopril | ZESTRIL | Zeneca |
| Lisinopril | PRINIVIL | Merck |
| Benazepril | LOTENSIN | Novartis |
| Quinapril | ACCUPRIL | Parke-Davis |
| Ramipril | ALTACE | Monarch |
| Trandolapril | MAVIK | Knoll (Roussel Uclaf) |
| Moexipril | UNIVASE | Schwartz |
| Fosinopril | MONOPRIL | BMS |
| Perindep | ACESRI | Solva |

As noted, ACE converts angiotensin I to angiotensin II. Angiotensin II increases blood pressure and is considered a main cause of essential hypertension. A variety of studies have been directed to substances inhibiting ACE actions, primarily addressing the suppression of a rise in blood pressure.

Therapeutic vasodepressors such as CAPTOPRIL™ and D-2-methyl-3-mercaptopropanoyl-L-proline have been synthesized as ACE inhibitors. Additional ACE inhibitors available commercially include ENALAPRIL™, ENALAPRILAT™, QUINAPRIL™, RAMIPRIL™, CILAZAPRIL™, DELAPRIL™, FOSENOPRIL™, ZOFENOPRIL™, INDOLAPRIL™, LISINOPRIL™, PERINDOPRIL™, SPIRAPRIL™, PENTOPRIL™, PIVOPRIL™, and known pharmaceutically acceptable salts thereof. Several of these ACE inhibitors are presented in Table 1. From foodstuff, peptides having ACE inhibiting activities have been separated through enzymatic hydrolysis of casein (Japanese Laid-Open Patent Publication Nos. 62-270533, 64-5497, 64-83096) and soybean protein (Japanese Laid-Open Patent Publication Nos. 3-1671981).

II.F. Regulation of Angiotensin II AT₂ Receptor Expression

AT₂ receptor expression is upregulated by increases in intracellular sodium and calcium (Tamura et al., (1999) Hypertension 33:626-632). AT₂ receptor expression is downregulated by lipopolysaccharides and proinflammatory cytokines through nitric oxide and cGMP production (Tamura et al., (1999) Eur. J. Pharmacol. 386:289-295). In vivo AT₂ receptor induction in rats, which were raised on a purified synthetic diet (Tamura et al., (2000) Can. J. Physiol. Pharmacol. 78:548-56), was also studied. This study revealed that prostaglandins are invoved in post-receptor signaling. AT₂-knockout mice do not respond to pressure overload with cardiac hypertrophy (Senbonmatsu et al., (2000) J. Clin Invest. 106:R1-5). The deficiency in AT₂-knockout mice appears to be associated with absence of a response in the p70^{S6} kinase signaling cascade, which is essential for a hypertrophic response.

II.G. Cytochrome P450 Monooxygenases and Colorectal Cancer

Carcinogenic xenobiotics in the environment can be activated or detoxified by phase I and phase II biotransformation enzymes, including cytochrome P450s, glucuronosyltransferases, glutathione S-transferases and others. Guengerich, (1990) Crit. Rev. Biochem. Mol. Biol. 25:97-153; Guengerich et al., (1991) Chem. Res. Toxicol. 4:168-179; Armstrong, (1987) CRC Crit. Rev. Biochem. 22:39-88; Bock, (1991) Crit. Rev. Biochem. Mol. Biol. 26:129-150. Metabolism mainly occurs in the liver, but also in the gastrointestinal tract. Guengerich, (1990) Crit. Rev. Biochem. Mol. Biol. 25:97-153; Rosenberg & Mankowski, (1994) Carcinogenesis 15:73-78; Grove et al., (1997) J. Biol. Chem. 272:1621-1627; Strassburg et al., (1999) Gastroenterology 116:149-160. Cytochrome P450s are a multi-gene super family of heme-containing enzymes that catalyze the oxidative metabolism of many compounds. Guengerich, (1990) Crit. Rev. Biochem. Mol. Biol. 25:97-153; Guengerich et al., (1991) Chem. Res. Toxicol. 4:168-179; Armstrong, (1987) CRC Crit. Rev. Biochem. 22:39-88. Their substrates are a wide variety of endogenous compounds as well as many xenobiotics, such as carcinogens, mutagens, and drugs. Guengerich, (1990) Crit. Rev. Biochem. Mol. Biol. 25:97-153; Guengerich et al., (1991) Chem. Res. Toxicol. 4:168-179; Armstrong, (1987) CRC Crit. Rev. Biochem. 22:39-88. The main cytochrome P450s participating in the metabolism of xenobiotics are Families 1, 2, and 3.

CYP1A1, 1A2 and 2E1 are of particular interest because they are involved in the metabolic activation of xenobiotics associated with colorectal cancer. The CYP1A1 gene encodes the aryl hydrocarbon hydroxylase enzyme responsible for the activation of polycyclic aromatic hydrocarbons, putative carcinogens mainly derived from cigarette smoke and smoked or barbecued foods. Badawi et al., (1996) Prog. Clin. Biol. Res. 395:109-140. CYP1A2 is involved in the metabolic activation of heterocyclic amines, carcinogens generated by cooking meat for prolonged periods at a high temperature. Lang et al., (1994) Cancer Epidemiol. Biomarker. Prev. 3:675-682. CYP2E1 is an ethanol-inducible enzyme and is known to be involved in the activation of many low molecular weight organic chemicals, including N-nitrosoamines, present in tobacco smoke and also derived from the diet. Koop & Tierney, (1990) Bioessays 12:429-435; Yang et al., (1990) Drug Metab. Rev. 22:147-159; Guengerich & Shimada, (1991) Chem. Res. Toxicol. 4:391-407. There are several restriction fragment length polymorphisms (RFLPs) in CYP1A1, 1A2 and 2E1. Koop & Tierney, (1990) Bioessays 12:429-435. Kawajiri et al., (1990) FEBS Lett. 263:131-3; Hayashi et al., (1991) J. Biochem. (Tokyo) 110:407-11; Chida et al., (1999) Jpn. J. Cancer Res. 90:899-902; Hayashi et al., (1991) J. Biochem. (Tokyo) 110:559-565; Watanabe et al., (1994) J. Biochem. (Tokyo) 116:321-6. These genomic polymorphisms in CYP1A1 and 2E1 have been suggested to be associated with the risks of lung cancer (Kawajiri et al., (1990) FEBS Lett. 263:131-3; Hayashi et al., (1991) J. Biochem. (Tokyo) 110:407-11) and colorectal cancer (Kiss et al., (2000) Anticancer Res. 20:519-522; Sivaraman et al., (1994) Cancer Res. 54:3692-3695; Uematsu et al., (1992) Tohoku J. Exp. Med. 168:113-117. A rapid CYP1A2 phenotype has been implicated in association with the risk for human colorectal cancer. Lang et al., (1994) Cancer Epidemiol. Biomarker Prev. 3:675-682.

Thus, an aspect of the present invention is that the Ang II-AT₂ receptor-mediated signal is involved in AOM-induced tumorigenesis in the colon. Another aspect of the present invention pertains to the evaluation of an endogenous upstream regulation mechanism of hepatic cytochrome P450 protein expression by the Ang II receptor-mediated signals. Procedures in which two kinds of Ang II receptor gene-disrupted (AT₁ₐ-KO and AT₂-KO) mice and the hepatocytes derived from them are used in this evaluation.

In another aspect of the present invention, regulation of cytochrome P450-dependent bioactivation of procarcinogens through pharmacological and genetic regulation of the upstream mechanism is relevant to chemoprevention methods. Since no harmful side effects have been noted in long-term administration of AT₂ receptor antagonists (Levy et al., (1996) J. Clin. Invest. 98:418-425) or angiotensin-converting enzyme inhibitors, regulation of cytochrome P450s that are potentially involved in human colon tumorigenesis through AT₂ receptor modulation is of interest in clinical applications.

In yet another aspect, the present invention pertains to a method to regulate AT₂ receptor function through genetic intervention with viral AT₂ receptor antisense cDNA. This procedure can be more practical for cancer prevention, since viral delivery of antisense cDNA should attenuate only the target gene for a long time. Combinations of chemotherapy of colorectal cancer by pharmacological regulation of the AT₂ receptor and genetic intervention with AT₂ receptor antisense cDNA are also employed. The construct can be a viral vector or a non-viral vector (e.g. plasmids, cosmids). Suitable viral vectors include adenoviruses, adeno-associated viruses (AAVs), retroviruses, pseudotyped retroviruses, herpes viruses, vaccinia viruses, Semiliki forest virus, and baculoviruses.

III. Peptide, Polypeptide and Polynucleotide Components of the Present Invention

A variety of biological information including nucleotide and peptide sequence information is available from public databases provided, for example, by the National Center for Biotechnology Information (NCBI) located at the United States National Library of Medicine (NLM). The NCBI is located on the world wide web at the URL “http://www.ncbi.nlm.nih.gov/” and the NLM is located on the world wide web at the URL “http://www.nim.nih.gov/”. The NCBI website provides access to a number of scientific database resources including: GenBank, PubMed, Genomes, LocusLink, Online Mendelian Inheritance in Man (OMIM), Proteins, and Structures. A common interface to the polypeptide and polynucleotide databases is referred to as Entrez which can be accessed from the NCBI website on the World Wide Web at URL “http://www.ncbi.nlm.nih.gov/Entrez/” or through the LocusLink website.

The following subsections disclose a plurality of molecules that can form an element of the present invention. This discussion is not meant to be an inclusive list of molecules that can form a component of the present invention. The following subsections are included to provide additional detail regarding components of the present invention, as well as to help illustrate how the various molecules relate to one another in vivo.

III.A. Angiotensin I and Angiotensin II

The following summary of angiotensinogen is available in the NCBI LocusLink database:

The human AGT gene product, pre-angiotensinogen, is expressed in the liver and is cleaved by the enzyme renin in response to lowered blood pressure. The resulting product, angiotensin I is then cleaved by angiotensin converting enzyme (ACE) to generate the physiologically active enzyme [sic, peptide] angiotensin II. Human pre-angiotensinogen is encoded by two mRNAs that differ only in the length of the 3′-untranslated region due to postulated use of two polyadenylation sites. There may also be alternative initiation codons (nucleotides 40-42 and 67-69). AGT is involved in maintaining blood pressure and in the pathogenesis of essential hypertension and preeclampsia.

The Homo sapiens Official Gene Symbol and Name is: AGT: angiotensinogen.

Angiotensin II is a polypeptide having, in man, the amino acid sequence Asp-Arg-Val-Tyr-Ile-His-Pro-Phe. (SEQ ID NO: 6) (The abbreviations used herein are those published by the IUPAC-IUB Commission on Biochemical Nomenclature, Archives Biochem. Biophys. 150:1 (1972). The sequence is read N-terminus to carboxyl terminus. Unless otherwise indicated, the L stereochemical configuration is intended.) Some variation of this sequence, particularly at the fifth amino acid, can occur in lower animals. Angiotensin II (or Ang II) is a pressor substance formed from a decapeptide, angiotensin I, by the action of angiotensin converting enzyme (ACE). Angiotensin II is believed to exert its effect by interaction with a receptor. An angiotensin II antagonist, also known as an angiotensin II receptor blocker, prevents angiotensin II from exerting its effect, presumably by preventing interaction of angiotensin II with its receptor site.

The hormone angiotensin II is recognized as one of the most potent vasopressor agents that produces hypertension in mammals. The action of the enzyme renin on the plasma protein substrate angiotensinogen results in the production of an inactive decapeptide, angiotensin 1, which upon conversion by the non-selective angiotensin converting enzyme (ACE) provides angiotensin II, the active hormone. See, e.g., Regoli et al., (1974) Pharm. Rev. 26: 69.

Angiotensin II causes vasoconstriction and stimulates aldosterone secretion (from the adrenal gland) that results in a rise of both blood volume and pressure. Inhibitors of angiotensin II are therefore useful in treating hypertension, congestive heart failure, renal insufficiency associated with diabetic or hypertensive nephropathy, and glaucoma. See, e.g., Garrison et al., in The Pharmacological Basis of Therapeutics, 8th Edition, (Gilman, Goodman, Rall, Nies, and Taylor, eds), Pergamon Press, New York, 1990: p. 761-762; and Dzau, (1991) New Engl. J. Med. 324: 1124-1130.

Angiotensin II also can act on other organs such as the brain (Fitzsimmons, (1980) Rev. Physiol. Biochem. Pharmacol. 87:117). Antagonists of angiotensin II are therefore useful in enhancing cognitive performance in patients affected by conditions such as age associated mental impairment or Alzheimer's disease, and in treating cognitive disorders such as anxiety. See, e.g., Dennes et al., (1992) Brit. J. Pharmacol. 105: 88; and Barnes et al., (1991) FASEB J., 5: 678.

In addition, angiotensin II acts on a variety of glandular tissues including the kidney, liver, and ovaries. Antagonists of angiotensin II are useful in treating conditions, disorders, or diseases of these tissues associated with excessive or unregulated angiotensin II activity. Antagonists of angiotensin II are also useful in treating kidney damage due to non-steroidal antiinflammatory agents.

Angiotensin II has a role in regulation of the rate of cell growth and differentiation. Inhibitors of angiotensin II are therefore useful in treating disorders marked by excessive cell proliferation such as restenosis. See, e.g., Naftilan et al., (1989) J. Clin. Invest. 83: 1419, Kauffman et al., (1991) Life Sci. 49: 223-228, and Jackson et al., (1988) Nature 335: 437. Angiotensin II is formed in the human body through proteolysis of angiotensin I (Ang I) primarily through the action of angiotensin-converting enzyme.

III.B. Angiotensin II Receptor

In mammalian cells, Ang II mediates its effects via at least two plasma membrane receptors: AT₁ and AT₂ receptors. Both receptor subtypes have been cloned and pharmacologically characterized (Kambayashi Y. et al., J.B.C. 268:24543-24546,1993, Mukoyama, M et al., J.B.C. 268:24539-24542, 1993, Murphy, T J, et al. Nature, 351:233-236, 1991, Sasaki, K et al. Nature, 351:230-233, 1991). The Ang II receptors can be distinguished according to inhibition by specific antagonists, AT₁ receptors are selectively antagonized by biphenylimidazoles, such as losartan, whereas tetrahydroimidazopyridines specifically inhibit AT₂ receptors (Ardaillou, R. J. Am. Soc. Nephrol., 10:S30-S39, 1999). The AT₂ receptor may also be selectively activated by CGP-42112A. This is a hexapeptide analog of Ang II, which may also inhibit the AT₂ receptor, depending on concentration (Criscione, L. et al., J. Cardiovasc. Pharmacol. 16 Suppl 4:S56-S59, 1990). Two other angiotensin receptors have been described: AT₃ and AT₄ subtypes (Berk, BC et al. Circ. Res., 80:607-616, 1997, Chaki, S. Hypertension, 20:397397, 1992, Swanson, GN et al. Regul. Pept., 40:409-419, 1992). However, the pharmacology of AT₃ and AT₄ receptors has not been fully characterized, and therefore these receptors are not included in a definitive classification of mammalian angiotensin receptors as defined by the International Union of Pharmacology Nomenclature Subcommittee for Angiotensin Receptors (De Gasparo, M., et al. Pharmacol. Rev. 52:415-472, 2000, De Gasparo, M., et al. Hypertension 25:924-927, 1995).

AT₁ Receptor

The AT₁ receptor belongs to the seven-membrane-spanning G protein-coupled receptor family and typically activates phospholipase C (PLC) through the heterotrimeric G_{q} protein, although it may also signal through Gₗ, G_{11/13} and Gₛ (Berk, BC et al. Circ. Res., 80:607-616, 1997, De Gasparo, M., et al. Hypertension 25:924-927,1995, Mukoyama, M et al., J.B.C. 268:24539-24542, 1993, Murphy, T J, et al. Nature, 351:233-236, 1991, Sasaki, K et al. Nature, 351:230-233, 1991). The human AT₁ receptor gene is mapped to chromosome 3 (Curnow, K M., et al., Mol. Endocrinol. 6:1113-1118, 1992). AT₁ receptors are widely distributed throughout the cardiovascular, renal endocrine, and nervous system in humans (Allen, A M., et al. Am. J. Hypertens., 13:31S-38S, 1999). In rodents, the AT₁ receptor has two functionally distinct subtypes, AT_{1A} and AT_{1B}, with >95% amino acid sequence homology (Ichiki, T, et al., Nature 377:748-750, 1995, Iwai, N. and Inagami, T., FEBS Lett., 298:257-260, 1992). On the basis of the cDNA sequence, the AT₁ receptor is composed of 359 amino acids (Sandberg, K. Trends Endocrinol. Metab., 5:28-35, 1994). It is a glycoprotein and contains extracellular glycosylation sites at the amino terminus (Asn⁴) and the second extracelluar loop (Asp¹⁷⁶ and Asn¹⁸⁸) (Desarnaud, F., et al. Biochem. J. 289:289-297, 1993). The transmembrane domain at the amino-terminal extension and segments in the first and third extracellular loops are responsible for G protein interactions with the receptors involves receptor phosporylation, which may be mediated, in part, via caveola (Hunyady, L. J. Am. Soc. Nephrol. 10:S47-S56, 2001).

AT₂ Receptor

The second major angiotensin receptor isoform is the AT₂ receptor. The gene of this receptor is localized as a single copy on the X chromosome (Lazard, D. et al. Receptors Channels 2:271-280, 1994). The AT₂ receptor is a seven-transmembrane-type, G protein-coupled receptor comprising 363 amino acids. It has low amino acid sequence homology (˜34%) with AT_{1A} or AT_{1B} receptors (Inagami, T., et al. J. Hypertens 10:713-716,1992, Mukoyama, M et al., J.B.C. 268:24539-24542, 1993). Although the exact signaling pathways and the functional roles of AT₂ receptors are unclear, these receptors may antagonized, under physiological conditions, AT₁-mediated actions (Ciuffo, G M., et al., Regul. Pept. 74:129-135,1998, Zhuo, J L., et al., J. Hypertens, 16:2027-2037, 1998) by inhibiting cell growth and by inducing apoptosis and vasodilation (Gallinat, S., et al., Am. J. Physiol. Endocrinol. Metab. 278:E357-E374, 2000, Hayashida, W., et al., Hypertension 28:75, 1996, Horiuchi, M., Et al., J. Biol. Chem. 22:19022-19026, 1997, Horiuchi, M., et al., Trends Endocrinol. Metab. 10:391-396, 1999, Siragy, H M., et al. Hypertension 35:1074-1077, 2000, Unger, T. J. Hypertens, 17:1775-1786, 1999).

The AT₂ receptor is ubiquitously expressed in human fetal mesenchymal tissues. However, the expression of this receptor rapidly declines after birth (Nahmias, C and Strosberg, A D., Trends Pharmacol. Sci 46:223-225, 1995). In adults, AT₂ receptor expression is detectable in the pancreas, heart, kidney, adrenals, brain, and vasculature (Lazard, D., et al., Eur. J. Biochem, 220:919-926, 1994, Ozono, R., et al., Hypertension 30:1238-1246, 1997, Touyz, R M., et al., Hypertension 33:366-372, 1999, Touyz R M., and Schiffrin E L., Hypertension 30:222-229,1997, Viswanathan, M. and Saavedra, J M., FASEB J. 6: A1013, 1992, Wang Z Q., et al., Hypertension 34:108,1999, Wang Z Q. et al., Hypertension 34:108,1999).

The expression of both angiotensin receptor types is tightly regulated. The AT₁ receptor my be subject to “negative feedback” by Ang II (Aguilera, G. and Catt, K., Circ Res. 49:751-758, 1981), Whereas expression of the AT₂ receptor is upregulated by sodium depletion (Ozono, R., et al., Hypertension 30:1238-1246, 1997) and is inhibited by Ang II and growth factors such as PDGF and EGF (Ichiki, T., et al. Circ. Res. 77:1070-11076,1995).

IV. Therapeutic Methods

IV.A. Subjects

The methods of the present invention can be useful for treatment of a subject, as defined herein. The subject treated in the present invention in its many embodiments is preferably a human subject, although it is to be understood that the principles of the invention indicate that the invention is effective with respect to all vertebrate species, including mammals, which are intended to be included in the term “subject”. In this context, a mammal is understood to include any mammalian species in which treatment is desirable, particularly agricultural and domestic mammalian species.

The term “subject” as used herein refers to any invertebrate or vertebrate species. The methods of the present invention are particularly useful in the treatment of warm-blooded vertebrates. Thus, the invention concerns mammals and birds. More particularly, provided is the treatment and/or diagnosis of mammals such as humans, as well as those mammals of importance due to being endangered (such as Siberian tigers), of economical importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos, as well as fowl, and more particularly domesticated fowl, e.g., poultry, such as turkeys, chickens, ducks, geese, guinea fowl, and the like, as they are also of economical importance to humans. Thus, provided is the treatment of livestock, including, but not limited to, domesticated swine (pigs and hogs), ruminants, horses, poultry, and the like.

IV.B. Formulations

A therapeutic composition (e.g., a composition comprising an AT₂ receptor antagonist, a hormone or hormone conjugate, or a combination thereof) preferably comprises a composition that includes a pharmaceutically acceptable carrier. Suitable formulations include aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents.

The compositions used in the methods can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

The formulations can be presented in unit-dose or multi-dose containers, for example sealed ampoules and vials, and can be stored in a frozen or freeze-dried (lyophilized) condition requiring only the addition of sterile liquid carrier immediately prior to use.

For oral administration, the compositions can take the form of, for example, tablets or capsules prepared by a conventional technique with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycollate); or wetting agents (e.g., sodium lauryl sulphate). The tablets can be coated by methods known in the art. For example, an ACE inhibitor can be formulated in combination with hydrochlorothiazide, and as a pH stabilized core having an enteric or delayed release coating which protects the ACE inhibitor until it reaches the colon.

Liquid preparations for oral administration can take the form of, for example, solutions, syrups or suspensions, or they can be presented as a dry product for constitution with water or other suitable vehicle before use. Such liquid preparations can be prepared by conventional techniques with pharmaceutically acceptable additives such as suspending agents (e.g., sorbitol syrup, cellulose derivatives or hydrogenated edible fats); emulsifying agents (e.g. lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound. For buccal administration the compositions can take the form of tablets or lozenges formulated in conventional manner.

The compounds can also be formulated as a preparation for implantation or injection. Thus, for example, the compounds can be formulated with suitable polymeric or hydrophobic materials (e.g., as an emulsion in an acceptable oil) or ion exchange resins, or as sparingly soluble derivatives (e.g., as a sparingly soluble salt).

The compounds can also be formulated in rectal compositions (e.g., suppositories or retention enemas containing conventional suppository bases such as cocoa butter or other glycerides), creams or lotions, or transdermal patches.

IV.C. Doses

The term “effective amount” is used herein to refer to an amount of the therapeutic composition (e.g., a composition comprising an AT₂ receptor antagonist, a hormone or hormone conjugate, or a combination thereof) sufficient to produce a measurable biological response (e.g., a reduction in a biological activity of an AT₂ receptor). Actual dosage levels of active ingredients in a therapeutic composition of the invention can be varied so as to administer an amount of the active compound(s) that is effective to achieve the desired therapeutic response for a particular subject and/or application. The selected dosage level will depend upon a variety of factors including the activity of the therapeutic composition, formulation, the route of administration, combination with other drugs or treatments, severity of the condition being treated, and the physical condition and prior medical history of the subject being treated. Preferably, a minimal dose is administered, and dose is escalated in the absence of dose-limiting toxicity to a minimally effective amount. Determination and adjustment of a therapeutically effective dose, as well as evaluation of when and how to make such adjustments, are known to those of ordinary skill in the art of medicine. Table 2 can provide guidance in determining a suitable therapeutically effective dose.

For administration of a therapeutic composition as disclosed herein, conventional methods of extrapolating human dosage based on doses administered to a murine animal model can be carried out using the conversion factor for converting the mouse dosage to human dosage: Dose Human per kg=Dose Mouse per kg×12 (Freireich et al., (1966) Cancer Chemother Rep. 50:219-244). Drug doses can also be given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich et al. (Freireich et al., (1966) Cancer Chemother Rep. 50:219-244). Briefly, to express a mg/kg dose in any given species as the equivalent mg/sq m dose, multiply the dose by the appropriate km factor. In an adult human, 100 mg/kg is equivalent to 100 mg/kg×37 kg/sq m=3700 mg/m².

For oral administration, a satisfactory result can be obtained employing the AT₂ receptor antagonist in an amount ranging from about 0.01 mg/kg to about 100 mg/kg and preferably from about 0.1 mg/kg to about 30 mg/kg. A preferred oral dosage form, such as tablets or capsules, will contain the AT₂ receptor antagonist in an amount ranging from about 0.1 to about 500 mg, preferably from about 2 to about 50 mg, and more preferably from about 10 to about 25 mg.

For parenteral administration, the ACE inhibitor can be employed in an amount ranging from about 0.005 mg/kg to about 100 mg/kg, preferably about 10 to 50 or 10 to 70 mg/kg, and more preferably from about 10 mg/kg to about 30 mg/kg.

For additional guidance regarding formulation and dose, see U.S. Pat. Nos. 5,326,902; 5,234,933; PCT International Publication No. WO 93/25521; Berkow et al., (1997) The Merck Manual of Medical Information, Home ed. Merck Research Laboratories, Whitehouse Station, N.J.; Goodman et al., (1996) Goodman & Gilman's the Pharmacological Basis of Therapeutics, 9th ed. McGraw-Hill Health Professions Division, New York; Ebadi, (1998) CRC Desk Reference of Clinical Pharmacology. CRC Press, Boca Raton, Fla.; Katzung, (2001) Basic & Clinical Pharmacology, 8th ed. Lange Medical Books/McGraw-Hill Medical Pub. Division, New York; Remington et al., (1975) Remington's Pharmaceutical Sciences, 15th ed. Mack Pub. Co., Easton, Pa.; and Speight et al., (1997) Avery's Drug Treatment: A Guide to the Properties, Choice, Therapeutic Use and Economic Value of Drugs in Disease Management, 4th ed. Adis International, Auckland/Philadelphia; Duch et al., (1998) Toxicol. Lett. 100-101:255-263.

IV.D. Routes of Administration

Suitable methods for administering to a subject an AT₂ receptor antagonist or modulator, a hormone, hormone conjugate, or combination thereof in accordance with the methods of the present invention include but are not limited to systemic administration, parenteral administration (including intravascular, intramuscular, intraarterial administration), oral delivery, buccal delivery, subcutaneous administration, inhalation, intratracheal installation, surgical implantation, transdermal delivery, local injection, and hyper-velocity injection/bombardment. Where applicable, continuous infusion can enhance drug accumulation at a target site (see, e.g., U.S. Pat. No. 6,180,082).

The particular mode of drug administration used in accordance with the methods of the present invention depends on various factors, including but not limited to the vector and/or drug carrier employed, the severity of the condition to be treated, and mechanisms for metabolism or removal of the drug following administration.

V. Modulators of an Ang II Receptor

Ang II receptor modulators, a group that specifically includes AT₁ and AT₂ receptor antagonists, are employed in the present methods for modulating Ang II receptor activity in tissues, and in cell and tissue cultures. Thus, as used herein, the terms “modulate”, “modulating”, and “modulator” are meant to be construed to encompass inhibiting, blocking, promoting, stimulating, agonizing, antagonizing, or otherwise affecting Ang II receptor activity in cells and tissues, whether they be in vivo or in vitro.

AT₂ receptor modulators, a group that specifically includes AT₂ receptor antagonists, are employed in the present methods for modulating AT₂ receptor activity in tissues, and in cell and tissue cultures. Thus, as used herein, the terms “modulate”, “modulating”, and “modulator” are meant to be construed to encompass inhibiting, blocking, promoting, stimulating, agonizing, antagonizing, or otherwise affecting AT₂ receptor activity in cells and tissues, whether they be in vivo or in vitro.

Such modulators can take a variety of forms that include compounds that interact with AT₂ receptor in a manner such that functional interactions with natural AT₂ receptor ligands are mimicked, stimulated and/or inhibited. Exemplary modulators include analogs of an AT₂ receptor natural ligand, mimetics of a natural ligand of AT₂ receptor that mimic the structural region involved in an AT₂ receptor-ligand binding interactions, polypeptides having a structure or sequence corresponding to a natural ligand of an AT₂ receptor, and antibodies which immunoreact with either an AT₂ receptor or an AT₂ natural ligand, all of which exhibit modulator activity as defined herein.

V.A. Peptides and Polypeptides

In one embodiment, the invention provides Ang II receptor (preferably AT₂ receptor) modulators in the form of polypeptides. A polypeptide (a term which includes peptides and peptide mimetics) Ang II receptor (preferably AT₂ receptor) modulator can have the sequence characteristics of either a natural ligand of an Ang II receptor (preferably AT₂ receptor) or of an Ang II receptor (preferably AT₂ receptor) itself at the region involved in an Ang II receptor (preferably AT₂ receptor)-ligand interaction. A preferred Ang II receptor (preferably AT₂ receptor) modulator peptide corresponds in sequence to a natural ligand.

Many angiotensin II peptide mimetics and peptide analogs have been developed. Many of these exert agonistic and antagonistic effects. Some of these peptide mimetics and analogs are described in Clin. Sci. 57: 71, (1979); Endocrinol. 107(5): 1365, (1980); Clin. Sci. Mol. Med. 51: 4305, (1976); Am J. Physiol. 236(3): F252, (1976); Clin. Sci. Mol. Med. 51: 3855, (1976); J. Cardiovasc. Pharm. 5: 1025, (1983); Life Sci. 34: 317, (1983); Can J. Physiol Pharm. 57: 121, (1979); Can J. Physiol Pharm. 57: 763, (1979); J. Pharm. Pharmacol. 32: 232, (1980); J. Med. Chem. 20(2): 253, (1977); J. Med. Chem. 19(2): 244, (1976); J. Med. Chem. 13: 181, (1970); Circ. Res. 29: 664, (1971); Circ. Res. 46: 720, (1980); Proc. Nat'l Acad. Sci. USA 67:1624, (1970); J. Med. Chem. 20: 898, (1977); Cir. Res. 31: 862, (1972).

The term “polypeptide” refers to fusion proteins and polypeptides, recombinant proteins and polypeptides, peptide derivatives, amides, conjugates with proteins, cyclized peptides, polymerized peptides, analogs, mimetics, fragments, chemically modified peptides, and the like derivatives, as described below.

In one embodiment, an exemplary polypeptide comprises no more than about 100 amino acid residues, preferably no more than about 60 residues, more preferably no more than about 30 residues. Peptides can be linear or cyclic.

It is understood that a subject polypeptide need not be identical to the amino acid residue sequence or the chemical structure of a AT₂ receptor natural ligand. Preferably it includes required binding sequences and is able to function as AT₂ receptor modulator.

A subject polypeptide includes any analog, fragment or chemical derivative of a polypeptide that is an AT₂ receptor modulator, including mimetics. Such a polypeptide can be subject to various changes, substitutions, insertions, and deletions where such changes provide for certain advantages in its use. In this regard, a AT₂ receptor modulator polypeptide of this invention corresponds to, rather than is identical to, the sequence of a natural ligand where one or more changes are made and it retains the ability to function as a AT₂ receptor modulator in one or more of the assays as defined herein. Thus, a polypeptide can be in any of a variety of forms of peptide derivatives, that include amides, conjugates with proteins, cyclized peptides, polymerized peptides, analogs, fragments, chemically modified peptides, and the like derivatives.

The term “analog” includes any polypeptide having an amino acid residue sequence substantially identical to a sequence of a natural ligand of AT₂ receptor in which one or more residues have been conservatively substituted with a functionally similar residue and which displays AT₂ receptor modulator activity as described herein. Examples of conservative substitutions include the substitution of one non-polar (hydrophobic) residue such as isoleucine, valine, leucine or methionine for another; the substitution of one polar (hydrophilic) residue for another such as between arginine and lysine, between glutamine and asparagine, between glycine and serine; the substitution of one basic residue such as lysine, arginine or histidine for another; or the substitution of one acidic residue, such as aspartic acid or glutamic acid for another.

The phrase “conservative substitution” also includes the use of a chemically derivatized residue in place of a non-derivatized residue provided that such polypeptide displays the requisite inhibition activity.

The term “chemical derivative” refers to a subject polypeptide having one or more residues chemically derivatized by reaction of a functional side group. Such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups can be derivatized to form salts, methyl and ethyl esters or other types of esters or hydrazides. Free hydroxyl groups can be derivatized to form O-acyl or O-alkyl derivatives. The imidazole nitrogen of histidine can be derivatized to form N-im-benzylhistidine. Also included as chemical derivatives are those peptides that contain one or more naturally occurring amino acid derivatives of the twenty standard amino acids. For examples: 4-hydroxyproline can be substituted for proline; 5-hydroxylysine can be substituted for lysine; 3-methylhistidine can be substituted for histidine; homoserine can be substituted for serine; and ornithine can be substituted for lysine. Provided polypeptides also include any polypeptide having one or more additions and/or deletions or residues relative to the sequence of a natural ligand of an AT₂ receptor, so long as the requisite activity is maintained.

The term “fragment” refers to any subject polypeptide having an amino acid residue sequence shorter than that of a polypeptide sequence of a natural ligand of an AT₂ receptor.

When a provided polypeptide has a sequence that is not identical to the sequence of an AT₂ receptor natural ligand, it is typically because one or more conservative or non-conservative substitutions have been made, usually no more than about 30 number percent, and preferably no more than 10 number percent of the amino acid residues are substituted. Additional residues can also be added at either terminus of a polypeptide for the purpose of providing a “linker,” by which the provided polypeptides can be conveniently affixed to a label or solid matrix, or carrier. Labels, solid matrices and carriers that can be used with the polypeptides of this invention are described hereinbelow.

Amino acid residue linkers are usually at least one residue and can be 40 or more residues, more often 1 to 10 residues, but do not form AT₂ receptor ligand epitopes. Typical amino acid residues used for linking are tyrosine, cysteine, lysine, glutamic and aspartic acid, or the like. In addition, a subject polypeptide can differ, unless otherwise specified, from the natural sequence of an AT₂ receptor ligand by the sequence being modified by terminal-NH₂ acylation, e.g., acetylation, or thioglycolic acid amidation, by terminal-carboxylamidation, e.g., with ammonia, methylamine, and the like terminal modifications. Terminal modifications are useful, as is well known, to reduce susceptibility by proteinase digestion, and therefore serve to prolong half life of the polypeptides in solutions, particularly biological fluids where proteases can be present. In this regard, polypeptide cyclization is also a useful terminal modification, and is particularly preferred also because of the stable structures formed by cyclization and in view of the biological activities observed for such cyclic peptides as described herein.

Any peptide of the present invention can be used in the form of a pharmaceutically acceptable salt. Suitable acids which are capable of the peptides with the provided peptides include inorganic acids such as trifluoroacetic acid (TFA), hydrochloric acid (HCl), hydrobromic acid, perchloric acid, nitric acid, thiocyanic acid, sulfuric acid, phosphoric acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid or the like. HCl and TFA salts are particularly preferred.

Suitable bases capable of forming salts with the provided peptides include inorganic bases such as sodium hydroxide, ammonium hydroxide, potassium hydroxide and the like; and organic bases such as mono-di- and tri-alkyl and aryl amines (e.g. triethylamine, diisopropyl amine, methyl amine, dimethyl amine and the like), and optionally substituted ethanolamines (e.g. ethanolamine, diethanolamine and the like).

A provided peptide, also referred to herein as a subject polypeptide, can be synthesized by any of the techniques that are known to those skilled in the polypeptide art, including recombinant DNA techniques. Synthetic chemistry techniques, such as a solid-phase Merrifield-type synthesis, are preferred for reasons of purity, antigenic specificity, freedom from undesired side products, ease of production and the like. A summary of many available techniques can be found in Steward et al., Solid Phase Peptide Synthesis, W. H. Freeman Co., San Francisco, Calif. (1969); Bodanszky et al., Peptide Synthesis, John Wiley & Sons, Second Edition, (1976); Meienhofer, Hormonal Proteins and Peptides, 2:46, Academic Press, New York, N.Y., (1983); Merrifield, (1969) Adv. Enzymol. 32:221-96; Fields et al., (1990) Int. J. Peptide Protein Res. 35:161-214; and U.S. Pat. No. 4,244,946 for solid phase peptide synthesis, and Schroder et al., The Peptides, Vol. 1, Academic Press, New York, N.Y., (1965) for classical solution synthesis, each of which is incorporated herein by reference. Appropriate protective groups usable in such synthesis are described in the above texts and in McOmie, Protective Groups in Organic Chemistry, Plenum Press, New York, N.Y., (1973), which is incorporated herein by reference.

In general, the solid-phase synthesis methods provided comprise the sequential addition of one or more amino acid residues or suitably protected amino acid residues to a growing peptide chain. Normally, either the amino or carboxyl group of the first amino acid residue is protected by a suitable, selectively removable protecting group. a different, selectively removable protecting group is utilized for amino acids containing a reactive side group such as lysine.

Using a solid phase synthesis as exemplary, the protected or derivatized amino acid is attached to an inert solid support through its unprotected carboxyl or amino group. The protecting group of the amino or carboxyl group is then selectively removed and the next amino acid in the sequence having the complimentary (amino or carboxyl) group suitably protected is admixed and reacted under conditions suitable for forming the amide linkage with the residue already attached to the solid support. The protecting group of the amino or carboxyl group is then removed from this newly added amino acid residue, and the next amino acid (suitably protected) is then added, and so forth. After all the desired amino acids have been linked in the proper sequence, any remaining terminal and side group protecting groups (and solid support) are removed sequentially or concurrently, to afford the final linear polypeptide.

The resultant linear polypeptides prepared for example as described above can be reacted to form their corresponding cyclic peptides. An exemplary method for cyclizing peptides is described by Zimmer et al., Peptides pp. 393-394, ESCOM Science Publishers, B. V., (1993). Typically, tertbutoxycarbonyl protected peptide methyl ester is dissolved in methanol and sodium hydroxide solution are added and the admixture is reacted at 20° C. to hydrolytically remove the methyl ester protecting group. After evaporating the solvent, the tertbutoxycarbonyl protected peptide is extracted with ethyl acetate from acidified aqueous solvent. The tertbutoxycarbonyl protecting group is then removed under mildly acidic conditions in dioxane cosolvent. The unprotected linear peptide with free amino and carboxy termini so obtained is converted to its corresponding cyclic peptide by reacting a dilute solution of the linear peptide, in a mixture of dichloromethane and dimethylformamide, with dicyclohexylcarbodiimide in the presence of 1-hydroxybenzotriazole and N-methylmorpholine. The resultant cyclic peptide is then purified by chromatography.

V.B. Monoclonal Antibodies

The present invention describes, in one embodiment, Ang II receptor (preferably AT₂ receptor) modulators in the form of monoclonal antibodies which immunoreact with an Ang II receptor (preferably AT₂ receptor) and bind the Ang II receptor (preferably AT₂ receptor) to modulate Ang II receptor (preferably AT₂ receptor) biological activity as described herein. The invention also describes cell lines which produce the antibodies, methods for producing the cell lines, and methods for producing the monoclonal antibodies. Such antibodies are described, for example, in U.S. Pat. No. 6,063,620 to Vinson et al.

A monoclonal antibody of this invention comprises antibody molecules that 1) immunoreact with an isolated AT₂ receptor, and/or 2) bind to an AT₂ receptor to modulate its biological function.

The term “antibody or antibody molecule” in the various grammatical forms is used herein as a collective noun that refers to a population of immunoglobulin molecules and/or immunologically active portions of immunoglobulin molecules, i.e., molecules that contain an antibody combining site or paratope. An “antibody combining site” is that structural portion of an antibody molecule comprised of heavy and light chain variable and hypervariable regions that specifically binds antigen.

Exemplary antibodies for use in the present invention are intact immunoglobulin molecules, substantially intact immunoglobulin molecules, single chain immunoglobulins or antibodies, those portions of an immunoglobulin molecule that contain the paratope, including those portions known in the art as Fab, Fab′, F(ab′)2 and F(v), and also referred to as antibody fragments.

The phrase “monoclonal antibody” in its various grammatical forms refers to a population of antibody molecules that contain only one species of antibody combining site capable of immunoreacting with a particular epitope. A monoclonal antibody thus typically displays a single binding affinity for any epitope with which it immunoreacts. A monoclonal antibody can therefore contain an antibody molecule having a plurality of antibody combining sites, each immunospecific for a different epitope, e.g., a bispecific monoclonal antibody.

A monoclonal antibody is typically composed of antibodies produced by clones of a single cell called a hybridoma that secretes (produces) only one kind of antibody molecule. The hybridoma cell is formed by fusing an antibody-producing cell and a myeloma or other self-perpetuating cell line. The preparation of such antibodies was first described by Kohler & Milstein, (1975) Nature 256:495-497, which description is incorporated by reference. Additional methods are described by Zola, Monoclonal Antibodies: a Manual of Techniques, CRC Press, Inc, Boca Raton, Fla. (1987). The hybridoma supernates so prepared can be screened for the presence of antibody molecules that immunoreact with an AT₂ receptor and for inhibition of an AT₂ receptor biological function.

Briefly, to form the hybridoma from which the monoclonal antibody composition is produced, a myeloma or other self-perpetuating cell line is fused with lymphocytes obtained from the spleen of a mammal hyperimmunized with a source of an AT₂ receptor. It is preferred that the myeloma cell line used to prepare a hybridoma be from the same species as the lymphocytes. Typically, a mouse of the strain 129 GIX+ is a preferred mammal. Suitable mouse myelomas for use in the present invention include the hypoxanthine-aminopterin-thymidine-sensitive (HAT) cell lines P3X63-Ag8.653, and Sp2/0-Ag14 that are available from ATCC, Manassas, Va., under the designations CRL 1580 and CRL 1581, respectively.

Splenocytes are typically fused with myeloma cells using polyethylene glycol (PEG) 1500. Fused hybrids are selected by their sensitivity to HAT. Hybridomas producing a monoclonal antibody of this invention can be identified using an enzyme-linked immunosorbent assay (ELISA).

A provided monoclonal antibody can also be produced by initiating a monoclonal hybridoma culture comprising a nutrient medium containing a hybridoma that secretes antibody molecules of the appropriate specificity. The culture is maintained under conditions and for a time period sufficient for the hybridoma to secrete the antibody molecules into the medium. The antibody-containing medium is then collected. The antibody molecules can then be further isolated by employing techniques known to those of ordinary skill in the art. Media useful for the preparation of these compositions are both well known in the art and commercially available and include synthetic culture media, inbred mice and the like. An exemplary synthetic medium is Dulbecco's minimal essential medium (DMEM) Dulbecco et al., (1959) Virol. 8:396) supplemented with 4.5 gm/1 gm glucose, 20 mM glutamine, and 20% fetal calf serum. An exemplary inbred mouse strain is the Balb/C.

Other methods of producing a monoclonal antibody, a hybridoma cell, or a hybridoma cell culture are also well known. See, for example, the method of isolating monoclonal antibodies from an immunological repertoire as described by Sastry et al., (1989) Proc. Natl. Acad. Sci. U.S.A. 86:5728-5732; and Huse et al., (1989) Science 246:1275-1281.

It is also possible to determine, without undue experimentation, if a monoclonal antibody has the same (i.e., equivalent) specificity (immunoreaction characteristics) as a monoclonal antibody of this invention by ascertaining whether the former prevents the latter from binding to a preselected target molecule. If the monoclonal antibody being tested competes with the monoclonal antibody of the invention, as shown by a decrease in binding by the monoclonal antibody of the invention in standard competition assays for binding to the target molecule when present in the solid phase, then it is likely that the two monoclonal antibodies bind to the same, or a closely related, epitope.

Still another way to determine whether a monoclonal antibody has the specificity of a monoclonal antibody of the invention is to pre-incubate the monoclonal antibody of the invention with the target molecule with which it is normally reactive, and then add the monoclonal antibody being tested to determine if the monoclonal antibody being tested is inhibited in its ability to bind the target molecule. If the monoclonal antibody being tested is inhibited then, in all likelihood, it has the same, or functionally equivalent, epitopic specificity as the monoclonal antibody of the invention.

An additional way to determine whether a monoclonal antibody has the specificity of a monoclonal antibody of the invention is to determine the amino acid residue sequence of the CDR regions of the antibodies in question.

“CDRs” (complementarity determining regions) mean the three subregions of the light or heavy chain variable regions that have hypervariable sequences and form loop structures that are primarily responsible for making direct contact with antigen. Antibody molecules having identical, or functionally equivalent, amino acid residue sequences in their CDR regions have the same binding specificity. Methods for sequencing polypeptides are well known in the art.

The immunospecificity of an antibody, its target molecule binding capacity, and the attendant affinity the antibody exhibits for the epitope, are defined by the epitope with which the antibody immunoreacts. The epitope specificity is defined at least in part by the amino acid residue sequence of the variable region of the heavy chain of the immunoglobulin that comprises the antibody, and in part by the light chain variable region amino acid residue sequence. Use of the terms “having the binding specificity of” or “having the binding preference of” indicates that equivalent monoclonal antibodies exhibit the same or similar immunoreaction (binding) characteristics and compete for binding to a preselected target molecule.

Humanized monoclonal antibodies offer particular advantages over murine monoclonal antibodies, particularly insofar as they can be used therapeutically in humans. Specifically, human antibodies are not cleared from the circulation as rapidly as “foreign” antigens, and do not activate the immune system in the same manner as foreign antigens and foreign antibodies. Methods of preparing “humanized” antibodies are generally well known in the art, and can readily be applied to the antibodies of the present invention. Thus, the invention provides, in one embodiment, a monoclonal antibody of this invention that is humanized by grafting to introduce components of the human immune system without substantially interfering with the ability of the antibody to bind antigen.

The use of a molecular cloning approach to generate antibodies, particularly monoclonal antibodies, and more particularly single chain monoclonal antibodies, is also provided. The production of single chain antibodies has been described in the art. See, e.g., U.S. Pat. No. 5,260,203, the contents of which are herein incorporated by reference. For this, combinatorial immunoglobulin phagemid libraries are prepared from RNA isolated from the spleen of the immunized animal, and phagemids expressing appropriate antibodies are selected by panning on endothelial tissue. The advantages of this approach over conventional hybridoma techniques are that approximately 10⁴ times as many antibodies can be produced and screened in a single round, and that new specificities are generated by H and L chain combination in a single chain, which further increases the chance of finding appropriate antibodies. Thus, an antibody of the present invention, or a “derivative” of an antibody of the present invention pertains to a single polypeptide chain binding molecule which has binding specificity and affinity substantially similar to the binding specificity and affinity of the light and heavy chain aggregate variable region of an antibody described herein.

V.C. Other Modulators

Given the disclosure of the Ang II receptor (preferably AT₂ receptor) activity in tissues herein, it is also provided that other chemical compounds can be used to modulate Ang II receptor (preferably AT₂ receptor) activity in tissues in accordance with the methods of the present invention. The identification of such compounds is facilitated by the description of screening assays directed to Ang II receptor (preferably AT₂ receptor) activity in tissues presented below.

Particularly provided chemical entities do not naturally occur in any cell of a lower eukaryotic organism such as yeast. More particularly, provided chemical entities do not naturally occur in any cell, whether of a multicellular or a unicellular organism. Even more particularly, the provided chemical entity is not a naturally occurring molecule, e.g. it is a chemically synthesized entity.

In certain embodiments of the present invention, the modulator comprises PD123319, PD123317, or a combination of PD123319 and PD123317. PD123319 is commercially available from Sigma Chemical Company, St. Louis, Mo. PD123317 is available from Parke-Davis. In certain other embodiments of the present invention, the agent comprises any biologically active composition that leads to a decrease in expression, activity, or function of the AT₂ receptor.

V.D. Antisense Therapy

It is also provided according to the present invention that expression of an AT₂ receptor can be modulated in the vertebrate subject through the administration of an antisense oligonucleotide derived from a nucleic acid molecule encoding an AT₂ receptor, such as those described in U.S. Pat. No. 5,639,940, the entire contents of which are herein incorporated by reference, and those in the Examples presented below. Therapeutic methods utilizing antisense oligonucleotides have been described in the art, for example, in U.S. Pat. Nos. 5,627,158 and 5,734,033, the contents of each of which are herein incorporated by reference.

In one embodiment of present invention an antisense nucleic acid which is complementary to a sequence present in a modulatable, transcriptional sequence can be employed. The compound can also be a double-stranded nucleic acid or a nucleic acid capable of forming a triple helix with a double-stranded DNA.

Antisense compounds are commonly used as research reagents and diagnostics. For example, antisense oligonucleotides, which are able to inhibit gene expression with exquisite specificity, are often used by those of ordinary skill to elucidate the function of particular genes. Antisense compounds are also used, for example, to distinguish between functions of various members of a biological pathway. Antisense modulation has, therefore, been harnessed for research use.

The specificity and sensitivity of antisense is also harnessed by those of skill in the art for therapeutic uses. Antisense oligonucleotides have been employed as therapeutic moieties in the treatment of disease states in animals and man. Antisense oligonucleotides have been safely and effectively administered to humans and numerous clinical trials are presently underway. It is thus established that oligonucleotides can be useful therapeutic modalities that can be configured to be useful in treatment regimes for treatment of cells, tissues and animals, especially humans. In the context of this invention, the term “oligonucleotide” refers to an oligomer or polymer of RNA or DNA or mimetics thereof. This term includes oligonucleotides composed of naturally-occurring nucleobases, sugars and covalent internucleoside (backbone) linkages as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

While antisense oligonucleotides are a preferred form of antisense compound, the present invention comprehends other oligomeric antisense compounds, including but not limited to oligonucleotide mimetics such as are described below. The antisense compounds in accordance with this invention preferably comprise from about 8 to about 30 nucleobases (i.e. from about 8 to about 30 linked nucleosides). Particularly preferred antisense compounds are antisense oligonucleotides, even more preferably those comprising from about 12 to about 25 nucleobases. As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be inked to either the 2′, 3′ or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. Within the oligonucleotide structure, the phosphate groups are commonly referred to as forming the internucleoside backbone of the oligonucleotide. The normal linkage or backbone of RNA and DNA is a 3′ to 5′ phosphodiester linkage.

Specific examples of preferred antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

The antisense compounds used in accordance with this invention can be conveniently and routinely made through the well-known technique of solid phase synthesis. Equipment for such synthesis is commercially available, for example from Applied Biosystems (Foster City, Calif., United States of America). Any other means for such synthesis known in the art can additionally or alternatively be employed. It is well known to use similar techniques to prepare oligonucleotides such as the phosphorothioates and alkylated derivatives.

The antisense compounds of the invention can be synthesized in vitro and can optionally include or not include antisense compositions of biological origin, or genetic vector constructs designed to direct the in vivo synthesis of antisense molecules. The compounds of the invention can also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption.

The antisense compounds of the invention encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

The antisense compounds of the present invention can be administered to a subject by a variety of methods. Many of these methods and preparations for delivering an antisense compound of the present invention are disclosed in U.S. Pat. No. 6,372,492 to Bennett et al., incorporated herein by reference.

VI. Applications of the Present Invention

The ability to modulate AT₂ receptor biological activity as disclosed herein has a variety of applications. Some of the various applications of the present invention are described hereinbelow. Additional applications of the present invention will be apparent to those of ordinary skill in the art upon consideration of the present invention.

VI.A. Method of Modulating in vivo AT₂ Receptor Levels

A method for transcriptionally modulating in a multicellular organism the expression of a gene encoding Ang II receptor (preferably AT₂ receptor) as a treatment of a disorder associated with Ang II receptor (preferably AT₂ receptor) biological activity in a vertebrate subject is also provided in accordance with the present invention. In a preferred embodiment, this method comprises administering to the vertebrate subject a compound at a concentration effective to transcriptionally modulate expression of AT₂ receptor to thereby treat a cancer, preferably colorectal cancer, or to treat colorectal polyps. More preferably, the provided method reduces elevated levels of AT₂ receptor by inhibiting expression of AT₂ receptor to thereby treat a cancer, preferably colorectal cancer, or to treat colorectal polyps.

In this method the provided compound can optionally comprise an antibody or polypeptide prepared in accordance with the methods described above and which transcriptionally modulates expression of AT₂ receptor. Optionally, the antibody or polypeptide directly binds to DNA or RNA, or directly binds to a protein involved in transcription. Thus, indirect and direct transcriptional modulation is within the scope of the present method.

In an alternative embodiment of the present method the candidate compound does not naturally occur in the cell, specifically transcriptionally modulates expression of the gene encoding the protein of interest, and directly binds to DNA or RNA, or directly binds to a protein at a site on such protein which is not a ligand-binding domain of a receptor which naturally occurs in the cell. Preferably, the cell contacted in accordance with this method is a human cell.

Particularly provided chemical entities do not naturally occur in any cell of a lower eukaryotic organism such as yeast. More particularly, provided chemical entities do not naturally occur in any cell, whether of a multicellular or a unicellular organism. Even more particularly, the provided chemical entity is not a naturally occurring molecule, e.g. it is a chemically synthesized entity.

Optionally, the compound can bind to a modulatable transcription sequence of the gene. For example, the compound can bind to a promoter region upstream of a nucleic acid sequence encoding AT₂ receptor.

In the methods above, modulation of the transcription of an AT₂ receptor results in either upregulation or downregulation of expression of the gene encoding the protein of interest, depending on the identity of the molecule which contacts the cell. Preferably, the provided method reduces elevated levels of an AT₂ receptor by inhibiting expression of an AT₂ receptor to thereby treat a cancer, preferably colorectal cancer, or to treat colorectal polyps.

VI.B. Method of Decreasing a Biological Function of an AT₂ Receptor

In another aspect of the present invention, a method of decreasing a biological function of an AT₂ receptor in a subject in need thereof is disclosed. In a preferred embodiment, the method comprises administering an effective amount of a therapeutic agent to the subject to decrease a biological function of an AT₂ receptor.

When a rat AT₂ receptor is employed, the AT₂ receptor preferably comprises an amino acid sequence encoded by a cDNA as disclosed by Kambayashi et al., (1993) J. Biol. Chem. 268:24543-24546, and/or by Mukoyama et al., (1993) J. Biol. Chem. 268:24539-24542. When a human AT2 receptor is employed, the AT2 receptor preferably comprises an amino acid sequence encoded by a cDNA as disclosed by Chassagne et al., (1995) Genomics 25 (2), 601-603. When a mouse receptor is employed, the AT₂ receptor preferably comprises an amino acid sequence encoded by a cDNA as disclosed by Ichiki et al., (1994) Biochim. Biophys. Acta 1189 (2), 247-250, by Nakajima et al., (1993) Biochem. Biophys. Res. Commun. 197 (2): 393-399 or by Nahmias et al., (1996) Adv. Exp. Med. Biol. 396,167-173.

Several of the various biological functions of an AT₂ receptor have been discussed herein above. A preferred biological function of an AT₂ receptor includes ligand binding ability, for example the ability to bind angiotensin II (or a mimetic or analog thereof), as well as the compounds PD 123317 and PD 123319. Other biological functions of an AT₂ receptor flow from the ligand binding event. That is, a biological function that can be decreased includes any cascade effects that are initiated by the binding of a ligand by an AT₂ receptor. These biological functions can be known functions or unknown effects, or can be suspected functions or effects.

A subject can be any vertebrate or invertebrate organism. Alternatively, a subject can comprise one or more cells maintained in vivo or in vitro (e.g. a cell culture). Further, a subject can also comprise a tissue that is maintained in vivo or in vitro (e.g. a tissue culture). Preferred subjects include mice and humans.

As noted hereinabove, the precise quantity of “an effective amount” can vary from subject to subject and from therapeutic agent to therapeutic agent. Thus, an effective amount generally comprises an amount of therapeutic agent that is sufficient to achieve a desired result, such as a decrease in a biological function of an AT₂ receptor.

A range of therapeutic agents and potential therapeutic agents are described in the present disclosure. Preferred therapeutic agents include the compounds PD 123317 and PD 123319 and antisense molecules. However, it is not necessary that a therapeutic agent be identified as such. Thus, suspected and candidate therapeutic agents fall within the scope of that term. Additionally, a therapeutic agent can also comprise buffers, excipients, cofactors (which can include peptides, nucleic acids and small molecules), and other compounds as well. Considerations for therapeutic agent formulations are discussed hereinabove.

The therapeutic agent can be administered by employing any of a variety of techniques. Some example methods of administering a therapeutic agent are presented hereinabove. For example, a therapeutic agent can be administered orally, by injection or topically, to name just a few methods.

VI.C. Method of Preventing the Development of a Cancer in a Subject

In one aspect of the present invention, a method of preventing the development of a cancer in a subject having an AT₂ receptor. In a preferred embodiment, the method comprises: (a) providing a subject having an AT₂ receptor; and (b) administering to the subject a therapeutically effective amount of an AT₂ receptor antagonist, whereby the development of a cancer in a subject having an AT₂ receptor is prevented.

When a rat AT₂ receptor is employed, the AT₂ receptor preferably comprises an amino acid sequence encoded by a cDNA as disclosed by Kambayashi et al., (1993) J. Biol. Chem. 268:24543-24546, and/or by Mukoyama et al., (1993) J. Biol. Chem. 268:24539-24542. When a human AT2 receptor is employed, the AT2 receptor preferably comprises an amino acid sequence encoded by a cDNA as disclosed by Chassagne et al., (1995) Genomics 25 (2), 601-603. When a mouse receptor is employed, the AT₂ receptor preferably comprises an amino acid sequence encoded by a cDNA as disclosed by Ichiki et al., (1994) Biochim. Biophys. Acta 1189 (2), 247-250, by Nakajima et al., (1993) Biochem. Biophys. Res. Commun. 197 (2): 393-399 or by Nahmias et al., (1996) Adv. Exp. Med. Biol. 396,167-173.

A subject can be any vertebrate or invertebrate organism. Alternatively, a subject can comprise one or more cells maintained in vivo or in vitro (e.g. a cell culture). Further, a subject can also comprise a tissue that is maintained in vivo or in vitro (e.g. a tissue culture). Preferred subjects include mice and humans.

The present invention can be employed to prevent or inhibit the development a cancer. Cancers that are particularly suitable to prevention or inhibition of development include colorectal cancers, however the present invention is not limited to these types of cancers. An illustrative, but non-limiting list of cancers that can be prevented or inhibited from developing include fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstroom's macroglobulinemia, and heavy chain disease.

As noted hereinabove, the precise quantity of “an effective amount” can vary from subject to subject and from therapeutic agent to therapeutic agent. Thus, an effective amount generally comprises an amount of therapeutic agent that is sufficient to achieve a desired result, such as a decrease in a biological function of an AT₂ receptor.

A range of therapeutic agents and potential therapeutic agents are described in the present disclosure. Preferred therapeutic agents include the compounds PD 123317 and PD 123319 and antisense molecules. However, it is not necessary that a therapeutic agent be identified as such. Thus, suspected and candidate therapeutic agents fall within the scope of that term. Additionally, a therapeutic agent can also comprise buffers, excipients, cofactors (which can include peptides, nucleic acids and small molecules), and other compounds as well. Considerations for therapeutic agent formulations are discussed hereinabove.

The therapeutic agent can be administered by employing any of a variety of techniques. Some example methods of administering a therapeutic agent are presented hereinabove. For example, a therapeutic agent can be administered orally, by injection or topically, to name just a few methods.

VI.D. Method of Treating a Cancer

In yet another aspect of the present invention, a method of treating a cancer in a subject having an AT₂ receptor is disclosed. In a preferred embodiment, the method comprises: (a) providing a subject having a cancer and an AT₂ receptor; (b) administering to the subject a therapeutically effective amount of an AT₂ receptor antagonist, whereby a cancer in a subject having an AT₂ receptor is treated.

When a rat AT₂ receptor is employed, the AT₂ receptor preferably comprises an amino acid sequence encoded by a cDNA as disclosed by Kambayashi et al., (1993) J. Biol. Chem. 268:24543-24546, and/or by Mukoyama et al., (1993) J. Biol. Chem. 268:24539-24542. When a human AT₂ receptor is employed, the AT₂ receptor preferably comprises an amino acid sequence encoded by a cDNA as disclosed by Chassagne et al., (1995) Genomics 25 (2), 601-603. When a mouse receptor is employed, the AT₂ receptor preferably comprises an amino acid sequence encoded by a cDNA as disclosed by Ichiki et al., (1994) Biochim. Biophys. Acta 1189 (2), 247-250, by Nakajima et al., (1993) Biochem. Biophys. Res. Commun. 197 (2): 393-399 or by Nahmias et al., (1996) Adv. Exp. Med. Biol. 396, 167-173.

A subject can be any vertebrate or invertebrate organism. Alternatively, a subject can comprise one or more cells maintained in vivo or in vitro (e.g. a cell culture). Further, a subject can also comprise a tissue that is maintained in vivo or in vitro (e.g. a tissue culture). Preferred subjects include mice and humans.

The present invention can be employed to treat a cancer. Cancers that are particularly suitable to treatment include colorectal cancers, however the present invention is not limited to these types of cancers. An illustrative, but non-limiting list of cancers that can be prevented or inhibited from developing include fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenströom's macroglobulinemia, and heavy chain disease.

As noted hereinabove, the precise quantity of “a therapeutically effective amount” can vary from subject to subject and from therapeutic agent to therapeutic agent. Thus, a therapeutically effective amount generally comprises an amount of therapeutic agent that is sufficient to achieve a desired result, such as a decrease in a biological function of an AT₂ receptor.

A range of therapeutic agents and potential therapeutic agents are described in the present disclosure. Preferred therapeutic agents include the compounds PD 123317 and PD 123319 and antisense molecules. However, it is not necessary that a therapeutic agent be identified as such. Thus, suspected and candidate therapeutic agents fall within the scope of that term. Additionally, a therapeutic agent can also comprise buffers, excipients, cofactors (which can include peptides, nucleic acids and small molecules), and other compounds as well. Considerations for therapeutic agent formulations are discussed hereinabove.

The therapeutic agent can be administered by employing any of a variety of techniques. Some example methods of administering a therapeutic agent are presented hereinabove. For example, a therapeutic agent can be administered orally, by injection or topically, to name just a few methods.

VI.E. Method of Screening a Candidate Compound

A candidate substance identified according to a screening assay of the present invention has an ability to modulate the biological activity of an AT₂ receptor polypeptide. In a preferred embodiment, such a candidate compound can have utility in the treatment of disorders and conditions associated with the biological activity of an AT₂ receptor polypeptide, including, but not limited to prevention and/or treatment of a cancer, particularly a colorectal cancer.

In a cell-free system, the method comprises the steps of establishing a control system comprising an AT₂ receptor polypeptide and a ligand which is capable of binding to the polypeptide; establishing a test system comprising an AT₂ receptor polypeptide, the ligand, and a candidate compound; and determining whether the candidate compound modulates the activity of the polypeptide by comparison of the test and control systems. Representative ligands can comprise PD 123317, PD 123319, saralasin, losartan, a peptide or a small molecule, and in this embodiment, the biological activity or property screened can include binding affinity.

In another embodiment of the invention, a form of an AT₂ receptor polypeptide or a catalytic or immunogenic fragment or oligopeptide thereof, can be used for screening libraries of compounds in any of a variety of drug screening techniques. The fragment employed in such a screening can be affixed to a solid support. The formation of binding complexes, between an AT₂ receptor polypeptide and the agent being tested, will be detected. In a preferred embodiment, an AT₂ receptor polypeptide has an amino acid sequence comprising SEQ ID NOs: 2 or 4.

Another technique for drug screening which can be used provides for high throughput screening of compounds having suitable binding affinity to the protein of interest as described in published PCT application WO 84/03564, herein incorporated by reference. In this method, as applied to a polypeptide of the present invention, large numbers of different small test compounds are synthesized on a solid substrate, such as plastic pins or some other surface. The test compounds are reacted with the polypeptide, or fragments thereof. Bound polypeptide is then detected by methods well known to those of skill in the art. The polypeptide can also be placed directly onto plates for use in the aforementioned drug screening techniques.

In yet another embodiment, a method of screening for a modulator of an AT₂ receptor polypeptide comprises: providing a library of test samples; contacting an AT₂ receptor polypeptide with each test sample; detecting an interaction between a test sample and an AT₂ receptor polypeptide; identifying a test sample that interacts with an AT₂ receptor polypeptide; and isolating a test sample that interacts with an AT₂ receptor polypeptide.

In each of the foregoing embodiments, an interaction can be detected spectrophotometrically, radiologically or immunologically. An interaction between an AT₂ receptor polypeptide and a test sample can also be quantified using methodology known to those of skill in the art.

In accordance with the present invention there is also provided a rapid and high throughput screening method that relies on the methods described above. This screening method comprises separately contacting each of a plurality of substantially identical samples with an AT₂ receptor polypeptide and detecting a resulting binding complex. In such a screening method the plurality of samples preferably comprises more than about 10⁴ samples, and more preferably comprises more than about 5×10⁴ samples.

VI.F. Method of Identifying Compounds Which Inhibit Ligand Binding

In one aspect of the present invention, an assay method for identifying a compound that inhibits binding of a ligand to an AT₂ receptor polypeptide is disclosed. A known ligand of an AT₂ receptor can be used in the assay method as the ligand against which the inhibition by a test compound is gauged. PD 123317, PD 123319, saralasin, and combinations thereof, are preferred ligands in the assay method. The method comprises (a) incubating an AT₂ receptor polypeptide with a ligand in the presence of a test inhibitor compound; (b) determining an amount of ligand that is bound to the AT₂ receptor polypeptide, wherein decreased binding of ligand to the AT₂ receptor polypeptide in the presence of the test inhibitor compound relative to binding in the absence of the test inhibitor compound is indicative of inhibition; and (c) identifying the test compound as an inhibitor of ligand binding if decreased ligand binding is observed.

In another aspect of the present invention, the disclosed assay method can be employed in the structural refinement of candidate an AT₂ receptor antagonists. For example, multiple rounds of optimization can be followed by gradual structural changes in a strategy of inhibitor design.

In this application the determining can be performed by employing any suitable method. For example, radiological, spectrophotometric and immunological methods can be employed. Additional methods will be apparent to those of ordinary skill in the art upon consideration of the present disclosure.

VI.G. Administration of an ACE Inhibitor and an AT₂ Receptor Antagonist

In a further embodiment of the present invention, an ACE inhibitor and an AT₂ antagonist can be employed to achieve one of treating a cancer and preventing a cancer. In a preferred embodiment, the method comprises: (a) providing subject in need of treatment; (b) administering an ACE inhibitor to the subject; and (c) administering an AT₂ receptor antagonist to a subject.

A subject can be any vertebrate or invertebrate organism. Alternatively, a subject can comprise one or more cells maintained in vivo or in vitro (e.g. a cell culture). Further, a subject can also comprise a tissue that is maintained in vivo or in vitro (e.g. a tissue culture). Preferred subjects include mice and humans.

The present invention can be employed to treat a cancer. Cancers that are particularly suitable to treatment include colorectal cancers, however the present invention is not limited to these types of cancers. An illustrative, but non-limiting list of cancers that can be prevented or treated includes fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenströom's macroglobulinemia, and heavy chain disease.

Any compound known or suspected to be an ACE inhibitor can be employed in the present invention. An illustrative but non-limiting list of ACE inhibitors includes Captopril, Enalapril, Lisinopril, Benazepril, Quinapril, Ramipril, Trandolapril, Moexipril, Fosinopril, Perindep and pharmaceutically acceptable salts thereof.

Similarly, any compounds known or suspected to by an AT2 receptor antagonist can be employed in the present invention. An illustrative but non-limiting list of AT2 receptor antagonists includes AT2 receptor antagonist is selected from the group consisting of candesartan cilexetil, eprosartan, irbesartan, tasosartan, telmisartan, valsartan, BMS-184699, 3-(2′-(tetrazol-5-yl)-1,1′-biphen-4-yl)methyl-5,7-dimethyl-2-ethyl-3H-imidazo[4,5-b]pyridine, BAY 106734, BIBR363, CL329167, E4177, EMD73495, HN65021, HR720, HOE720, LRB081, SC52459, SL910102, UP2696, YM358, EMD66397, ME3221, TAK536, BMS 184698, CGP42112A, CGP49870, CP14R130, E4188, EMD666R4, EXP9954, FRI 153332, GA0050, KT3579, LF70156, LRB057, LY266099, LY301875, PD 123177, PD 126055, SC51757, SC54629, U96849, UK77778, WAY126227, WK1260, WK1492, YH1498, and YM31472, PD 123317, PD 123319, Saralasin, and Losartan.

VII. Conculsions

The present invention discloses the observation that inhibition of a cancer can be achieved by modulation the biological activity of an AT₂ receptor. Biological activity can include expression of the receptor. This observation can be advantageously employed in a variety of application, including prevention and/or inhibition of a cancer, treatment of a cancer and screening of AT₂ receptor modulatory compounds, to name just a few applications. The present invention is particularly applicable to prevention, inhibition and treatment of colorectal cancers.

### EXAMPLES

The following Examples have been included to illustrate preferred modes of the invention. Certain aspects of the following Examples are described in terms of techniques and procedures found or provided by the present inventors to work well in the practice of the invention. These Examples are exemplified through the use of standard laboratory practices of the inventors. In light of the present disclosure and the general level of skill in the art, those of skill will appreciate that the following Examples are intended to be exemplary only and that numerous changes, modifications and alterations can be employed without departing from the spirit and scope of the invention.

### Materials and Methods Employed in Examples 2-6

Materials.

Ang II was purchased from Peninsula Laboratories (Belmont, Calif., United States of America). Azoxymethane (AOM) and the AT₂ receptor blocker PD123,319 were from Sigma Chemical Co. (St. Louis, Mo., United States of America). The protease inhibitor cocktail Complete was from Boehringer Mannheim (Mannheim, Germany). Anti-human cytochrome P4502E1 polyclonal antibodies were raised in Dr. F. P. Guengerich's laboratory (Vanderbilt University, Nashville, Tenn., United States of America) and were presented as a gift. The anti-O⁶-methyldeoxyguanosine monoclonal antibody was a generous gift from Dr. M. F. Rajewsky (Institute of Cell Biology, University of Essen Medical School, Germany). The enhanced chemiluminescence (ECL) Western blotting detection system was from Amersham Pharmacia Biotech, Inc. (Piscataway, N.J., United States of America). Primers for PCR were synthesized by the DNA Synthesis & Reagent Supply Core facility in the Vanderbilt University Diabetes Center. All other chemicals were of analytical grade.

Animals and Genotyping.

The original male hemizygote AT₂-null mutant (Agtr2^{-/y}) mice were the offspring of Agtr2 deletion mutants produced by homologous recombination in embryonic stem cells derived from strain 129/OIa (Ichiki et al., (1995), Nature 377:748-750). Chimeric males were mated with C57BL/6J females such that the genetic background of the mutants consisted of 129/OIa and C57BL/6J. Wild type male littermates served as controls. In order to introduce a tumor-susceptible genetic background, heterozygote female mice (Agtr2^{-/x}) were mated with SWR/J male mice. F₂ male hemizygote AT₂-null mice were compared with wild type male littermates (Agtr2^{x/y}) in the study. Southern blot analysis of tail DNA was used to screen for the Agtr2 genotype as described by Ichiki et al., (1995) Nature 377:748-750. All animals were maintained in a humidity- and temperature-controlled room on 12 h light/dark cycles. All procedures for handling animals were approved by the Institutional Committee for Animal Care and Use of Vanderbilt University.

Experimental Protocol for Azoxymethane Administration in vivo.

All AOM-handling procedures were approved by the office of Safety and Environmental Health of Vanderbilt University. Ten-week-old male wild type and AT₂-null mice (minimum 5 mice/group) received regular mouse chow (#5015, Purina Mills, Inc., Indianapolis, Ind., United States of America). Mice were treated with four consecutive weekly administrations of AOM (10 mg/kg, I.P.) for tumorigenic study or with a bolus intraperitoneal administration of AOM (10 mg/kg) for short term study. AT₂ receptor blocker PD123319 treatment (15 mg/kg/12 h, gavage administration and 50 μg/ml in drinking tap water) was initiated 3 h prior to the AOM treatment. The control group for the AOM treatment received saline. Mice for the short term study were sacrificed 6 h or 24 h after AOM treatment. Mice for the tumorigenic study were sacrificed 23-26 weeks after the first AOM treatment. The colons and livers were macroscopically examined. For quantitative estimation of tumor burdens, the colons were removed, longitudinally opened and placed flat on filter paper. Tumor size was measured with a caliper. The measurements were carried out blindly by a single observer throughout the study. Randomly selected tumors and adjacent normal tissues were fixed with 10% formalin, sectioned, and stained with H&E for histological examination. The remaining tissues were frozen in liquid nitrogen and stored at −80° C.

Preparation of Microsomal Fractions.

Tissues were individually homogenized by a Polytron homogenizer in three volumes of 1 mM Tris-HCl buffer, pH 7.5, containing 1 mM EDTA, 0.25 M sucrose and protease inhibitor cocktail. The supernatant from 10,000× g centrifugation of the homogenate for 15 min was further centrifuged at 100,000× g for 1 h. The microsomal membranes were suspended in 100 mM NaH₂PO₄, pH 7.4, 10 mM MgCl₂, 20% glycerol, and protease inhibitor cocktail, and 25 μg of the membrane protein was subjected to Western blot analysis.

Western Blot Analysis.

The liver microsomal membranes were lysed with 0.5 ml lysis buffer (10 mM Tris-HCl (pH 7.4), 1% SDS). After sonication, the lysate was boiled for 5 min. The boiled lysate was subjected to 10% SDS-polyacrylamide gel electrophoresis. Proteins were then transferred to a nitrocellulose membrane by electroblotting. The membrane was blocked for 16 hours at 4° C. with non-fat milk in Tris-buffered saline (TBS, 10 mM Tris (pH 7.5), 100 mM NaCl) containing 0.1% Tween-20, and then incubated with anti-rabbit anti-human CYP2E1 antibodies for 1 hour. After washing, the membrane was incubated for 1 hour with goat anti-rabbit IgG conjugated with horseradish peroxidase; peroxidase activity was visualized with an ECL Western blotting detection system.

DNA Isolation.

Immediately after the mice were sacrificed by cervical dislocation, the colon was excised, washed with phosphate-buffered saline, divided into proximal and distal segments, and snap-frozen in liquid N₂. Small portions of each lobe of the liver were also removed and frozen in the same manner. Tissue was digested in a lysis buffer consisting of 10 mM Tris-HCl, pH 7.4, 1 mM EDTA, 1% SDS, 5× SSC and 0.2 mg/ml Proteinase K. DNA was extracted by phenol/chloroform/isoamyl alcohol and precipitated with ice-cold ethanol by standard protocols. Samples were then treated with RNase A (50 mg/ml) and DNA was fragmented by sonication. The concentration of DNA was determined by the absorbance at 260 nm.

Immuno-Slot-Blot Assay.

The immuno-slot-blot method described by Thomale et al. (Thomale et al., (1996) in Pfeifer, G. P. (ed.), Technologies for Detection of DNA Damage and Mutations, Plenum Press, New York, N.Y.) was used with the following modifications. DNA samples were heat-denatured for 10 min, immediately chilled on ice, and mixed with an equal volume of 2 M ammonium acetate. The resulting single-stranded DNA was then immobilized on a nitrocellulose membrane and fixed to the membrane by UV crosslinking. The membrane was then treated with TBS containing 5% skim milk for 2 h. The membrane was first incubated overnight at 4 C with a monoclonal antibody raised against O⁶-methyldeoxyguanosine. After washing, the membrane was incubated for 1 h with goat anti-rabbit IgG conjugated with horseradish peroxidase (Amersham Life Sciences). DNA-methyl adducts were visualized using the ECL Western blotting detection system. Relative blot intensities were measured by densitometry using a Fluor-S image analyzer (Bio-Rad Laboratories, Inc., Hercules, Calif., United States of America).

Isolation of Total RNA and Reverse Transcription-Polymerase Chain Reaction (RT-PCR) of AT₂ Receptor mRNA.

Liver and colonic tissues were ground in liquid nitrogen, and total RNA was extracted by TRI reagent (Sigma) according to the manufacturer's protocol. RT-PCR was carried out using the same conditions and primers for the AT₂ receptor as described by Tamura et al., (2000) J. Hypertens. 18:1239-1246.

Statistical Analysis.

Data obtained from the Western blot analysis and immuno-slot-blot assay were averaged and are presented as means ±S.E. Significant differences between groups were evaluated by one-way analysis of variance with the Student-Newman-Keuls test. A value of P<0.05 was considered significant.

### Example 1

PD123319, an angiotensin II receptor (AT2 receptor) antagonist, is available in 1-mg, 10-mg, 50-mg, 100-mg, 250 mg, 500 mg, and 1000 mg tablets for oral administration and in 1-mg, 10-mg, 50-mg, 100-mg, 500 mg, 1000 mg, 5000 mg, and 10,000 mg suppositories for rectal administration.

PD123319 is indicated for the treatment of colorectal cancer including non-malignant polyps of the colon and colorectal adenocarcinoma. There are no known contradictions to treatment with PD123319.

A 4-8 week course of treatment, by oral administration, results in a significant reduction of colon polyp or adenocarcinoma number as determined by endoscopy. A 12-20 week course of therapy yields a remission of adenocarcinoma (e.g., tumor number and/or volume) in greater than 79% of individuals that are diagnosed with at least one adenocarcinoma. Total dose per day is determined by severity (number and/or cancer stage) as assessed by a treating veterinarian or physician. A preferred course of treatment for a patient with colorectal polyps is 10-30 mg (medication) per kg (body weight) per day. A preferred course of treatment for a patient with colorectal adenocarcinoma is 20-30 mg/kg/day. In certain cases, a course of treatment for a patient with colorectal adenocarcinoma is 100 mg/kg/day administered orally or as a suppository. A preferred maintenance or chemoinhibitory course of therapy is typically 1.0 to 10 mg/kg. A preferred maintenance or chemoinhibitory course of therapy for individuals with genetic predisposition to colorectal cancer, including individuals with FAP or HPNNC, is 10-30 mg/kg/day. Of course with respect to the above presented ranges and to ranges presented elsewhere herein, amounts of 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29 an and 30 mg/kg are included.

Certain dosages of PD123319 by body weight are provided in TABLE 2, below.

TABLE 2
PD123319 DOSING BY BODY WEIGHT

| Weight | Total Dose Per Day (mg/day) | | | | |
|---|---|---|---|---|---|
| Kg | 0.1 mg/kg | 0.6 mg/kg | 1 mg/kg | 10 mg/kg | 100 mg/kg |
| 40 | 4 | 20 | 40 | 400 | 4000 |
| 50 | 5 | 25 | 50 | 500 | 5000 |
| 70 | 7 | 35 | 70 | 700 | 7000 |
| 100 | 10 | 50 | 100 | 1000 | 10000 |
| 120 | 12 | 60 | 120 | 1200 | 12000 |
| 150 | 15 | 75 | 150 | 1500 | 15000 |

### Example 2

### AOM-Induced Colon Adenocarcinoma in AT₂ Receptor-Knockout Mice

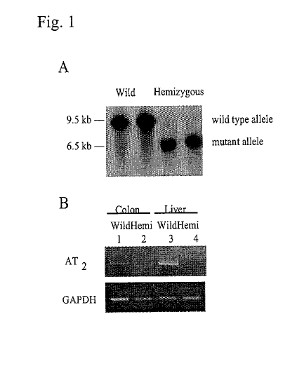

This Example pertains to the examination of the role of the Ang II-AT₂ receptor in azoxymethane (AOM)-induced colon adenocarcinoma by utilizing AT₂ receptor-deficient (AT₂-KO) mice. The genotype and expression of the mRNA were confirmed by Southern blot analysis and RT-PCR, respectively. Genomic DNA of male wild type and hemizygous mice exhibited single bands of 9.5 Kb or 6.5 Kb, respectively, in agarose gel electrophoresis (FIG. 1A). Results from RT-PCR indicated that AT₂-null mutant mice did not express AT₂ receptor mRNA in any tissue examined (FIG. 1B). Thus, the targeted disruption of the AT₂ receptor gene was effective and specific.

Four consecutive weekly IP administrations of AOM (10 mg/kg) caused the development of colon tumors in all wild type control mice at 23 weeks after the first AOM injection, but only one tumor was present in AT₂-KO mice (Table 3). In addition, the size of the tumors in wild type mice was large (over 55% of tumors 22 mm³). The tumors were predominantly observed in the lower half of the descending colon. Histological analysis revealed that the majority of the tumors were adenoma and a few were adenocarcinoma presumably due to early analysis. These results indicate that the AT₂ receptor-mediated signal is essential in AOM-induced colon adenocarcinoma in this mouse strain (a crossbreed of mouse strains 129/OIa and C57BL/6J). The results can also suggest that an alteration in the bioactivation and/or detoxification of AOM is involved in the resistance to tumorigenesis in AT₂-KO mice since the tumor multiplicity and size differences between the AT₂-KO mice and wild type mice are very clear.

TABLE 3
Summary of tumor results in mouse strain crossbred from 129/Ola
and C57BL/6J. Mice in both control and AT₂-KO groups,
10 weeks old, were treated with AOM (10 mg/kg,
4 consecutive weeks, I.P.). Mice were sacrificed
at 23 weeks after the initial injection of AOM.

| | | | | Colon Tumors | | |
|---|---|---|---|---|---|---|
| | Treat- | | Body | Total | Prevalence | Multi- |
| Group | ment | Number | Weight (g) | Number | (%) | plicity |
| Control | Saline | 7 | 36.9 ± 4.2 | 2 | 14.3 | 0.28 |
| | AOM | 8 | 36.5 ± 4.3 | 59 | 100 | 7.38 |
| AT₂-KO | Saline | 6 | 38.7 ± 3.0 | 1 | 16.7 | 0.17 |
| | AOM | 9 | 36.5 ± 1.6 | 1 | 11.1 | 0.11 |

Prevalence (%) = percent of tumor-bearing mice.
Multiplicity = average number of tumors/mouse.

### Example 3

### Preparation of Congenic AT₂-KO Mouse Strain with SWR/J Genetic Background

In Example 2, clear-cut results were obtained that the AT₂ receptor is involved in AOM-induced colon cancer. A concern was that this might be true only in the mouse strain crossbred from 129/OIa and C57BL/6J mouse strains. In order to overcome this problem, the AT₂-KO mice in this crossbred strain were back-crossed with SWR/J mice, whose susceptibility to AOM is established. During the course of back-crossing, similar experiments were performed as described in Example 1, using F₂ male wild and AT₂-KO mice. Similar results were again obtained, indicating that the disruption of the AT₂ receptor markedly attenuates AOM-induced tumorigenesis in the colon (Table 4).

Although AT₂-KO mice in this F₂ strain developed tumors, presumably due to the strong tumor susceptibility characteristic of the SWR/J background, the tumor multiplicity and volume in the KO mice were significantly smaller than in the wild type mice. This additional experiment assured the authenticity of the observations in Example 1. The preparation of congenic AT₂-KO mice with an SWR/J background has been accomplished to the 6^{th} generation.

TABLE 4
Summary of tumor results in mouse strain crossbred from above
strain (Table 1) and SWR/J.
F₂ male (wild and AT₂-KO) mice from female
AT₂-KO mice (AT₂^{-/+},
crossbreed of 129/Ola and C57BL/6J) and male SWR/J
mice were treated with AOM (10 mg/kg,
4 consecutive weeks, I.P.) Mice were
sacrificed at 26 weeks after the initial injection of AOM.

| | | | Colon Tumors | | | |
|---|---|---|---|---|---|---|
| | Treat- | Num- | Total | Prevalence | Multi- | Volume |
| Group | ment | ber | Number | (%) | plicity | (mm³) |
| Control | Saline | 10 | 3 | 10.0 | 0.3 | 7.2^{a} |
| | AOM | 8 | 96 | 100.0 | 12.0 | 36.8 ± 3.2* |
| AT₂-KO | Saline | 9 | 2 | 11.1 | 0.2 | 5.5^{a} |
| | AOM | 8 | 50 | 100.0 | 6.3 | 19.8 ± 3.0 |

Prevalence (%) = percent of tumor-bearing mice.
Multiplicity = average number of tumors/mouse.
Volume = total tumor volume (mm³)/number of tumors.
^{a}only one mouse spontaneously developed tumors.
*P < 0.05, compared with AT₂-KO mice.

### Example 4

### Preparation of Polyclonal Anti-AT₂ Receptor Antibodies

Since the AT₂ receptor is known to be expressed in a wide variety of tissues in the fetus, immunotolerance is probably established under physiological conditions. Therefore, in order to obtain a probe to label the AT₂ receptor, polyclonal antibodies were raised to the N-terminus fragmental peptide (MKDNFSFAATSRNIT) (SEQ ID NO: 8), intracellular third loop peptide (31CLP, GIRKHLLKTNSYGKNRITR DQVLK) (SEQ ID NO: 9), and N-terminus end peptide of the extramembrane section of the C-terminus (QQKLRSVFRVPITWL) (SEQ ID NO: 10) by utilizing AT₂-KO mice. The titers of all three antibodies are high enough to study immunohistochemical staining of the AT₂ receptor. In addition, the anti-AT₂-3ICLP antibodies have been found to be useful for Western blot analysis.

### Example 5

### AT2 Receptor Expression in Mice

Radioligand-Receptor Binding Study:

Receptor-binding with [¹²⁵I] CGP42112A (CGP) was originally used for the determination of the AT₂ receptor expression levels in the liver and colon. However, it was observed that the determination of the AT₂ receptor expression level by [¹²⁵I] CGP-binding is troublesome because of 1) relatively poor reproducibility and 2) the presence of [¹²⁵I] CGP-binding protein in the monocyte plasma membranes, in which the colon is rich (Egidy et al., (1997) Am J Physiol. 273:C826-33). Accordingly, overall AT₁ and AT₂ receptor expression in the liver and colon mucosa was reevaluated by utilizing receptor binding with [¹²⁵I]Sar¹·Ile⁸·Ang II. The expression levels of the Ang II type 1 (AT₁) and AT₂ receptors in the liver plasma membranes were 37.4±7.2 and 8.7±2.0 fmol/mg protein, respectively, but colon plasma membrane levels of both receptors were relatively low (6.9±1.1 and 3.3±2.1 fmol/mg protein) in 10-12 week old normal wild type mice (n=6). In the AT₂-KO mice, AT₁ receptor levels in the liver and colon plasma membranes were 62.0±10.7 and 7.5±2.0 fmol/mg protein (n=6), respectively. The AT₂ receptor was undetectable.

Western Blot Analysis:

Levels of the two Ang II receptors in the liver were analyzed by Western blot with anti-AT₁ receptor antibodies (Dr. Rakugi, Osaka University, Japan) and anti-AT₂ receptor antibodies. Both AT₁ and AT₂ receptors were detected similarly in the plasma membrane and nuclei. However, the nuclear AT₁ receptor showed a larger molecular size (46.7 kd) than the plasma membrane receptor (43.1 kd), implicating different posttranslational modification.

Thus, both AT₁ and AT₂ receptors were detected in the wild type mouse liver plasma membrane. However, only the AT₁ receptor was detected in the colon plasma membrane. These results indicate that the AT₂ receptor is expressed in normal mouse liver but little in colon mucosa. A noteworthy observation in this experiment is that the AT₁ receptor expression is significantly higher in the AT₂-KO mouse liver than in the wild type liver.

Cytochrome P450 Expression in Mouse Liver:

Anti-rabbit anti-human CYP1A1/1A2 and CYP2E1 antisera were obtained from Dr. F. P. Guengerich, Department of Biochemistry, Vanderbilt University. See Soucek et al., (1995) Biochem. 34:16013-16021; Shimada et al., (1992) Mol. Pharmacol. 41:856-864. The cross-reactivities of these antibodies to mouse CYP1A1/1A2 and 2E1 have been characterized. Both immunohistochemical study and Western blot analysis have revealed that these antibodies recognize mouse CYP proteins in normal untreated mouse liver and colon epithelium.

The inducibilities of CYP1A1, 1A2 and 2E1 in the liver were exaimed. AOM treatment (15 mg/kg, bolus I.P. injection) significantly increased the levels of cytochrome P450 proteins in wild type mouse liver but not in AT₂-KO mice liver. A noteworthy observation is that hepatic cytochrome P450 levels decreased substantially 24 h after AOM injection only in AT₂-KO mice. This differential inducibility of cytochrome P450s suggests that the AT₂ receptor-mediated signal is involved in hepatic cytochrome P450 induction and resultant procarcinogen metabolism. In addition, although AOM is reported to be metabolized mainly by CYP2E1 (Sohn et al., (1991) Carcinogenesis 12:127-131), these results can suggest that CYP1A1 and/or CYP1A2 could also be involved in AOM-induced tumorigenesis in the colon.

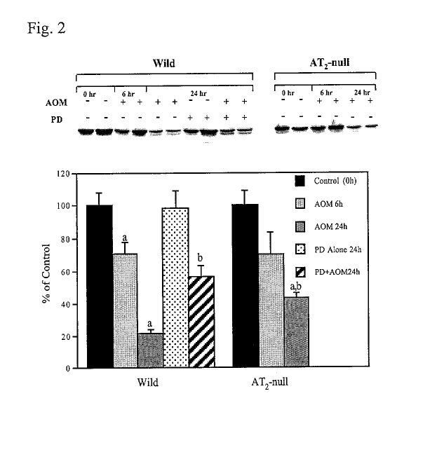

Thus, the effect of AT₂ receptor expression on CYP2E1 expression in the liver was examined. Although basal expression levels of the hepatic CYP2E1 proteins in the wild type and the AT₂-null mice were identical, their responses to AOM injection were significantly different (FIG. 2). AOM (10 mg/kg, bolus I.P. injection) significantly downregulated CYP2E1 protein expression in both wild type and AT₂-null mice livers. However, the expression levels were higher in the AT₂-null mouse liver than in the wild type mouse liver at 24 h after AOM injection. This observation coincided with results obtained following pharmacological attenuation of the AT₂ receptor function with the subtype-specific receptor antagonist PD123,319. Pretreatment of wild type animals with PD123319 (15 mg/kg/12 h by gavage administration and 0.35 mg/kg/h in drinking water) significantly attenuated AOM-induced CYP2E1 downregulation in the liver (FIG. 2). The extent of the attenuation was similar to that observed in the AT₂-null mice. Agreement between these two methods of attenuating the AT₂ receptor function indicates that the differing responses of CYP2E1 expression to AOM in the two mouse strains are due to a disruption of the AT₂ receptor function. These results indicate that wild type mice lose hepatic CYP2E1 enzyme activity more quickly and significantly than AT₂-null mice after AOM administration.

### Example 6

### A₂ Receptor Deficiency Decreased AOM-Induced DNA Adduct Formation

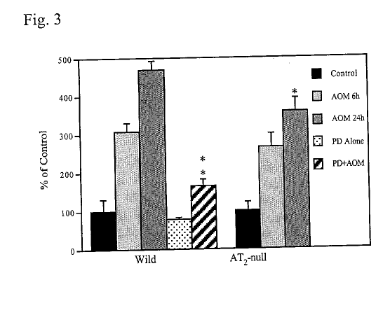

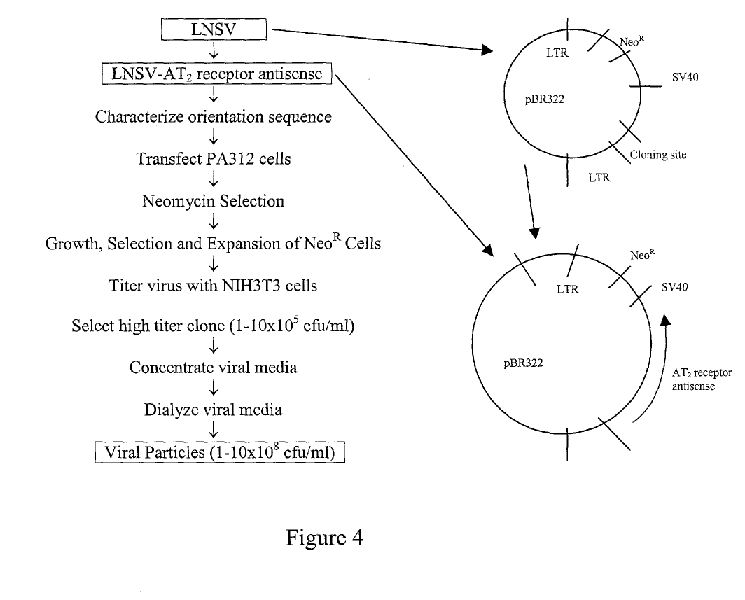

DNA adduct formation is the earliest step in chemical carcinogen-induced tumorigenesis. AOM increases O⁶-methylguanine adduct levels in the liver and colon, and this increase in the colon epithelium is apparently associated with AOM-induced colon tumorigenesis (Pegd, (1984) Cancer Invest. 2:223-231; Hamilton et al., (1988) Cancer Res, 48, 3313-8). To evaluate if disruption of the Agtr2 gene attenuates DNA methyl adduct formation in the colon epithelium, the levels of O⁶-methylguanine adduct in the colon were determined by the immuno-slot-blot method with an anti-O⁶-methyldeoxyguanosine antibody (Thomale et al., (1996) in Pfeifer, G. P. (ed.), Technologies for Detection of DNA Damage and Mutations, Plenum Press, New York, N.Y.). Bolus intraperitoneal administration of AOM (10 mg/kg) time-dependently increased colonic O⁶-methylguanine levels in both wild type and AT₂-null mice (FIG. 3). However, the adduct level in the wild type mice was significantly higher than that in the AT₂-null mice 24 h after the AOM administration. Pharmacological attenuation of the AT₂ receptor function in wild type mice with PD123319 (15 mg/kg/12h by gavage administration and 0.35 mg/kg/h in drinking water) markedly attenuated the AOM-induced increase in the colonic O⁶-methylguanine level 24 h after the AOM administration, although this attenuation was significantly larger than that observed in the AT₂-null mice. These results strongly suggest that the disruption of the AT₂ receptor gene attenuates DNA methyl adduct formation by AOM.

### Example 7

### Preparation of the Adenoviral AT₂ Receptor Transfection Vector

Preparation of Replication-Defective Adenovirus:

A replication-defective adenovirus containing the mouse AT₂ receptor (AdAT₂R) has been prepared by using the cosmid and terminal protein complex method, which is an efficient method for constructing recombinant adenoviruses utilizing cosmid cassettes and adenovirus DNA-terminal protein. Miyake, S., et al., Proc Natl Acad Sci USA. 1996; 93:1320-4. This adenovirus has deletions in the E1A, E1B and E3 genes and contains the cytomegalovirus (CMV) promoter that drives mouse AT₂ receptor cDNA expression following a growth hormone polyadenylation signal.

A₂ Receptor Expression in Cultured Intestinal Epithelial Cells:

The rat intestinal epithelial (RIE) cells were obtained from Dr. Raymond DuBois' laboratory, Gastroenterology Division, Department of Medicine, Vanderbilt University. RIE cells express the AT₁ receptor abundantly, but AT₂ receptor expression is negligible. To express the AT₂ receptor in RIE cells, cells were infected with AdAT₂R for 24 hrs, and then the expression levels of the AT₂ receptor were analyzed by radioligand-receptor binding assay. Quantitative analyses of AT₂ receptor expression showed 71, 92 and 131-fold enhancement of RIE cells infected at 25, 50 and 100 adenovirus multiplicity of infection (MOI), respectively.

### Example 8

### Effect of Ang II Receptor Blockers and an ACE Inhibitor on AOM-Induced Aberrant Crypt Foci Formation

Male SWR/J mice and crossbred mice (C57BL/6J and 129/OIa), which were primarily used in Examples 1-3 above, are employed. Both SWR/J and the crossbred wild type mice are sensitive to AOM-induced colon cancer, and the crossbred mice exhibited a sensitive reduction in AOM-induced ACF formation by an AT₂ receptor blocker. Each strain of mice, 10 weeks old, is divided into seven groups (minimum 7 mice/group). Mice are treated throughout the experiment with the AT₂ receptor antagonist PD123319 (1, 5 or 30 mg/kg/day), the AT₁ receptor-specific antagonist losartan (1 or 10 mg/kg/day), or the ACE inhibitor captopril (30 mg/kg/day). These six groups of mice plus one control group are injected with AOM (10 mg/kg, 4 consecutive weeks, I.P.). The Ang II receptor blockers and ACE inhibitor are dissolved in the drinking tap water, and the treatment is started one week prior to the AOM injection. The doses of the blockers and the inhibitor are accurately determined by measuring daily water consumption.

Mice in all seven groups are sacrificed at 10 weeks after the initial AOM injection. After fixation of the colon with 10% buffered formalin and staining of the mucosal surface with 0.2% methylene blue, the total number of ACF and the number of crypts per focus are quantified. If needed, pathological characterization of the ACF is also performed after hematoxylin eosin staining. Pathological sample preparation, sectioning and staining is done in the Human Tissue Acquisition Shared Resource in the Vanderbilt-Ingram Cancer Center, Vanderbilt University, Nashville, Tenn., United States of America.

Mouse groups treated with the AT₂ receptor blocker PD123319 or the ACE inhibitor captopril should have a smaller number of large ACF (multiple crypts per focus), which tend to develop tumors. If only the AT₂ receptor-mediated signal is associated with susceptibility to AOM-induced tumorigenesis, the AT₁ receptor blocker losartan likely has no effect on the induction of large ACF in mouse colon. However, since AT₁ receptor blockade has been shown to increase the plasma Ang II level (Tanaka, M., et al., Bioch Biophys Res Commu. 1999; 258:194-198), an increased level of ligand for the AT₂ receptor could increase the multiplicity of ACF formation per mouse. If the AT₁ receptor-mediated signal is also involved in AOM metabolism, the AT₁ receptor antagonist should also attenuate ACF formation.

ACE inhibition lowers the endogenous level of the ligand for the Ang II receptors, upregulates AT₁ receptor density, and increases bradykinin and/or substance P levels. The consequences of increased levels of bradykinin and/or substance P and upregulation of the AT₁ receptor in AOM-induced colon tumorigenesis are evaluated, as is the effect of an ACE inhibitor on AOM-induced tumorigenesis.

### Example 9

### Effect of Ang II Subtype-Specific Receptor Blockers and an ACE Inhibitor on AOM-Induced Tumorigenesis in Mice

Male SWR/J mice and crossbred wild type mice (C57BL/6J and 129/OIa) are utilized, since both SWR/J and crossbred strains are sensitive to AOM-induced colon cancer and SWR/J mice are well-characterized in relation to AOM-induced colon tumorigenesis. Each strain of mouse, 10 weeks old, is divided into seven groups (minimum 10 mice/group). Mice are treated throughout the experiment with the AT₂ receptor-specific antagonist PD123319 (30 mg/kg/day), the AT₁ receptor-specific antagonist losartan (10 mg/kg/day), or the ACE inhibitor captopril (30 mg/kg/day) with or without AOM (10 mg/kg, I. P., weekly for 4 weeks). The Ang II receptor blockers and ACE inhibitor are dissolved in the drinking tap water, and the treatments are started one week prior to the AOM injection. The listed doses of the blockers and the inhibitor are the depressor doses for hypertensive animals and are accurately determined by measuring daily water consumption. However, the doses of the drugs are adjusted by the results obtained from Laboraory Example 8. One group is given only tap water and 4 weekly I.P. administrations of AOM. The three drug control groups receive 4 weekly I.P. administrations of saline.

Mice in all seven groups are sacrificed at 26 weeks after the initial AOM or saline injection. Immediately after sacrifice the total number of tumors and their sizes are quantified. If needed, pathological characterization of tumors is also performed after 10% buffered formalin fixation and hematoxylin eosin staining. Pathological sample preparation, sectioning and staining is done in the Human Tissue Acquisition Shared Resource in the Vanderbilt-Ingram Cancer Center, Vanderbilt University, Nashville, Tenn., United States of America.

Mouse groups treated with the AT₂ receptor blocker PD123319 or the ACE inhibitor captopril should have a lower tumor incidence and tumor multiplicity. If only the AT₂ receptor-mediated signal is associated with susceptibility to AOM-induced tumorigenesis, the AT₁ receptor blocker losartan likely has no effect on the incidence and multiplicity of the tumors in mice. However, since AT₁ receptor blockade has been shown to increase the plasma Ang II level, an increased level of ligand for the AT₂ receptor could increase tumor incidence in mice. If AT₁ receptor function is also involved in AOM-induced tumorigenesis, the AT₁ receptor blocker should attenuate tumor incidence in the colon.

The Ang II receptor blockers or the ACE inhibitor could show more of a clearcut inhibitory effect on tumorigenesis in the crossbred mice (C57BL/6J and 129/OIa) than in the SWR/J mice. Comparison of the effects of the Ang II receptor blockade in the two strains likely indicates the extent of the involvement of Ang II receptor function in AOM susceptibility.

Doses of the Ang II receptor blockers are resolved by the in vivo study described in Example 8. ACE inhibition lowers the endogenous level of the ligand for the Ang II receptors, upregulates AT₁ receptor density, and increases bradykinin and/or substance P levels. The consequences of increased levels of bradykinin and/or substance P and upregulation of the AT₁ receptor in AOM-induced colon tumorigenesis is evaluated, as is the first to evaluate the effect of an ACE inhibitor on AOM-induced tumorigenesis.

### Example 10

### Effect Of AT₂ Receptor Blockade on the Growth of Human Colon Cancer Cells

Human colorectal cancer cell lines are selected based on Ang II receptor status and growth speed. Ang II receptor status is evaluated by [¹²⁵I]Sar¹·Ile⁸, AngII-binding in the presence or absence of AT₁ or AT₂ receptor-specific antagonists. If a cell line is encountered with AT₁ and/or AT₂ receptor expression and with an appropriate growth speed, this cell line is utilized for inoculation. A preliminary screen of one cell line, HT-29, revealed moderate AT₁ and AT₂ receptor expression.

Male athymic nude mice, 8 weeks old, are maintained on standard mouse chow. Mice are divided into five groups (7 mice/group). Four groups of mice are subcutaneously inoculated on both flanks with one million viable human colon cancer cells selected as described above. One control group of mice receives a vehicle solution of phosphate-buffered saline. Drug treatment is started two days before the inoculation and continued throughout the experiment. One group of mice each is treated with either the AT₂ receptor blocker PD123319 (30 mg/kg/day), the AT₁ receptor blocker losartan (10 mg/kg/day) or the ACE inhibitor captopril (30 mg/kg/day) through the drinking tap water. One inoculated group and the control group of mice are not treated. Although depressor doses of the receptor blockers and ACE inhibitor for hypertensive animals are chosen for this study, the results from the Examples presented above are also taken into consideration. Accordingly, the doses of the blockers and inhibitor can be adjusted.

Xenograft tumor size are measured weekly with calipers, and the tumor burden is expressed as tumor size, which is calculated by the following formula: volume=width²×length/2. Four to five weeks after inoculation, if the volume of the xenograft reaches 500 mm³, mice are sacrificed. Although no drug toxicity is expected, if mice exhibit signs of treatment-related discomfort, they are sacrificed immediately by an approved method.

The AT₂ receptor blocker PD123319 or the ACE inhibitor captopril should attenuate the growth of xenografts. If only the AT₂ receptor blocker, but not the ACE inhibitor or the AT₁ receptor blocker, inhibits growth of the xenografts, it is interpreted that the AT₂ receptor-mediated signal alone is involved in tumor growth. However, if the ACE inhibitor exhibits a stronger attenuation of the xenograft growth than the AT₂ receptor blocker or if only the ACE inhibitor attenuates the growth of xenografts, the side effects of the ACE inhibitor, such as an increase in bradykinin and/or substance P levels or a scavenging of free radicals, are also taken into consideration. If the AT1 receptor blocker inhibits the growth of xenografts, it is suggested that an Ang II-AT₁ receptor-mediated cell growth mechanism is involved in tumor growth. If no effect on the growth of xenografts is seen, it is concluded that components of the renin angiotensin system are not likely to be involved in tumor growth.

### Example 11

### Effect of AT₂ Receptor Disruption on the Growth of Lewis Lung Carcinoma Cells

Lewis lung carcinoma cells (LLC) are derived from the C57BL mouse strain and are tumorigenic in C57BL mice. Wild type C57BL/6J mice and AT₂-KO mice with a C57BL/6J genetic background are utilized. Both wild and KO male mice, 8 weeks old, 10 mice/group, are maintained on standard mouse chow. Both groups of mice are subcutaneously inoculated on both flanks with two million viable LLC cells. Xenograft tumor size is measured weekly as described in Example 10. Estimation of tumor burden and care of the mice is given as mentioned above. If the tumor growth in wild type mice is faster than in the AT₂-KO mice, another group of wild type mice is treated with the AT₂ receptor-specific blocker PD123319 (30 mg/kg) from two days prior to the LLC cell inoculation to the end of the experiment. Tumor size is measured as mentioned above. The AT₂ receptor expression in the tumor tissues and surrounding stromal tissues is examined by immunohistochemical staining with anti-AT₂ antibodies.

If the growth of xenografts in the AT₂-KO mice is slower than in wild type mice and if tumor growth in the wild type mice is attenuated by the AT₂ receptor-specific blocker PD123319, it is interpreted that the host AT₂ receptor function is positively involved in tumor growth. In either case, AT₂ receptor expression in the tumor tissue and/or surrounding stromal tissue is examined by immunohistochemical analysis. If the growth of the xenografts in the two groups is identical, it is interpreted that the host AT₂ receptor does not play a critical role in tumor growth.

Measurement of the expression level of the AT₂ receptor in the surrounding stromal tissues can be challenging because of low expression levels. In this case the stromal tissue is dissected around the tumor, the RNA extracted, and the AT₂ receptor mRNA level quantified by quantitative RT-PCR. RT-PCR will be performed by the standard procedure described by Tamura et al., (2000) J. Hypertens. 18:1239-1246.

### Example 12

### Prevention of AOM-Induced Colon Adenocarcinoma by AT₂ Receptor Antisense

This Example examines the effect of genetic control of AT₂ receptor expression, by expressing its antisense mRNA, on AOM-induced colon adenocarcinoma. A retroviral delivery system is employed. Although the retroviral DNA delivery system has advantages and disadvantages, its long term (possibly permanent) modification of the target gene expression is appropriate for practical cancer prevention. Furthermore, since AT₂-KO mice do not show any phenotypical health problems under normal conditions (Ichiki, T., et al., Nature 1995; 377:748-750; Hein, L., et al., Nature 1995; 377:744-7) the AT₂ receptor gene is a very good target gene for genetic modulation.

Preparation of an AT₂ Receptor Antisense cDNA Construct in a Retroviral Vector:

Procedures for the preparation of AT₂ receptor cDNA containing Hind III sites at both 5′ and 3′ ends, recombination of the AT₂ receptor cDNA with an LNSV retroviral vector and preparation of high titer viral medium essentially follow methods developed for AT₁ receptor antisense by Wang et al., (2000) Method. Enzymol. 314:581-590, with modification.

Preparation of AT₂ Receptor cDNA:

A pair of AT₂ receptor-specific primers with added Hind III sites (sense, 5′-CCMGCTTGCTTCAGCC TGCATTTTAAGGAGTG-3′ (SEQ ID NO: 11); antisense, 5′-CCMGCTTGTGCATCCCACTAACAGATTTAAGA-3′ (SEQ ID NO: 12) are used to generate AT₂ receptor cDNA by reverse transcriptase-polymerase chain reaction (RT-PCR). The total RNA (5 μg) from rat pheochromocytoma cell line PC12W cells is subjected to the RT reaction using SuperScript II (GIBCO-BRL, Gaithersburg, Md., United States of America) by standard protocols. Ausubel, F. M., et al., Protocols in Molecular Biology, New York: Greene Publishing Associates and John Wiley & Sons, 1992; Lu, D., Yu, K. and Raizada, M. K., Proc Natl Acad Sci USA. 1995; 92:1162-1166. Twenty pmol of AT₂ receptor sense and antisense primers are used and PCR will be done by standard protocols. Tamura, M., et al., J Hypertens 2000; 18:1239-1246; Lu, D., Yu, K. and Raizada, M. K., Proc Natl Acad Sci USA. 1995; 92:1162-1166. PCR products are separated on a 1% agarose gel. The appropriate 1.23 kb band is isolated and purified using a QIAEX™ II gel extraction kit (Qiagen, Chatsworth, Calif., United States of America). The identity of the 1.23-kb AT₂ receptor cDNA is confirmed by restriction enzyme analysis and sequencing. The identity of AT₂ receptor cDNA corresponding to nucleotides (nt) −120 to +1241 of the coding region of the AT₂ receptor is established and confirmed.

Recombination of AT₂ Receptor cDNA with the LNSV Retroviral Vector:

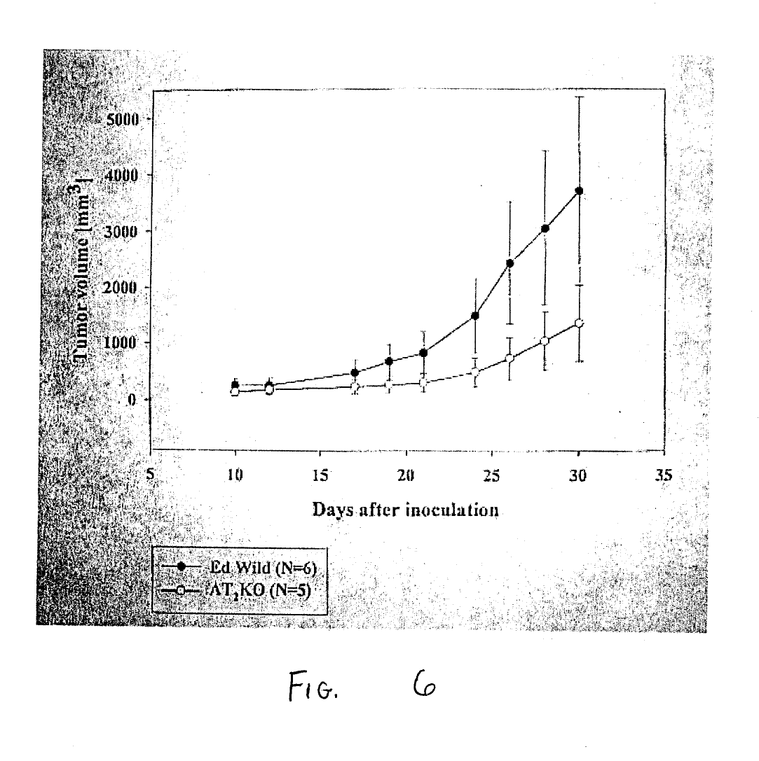

Standard protocols are utilized for all of the molecular biological techniques used in the preparation of an LNSV-AT₂ receptor antisense vector. Ausubel, F. M., et al., Protocols in Molecular Biology, New York: Greene Publishing Associates and John Wiley & Sons, 1992; Sambrook, T., Fritsch, E. F. and Maniatis, T., Molecular Cloning: A Laboratory Manual (2nd ed.). Plainview, N.Y.: Cold Spring Harbor Laboratory Press, 1989. AT₂ receptor cDNA (100 ng) and LNSV (retroviral vector, 50 ng) are digested with HindIII. The fragments are purified, mixed with ligase buffer (50 mM Tris-HCl, pH 7.5, 7 mM MgCl₂, 1 mM ATP) and 2 units of T₄ ligase (Stratagene, La Jolla, Calif., United States of America) and incubated overnight at 12 C. Recombinant DNA are transformed into competent HB101 bacterial cells, and AT₂ receptor antisense colonies are selected. The colonies that produce the 1.23 kb AT₂ receptor antisense are then grown in LB medium with ampicillin (100 μg/ml), and recombinant DNA is purified with a plasmid purification kit (Promega, Madison, Wis.) according to the protocol provided by the company. The protocol for the preparation of viral particles containing AT₂ receptor antisense is outlined in FIG. 6.

Evaluation of Retroviral Delivery and Expression of the AT₂ Receptor Antisense cDNA in vitro:

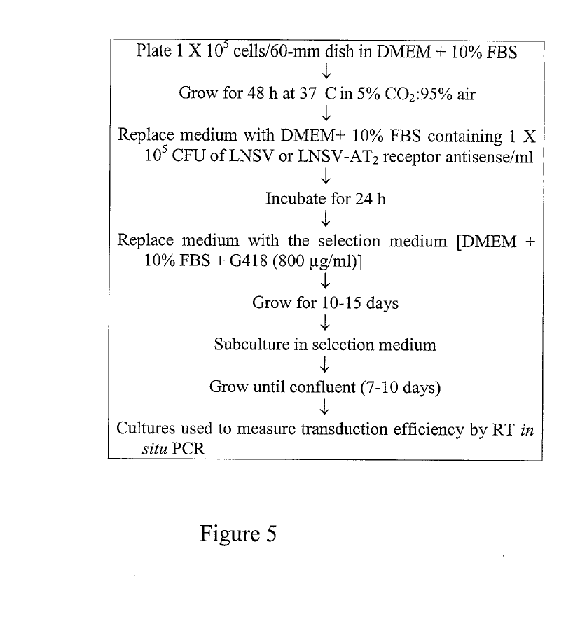

Adrenal medulla, brain and liver express the AT₂ receptor and are potential Ang II target tissues. Thus, rat pheochromocytoma cell line PC12 cells and human hepatocellular carcinoma-derived cell line HepG2 cells in culture are used to demonstrate the efficiency of transduction of the AT₂ receptor antisense in vitro. The protocol is outlined in FIG. 5.

AT₂ receptor antisense transcript localization in the cells is determined by RT in situ PCR. The experimental procedure for RT in situ PCR is performed as described elsewhere. Ausubel, F. M., et al., Protocols in Molecular Biology, New York: Greene Publishing Associates and John Wiley & Sons, 1992; Lu D., Yu, K. and Raizada, M. K., Proc Natl Acad Sci USA. 1995; 92:1162-1166. The expression is confirmed by Northern blot analysis. Tamura, M., et al., Hypertension 1999; 33:626-632. These experiments establish a protocol for infection of Ang II target cells with the retroviral vector and demonstrate stable expression of the AT₂ receptor antisense.

Effect of the AT₂ Receptor Antisense Expression on AOM-Induced Colon Tumorigenesis:

Three-week-old SWR/J mice are used to determine the efficiency of AT₂ receptor antisense transduction and the consequences of its expression in AOM-induced colon tumorigenesis. Mice are anesthetized, and viral particles containing 1×10⁹ CFU of either LNSV (control) or LNSV-AT2 receptor antisense are injected intraperitoneally into each mouse. Mice are allowed to recover. Expression of AT₂ receptor antisense in various Ang II target tissues is examined by RT-PCR 10 days after viral injection. Both LNSV and LNSV-AT₂ receptor antisense-injected animals are allowed to grow. Control (vector) and antisense groups are each then be divided into two groups (total 4 groups, minimum 10 mice/group) at the age of 8-10 weeks old. AOM or saline (control) treatment (10 mg/kg, 4 consecutive weeks, I.P.) is carried out as described herein above. Tumor development in both groups is evaluated as described in the Examples presented above at 23-26 weeks after the initial AOM injection.

It is anticipated that the mice transfected with AT₂ receptor antisense will develop significantly less numbers of and smaller size tumors in the colon as compared with the control mice which receive vector alone. Once the above expected results are obtained the following are evaluated: i) timing of the AT₂ receptor antisense-containing retroviral injection (weanling vs adult), ii) the most appropriate viral titer, and iii) administration routes. Simultaneously, the possibility of false-positive results is eliminated by a negative control experiment with LNSV-AT₂ receptor sense or mismatch cDNA.

Although it is proposed to transfect the AT₂ receptor antisense into weanling mice through I.P. injection, an alternative technique to effectively infect the virus into the liver is to inject the viral solution into the liver through the portal vein. If the crossbred wild type mouse strain (C57BL/6J and 129/OIa) exhibits AT₂ receptor blocker-dependent attenuation of AOM-induced colon cancer more so than SWR/J mice per the Examples presented above, crossbred mouse strain for this 1^{st} trial instead of SWR/J mice are employed.

### Example 13

### Effect of Hepatic AT₂ Receptor Expression on Chemical Carcinogen-Induced Cell Transformation in Colon Epithelial Cells

This Example evaluates the strength of the hepatic S 9 fraction (supernatant from 9000× g centrifugation of liver homogenate; source for xenobiotic bioactivation enzymes) in carcinogen-dependent transformation of colon epithelial cells in vitro. This in vitro study is a good approach to define the effect of AT₂ receptor disruption on chemical carcinogen-dependent tumorigenesis, since bioactivation enzymes from different animals and conditions can be examined in the identical system.

A mouse colonic epithelial cell line derived from the Immorto-Min mouse hybrid, carrying the AP^{min} mutation (APC^{+/-}), named the IMCE (Immorto-Min colonic epithelial) cell line, has been established. Whitehead, R. H. and Joseph, J. L., Epithelial Cell Biol. 1994; 3:119-25. These cells are transformed easily by ras activation (D'Abaco, G. M., Whitehead, R. H. and Burgess, A. W., Mol Cell Biol. 1996; 16:884-91) or β-catenin inactivation. The established cells are incubated with liver S 9 fractions in cofactor solution containing NADP and glucose-6-phosphate. Liver S 9 fractions are prepared from control wild type or AT₂-KO mouse liver following treatment with or without AOM (10 mg/kg, I.P.) and sacrifice. Timing of the liver dissection and preparation of the S 9 fraction are tested at 3 h and 6 h after AOM treatment. After incubation with the S 9 fraction for 24 h, cells are cultured at 33 C for an additional 48 h in the presence of γ-interferon (γ-IFN). The cells are then cultured at 39 C without γ-IFN in a plastic petri dish and/or soft agar gel for 2 weeks. Continuous cell growth in petri dishes and colony formation in soft agar is observed.

The cells incubated with the liver S 9 fraction from AOM-treated wild type mice should grow continuously in a petri dish at 39 C without γ-IFN, which is a sign of transformation. Anchorage-independent growth in soft agar gel, another sign of cell transformation, is also confirmed. The rate of transformation is determined by counting the number of colonies with a colony analyzer (OMNICON3800, Biologics, Gainesville, Va., United States of America). If the cells do not show any sign of transformation, the experiment is repeated with the S 9 fractions obtained from mouse liver following treatment with larger amounts of AOM (20 mg/kg, I.P.). The success of this unique cell transformation assay system provides an evaluation of the effect of the target gene on cell transformation.

Weak mutagenicity of the AOM is addressed as necessary. If even a higher dose of AOM (20 mg/kg, I.P.) does not cause cell transformation, one approach is to utilize a stronger mutagen such as benzo[a]pyrene (B[a]P). B[a]P is a very strong mutagen, but bioactivation is required in order to exhibit its mutagenicity. Ioannides, C., Parkinson, C. and Parke, D. V., Xenobiotica 1981; 11:701-8.

### Example 14

### Effect of Ang II Receptor Expression on Cytochrome P450 Expression in Mouse Hepatocytes

This Example employs four types of primary cultured hepatocytes prepared from the livers of male AT₂-KO and AT₁ₐ-KO mice and their corresponding wild type mice. Congenic AT₁ₐ KO mice (AT₁ₐ^{-/-}) with a C57BL/6J background are available from Drs. T. Matsusaka's and I. Ichikawa's laboratory, Department of Pediatrics, Vanderbilt University, Nashville, Tenn., United States of America. The AT₁ₐ receptor is the major isoform of the AT₁ receptors, and AT₁ₐ-KO mice exhibit significantly low systolic blood pressure. Ito, M., et al., Proc Natl Acad Sci USA. 1995; 92:3521-5; Sugaya, T., et al., J Biol Chem. 1995; 270:18719-22. AT₁ₐ-KO mice do not show any pressor responses to infused Ang II. Ito, M., et al., Proc Natl Acad Sci USA. 1995; 92:3521-5.

Both AT₁ₐ-KO and AT₂-KO mice and their corresponding control wild type mice are selected from among littermates after genotyping by Southern blot analysis. Hepatocytes are prepared by established methods. Freshney, R. I., Culture of epithelial cells. Wiley-Liss, Inc., 1992, pp. 197-223. Cytochrome P450 expression levels in primary cultured hepatocytes have been shown to decrease with time (Woodcroft, K. J. and Novak, R. F., Drug Metab Dispos. 1998; 26:372-378), so this experiment is conducted using primary cultured cells of passage 0 within 3 days.

The cells described above are individually stimulated with three or four prototypical cytochrome P450 inducers, 50 μM β-naphthoflavone or 50 μM 3-methylcholanthrene for CYP1A1 and 1A2 and 50 mM ethanol and 300 mM pyrazine for CYP2E1, in the presence or absence of 100 nM Ang II in culture medium. Changes in cytochrome P450 mRNA and protein levels are estimated by RT-PCR and Western blot analysis, respectively. Changes in CYP1A1, 1A2 and 2E1 activities are also determined with ethoxyresorufin, methoxyresorufin and chlorzoxazone as substrates, respectively. If the involvement of Ang II and the AT₁ and/or AT₂ receptor expressions in cytochrome P450 protein induction is observed, the roles of the subtype-specific Ang II receptors are confirmed by subtype-specific receptor blockers, losartan for the AT₁ receptor and PD123319 for the AT₂ receptor. Whether the Ang II effect on cytochrome P450 protein expression is transcriptional or posttranscriptional is also evaluated in the presence or absence of actinomycin D (5 μg/ml, transcription blocker) and separately in the presence or absence of cycloheximide (5 μg/ml, protein synthesis blocker).

The hepatocytes prepared from both control wild type mice should show increases in all three cytochrome P450 mRNA and protein levels in response to each inducer treatment, except for treatment with ethanol, which should only increase the CYP2E1 protein level. The hepatocytes prepared from AT₂-KO mouse liver might not respond to the CYP2E1 inducers pyrazine and ethanol as observed with the inducer AOM herein above. Responses to the CYP1A1 and 1A2 inducer -naphthoflavone in AT₂-KO hepatocytes can also be smaller than in the wild type hepatocytes. If the cytochrome P450 induction in hepatocytes prepared from AT₁ₐ-KO mouse liver is also lower than that in the hepatocytes prepared from the corresponding control wild type mouse liver, an involvement of the AT₁ receptor in hepatic cytochrome P450 1A1, 1A2 and 2E1 inductions is suggested.

The addition of Ang II to the culture medium is expected to show some positive effect on the cytochrome P450 induction. If the addition of Ang II to wild type cells does not show any effect but AT₂^{-/y} hepatocytes exhibit a lower inducibility of the cytochrome P450s, the AT₂ receptor is considered to be constitutively active and positively regulate cytochrome P450 expression.

CYP1A1 and 2E1 have been shown to be regulated by a cross-repressive regulation mechanism through H₂O₂ as a common mediator. Morel, Y., de Waziers, I. and Barouki, R., Mol Pharmacol. 2000; 57:1158-64. CYP2E1 expression is also regulated by its own protein expression (autoregulation). Morel, Y., de Waziers, I. and Barouki, R., Mol Pharmacol. 2000; 57:1158-64. Ang II increases intracellular reactive oxygen through the AT₁ receptor. Zafari, A. M., et al., Hypertension 1998; 32:488-95. The experimental design with hepatocytes from Ang II receptor-KO mice of two types and the corresponding wild type hepatocytes should simplify the complex response. If interference by an Ang II-induced reactive oxygen species is suspected, an antioxidant such as N-acetyl-L-cysteine or H₂O₂ reductase (catalase) is added.

### Example 15

### Determination of the Roles of Nuclear Ang II Receptors in Cytochrome P450 Expression in Mouse Hepatocytes

Several recent studies have characterized nuclear localization of Ang II receptors in several tissues. Eggena, P. et al., J Hypertens 1996; 14:961-8; Harris, R. C., Am J Med Sci. 1999; 318:374-9; Lu, D., et al., Endocrinology 1998; 139:365-75. In isolated hepatic nuclei we have detected fairly high levels of Ang II-AT₁ and AT₂ binding sites as described above. Although gene induction activated by G protein-coupled receptors often involves translocation of cytosolic kinases such as MAPK to the nucleus followed by activation of transcription factors, the newly detected receptors along with endocytosed ligands could conceivably be direct players in the gene activation story. An in vitro mRNA transcription assay is employed to answer this question.

Intact nuclei from the AT₂-KO, AT₁ₐ-KO are prepared, and corresponding wild type mice livers (description of animals is the same as described herein above) by differential centrifugation as described elsewhere. Nuclei in suspension are treated with Ang II (1-100 nM) in the presence or absence of either losartan (0.1-1 μM) or PD123319 (0.1-1 μM) for 30 min at 25 C in transcription buffer (Promega, Madison, Wis., United States of America). The reaction mixture is further incubated at 37 C for 60 min with an in vitro transcription system (Promega). The levels of total RNA and cytochrome P450 mRNA transcription are determined by slot-blot hybridization techniques with specific mRNA riboprobes. The complementary DNA clones for human CYP1A1, 1A2 and 2E1 are available from Dr. F. P. Guengerich, Department of Biochemistry, Vanderbilt University, Nashville, Tenn. These cDNA clones have been shown to recognize the mouse orthologs.

Ang II should dose-responsively increase cytochrome P450 mRNA transcription in the hepatocyte nuclei prepared from both control wild type mice. However, either AT₂-KO or AT₁ₐ-KO mice hepatocyte nuclei could show very weak responses to Ang II. Ang II has been shown to increase the transcription of mRNA of the protooncogene c-myc, platelet-derived growth factor, insulin-like growth factor, renin and angiotensinogen in rat hepatocyte nuclei (Eggena, P., et al., J Hypertens 1996; 14:961-8), although subtype-specific receptor function has not been studied rigorously. Nuclear translocation of the Ang II-AT₁ receptor in neuronal cells in association with an Ang II-stimulated increase in tyrosine hydroxylase transcription has also been reported. Lu, D., et al., Endocrinology 1998; 139:365-75. The nuclear translocation of the AT₁ receptor and/or AT₂ receptor in mice hepatocytes is clarified by an immunofluorescence tracking technique with AT₁ₐ and AT₂ receptor antibodies. Polyclonal AT₁ receptor antibodies have been generated by Rakugi et al. (Department of Medicine, Osaka University, Japan). Rakugi, H., et al., Hypertens Res. 1997; 20:51-5. The usefulness of these antibodies has been confirmed by both Western blot analysis and immunohistochemistry.

Weak transcriptional regulation of cytochrome P450 expression in Ang II-treated hepatocyte nuclei can be addressed by increasing the assay scale. A larger sample is also analyzed.

### Example 16

### Characterization of Post-Receptor Signaling Components in Ang II Receptor-Regulated Hepatic Cytochrome P450 Expression

Induction of cytochrome P450 enzymes is regulated by both transcriptional and posttranscriptional mechanisms. Whitlock, & Denison, in: Cytochrome P450: Structure, Mechanism and Biochemistry (2nd ed.), (Montellano, ed.), Plenum Press, New York, N.Y. (1995). CYP1A1 and 1A2 inductions are mainly regulated by a transcriptional mechanism through both the arylhydrocarbon (Ah) receptor and the Ah receptor nuclear translocator (Arnt). Whitlock, (1999) Ann. Rev. Pharmacol. Toxicol. 39:103-25; Xu et al., (2000) Chem Biol Interact. 124:173-89. CYP2E1 induction is mainly regulated by a posttranscriptional mechanism through mRNA and protein stabilization. Whitlock, & Denison, in: Cytochrome P450: Structure, Mechanism and Biochemistry (2nd ed.), (Montellano, ed.), Plenum Press, New York, N.Y. (1995). Since substantial increases in all three cytochrome P450 proteins by AOM treatment within a short time are observed as presented herein above, whether this increase is due to transcriptional or posttranscriptional regulation is clarified by measuring their mRNA and protein levels. If AOM-dependent cytochrome P450 enzyme induction is suggested to be transcriptional, the effect of Ang II receptor status on transcription efficiencies of cytochrome P450 proteins is evaluated by a nuclear run-on assay. The procedure for the nuclear run-on assay is the standard method described by Reiners et al., (1997) Mol. Carcinog. 19:91-100.

The effect of Ang II receptor status on the expression of the Ah receptor and Arnt protein is studied in hepatocytes derived from AT₁ₐ-KO and AT₂-KO mice and their corresponding wild type mice. The levels of both transcription factors are determined by semi-quantitative RT-PCR with an internal standard of GAPDH expression and/or Western blot analysis with their specific antibodies. In addition, the efficiency of nuclear translocation of the Ah receptor protein is also determined.

The molecular mechanism by which the AT₂ receptor positively regulates the three cytochrome P450 protein inductions, based on the known induction mechanism of CYP1A1, is evaluated. Although CYP2E1 induction can be regulated by posttranscriptional and posttranslational mechanisms, there might be a unified connection among all three cytochrome P450 protein inductions in hepatocytes. If an association of Ah receptor and/or Arnt protein expressions or Ah receptor nuclear translocation with Ang II receptor status is observed, how the Ang II receptor-mediated signal(s) is associated with Ah receptor activation or nuclear translocation is further clarified by Western blot analysis of the Ang II post-receptor signaling components and components in the Ah receptor complex.

If all three cytochrome P450 proteins are regulated by posttrancriptional and posttranslational mechanisms, the factor(s) that influences the mRNA or protein stability is/are determined. A candidate for this factor could be a proteasome complex. Degradation of a protein through the ubiquitin-proteasome pathway has been shown in the regulation of many cellular proteins, including transcription factors and a cytosolic steroid receptor (Hershko & Ciechanover, (1998) Annu Rev Biochem. 67:425-79). This proteasomal degradation of a protein involves ubiquitination of the target protein. The highly ubiquitinated protein is rapidly degraded by the 26S proteasome. The involvement of ubiquitin-proteasome-dependent degradation of the cytochrome P450 proteins is evaluated. A 26S proteasome inhibitor such as MG132 or lactacystein is utilized in order to specify proteasome-specific degradation. If cytochrome P450 proteins are degraded by this pathway, and attenuation of this pathway occurs in the stimulated hepatocytes, a part of the posttranslational regulation of cytochrome P450 proteins is indicated.

### Example 17

### Effect of Host-Angiotensin II Receptor Status on Xenografted Tumor Growth in Mice

Lewis lung carcinoma (LLC) cells were suspended in 0.6% solution of agarose at a concentration of 2×10⁶ viable cells/ml. The suspension was loaded into tuberculin syringes and allows gelling at 4° C. for 2 min. fifty microliters of cell suspension (1×10⁵ viable cells) was injected subcutaneously using a 25-gauge needle in both flanks. In PD 123,319 treatment experiments, mice were pre-treated with PD 123,319 (30 mg/kg/day in drinking water) for a day before tumor implantation. The size of the tumor was determined by direct measurement of tumor dimensions. The volume was calculated according to the equation (V=[L×W2]×0.5), where V=volume, L=length, and W=width. See FIG. 7 (solid circles, Ed wild, N=6; open circles, AT2 KO, N=5).

### Statistical Analysis Employed in Examples 7-17

Data is presented as means ±S.E. The significance of differences between control and experimental groups (minimum number=6-7) are evaluated using a one-way analysis of variance (ANOVA) with Student-Newman-Keuls test. P<0.05 is considered statistically significant.

### REFERENCES

The references listed below as well as all references cited in the specification are incorporated herein by reference to the extent that they supplement, explain, provide a background for or teach methodology, techniques and/or compositions employed herein. All cited patents and publications referred to in this application are herein expressly incorporated by reference. Also expressly incorporated herein by reference are the contents of all citations of GenBank accession numbers, LocusID, and other computer database listings.

Allen, J. I. Molecular biology of colon polyps and colon cancer. Semin Surg Oncol. 1995; 11:399-405.

Armstrong, R. N. Enzyme-catalyzed detoxification reactions: mechanisms and stereochemistry. CRC Crit Rev Biochem. 1987; 22:39-88.

Ausubel, F. M., Brent, R., Kingston, R. E., Moore, D. D., Seidman, J. G., Smith, J. A. and Struhl, K. Short Protocols in Molecular Biology. . New York: Greene Publishing Associates and John Wiley & Sons, 1992.

Badawi, A., Stern, S., Lang, N. and Kadlubar, F. Cytochrome P-450 and acetyltransferase expression as biomarkers of carcinogen-DNA adduct levels and human cancer susceptibility. Prog Clin Biol Res. 1996; 395:109-140.

Blume, A., Herdegen T. and Unger T. (1999) Angiotensin peptides and inducible transcription factors. J Mol Med, 77, 339-57.

Bock, K. W. Roles of UDP-glucuronosyltransferases in chemical carcinogenesis. Crit Rev Biochem Mol Biol. 1991; 26:129-150.

Boland, C. Malignant tumors of the colon. In: Textbook of gastroenterology (2nd ed.), edited by Yamada, T. J B Lippincott, Philadelphia: 1995, pp.1967-2026.

Carey, R. M., Wang, Z. Q. and Siragy, H. M. Role of the angiotensin type 2 receptor in the regulation of blood pressure and renal function. Hypertension. 2000; 35:155-163.

Chen, L., Re, R. N., Prakash, O. and Mondal, D. Angiotensin-converting enzyme inhibition reduces neuroblastoma cell growth rate. Proc Soc Exp Biol Med. 1991; 196:280-283.

Chida, M., Yokoi, T., Fukui, T., Kinoshita, M., Yokota, J. and Kamataki, T. Detection of three genetic polymorphisms in the 5′-flanking region and intron 1 of human CYP1A2 in the Japanese population. Jpn J Cancer Res. 1999; 90:899-902.

Chiu, A., Herblin, W., McCall, D., Ardecky, R., Carini, D., Dunicia, J., Pease, L., Wong, P., Wexler, R., Johnson, A. and Timmermans, P. Identification of angiotensin II receptor subtypes. Biochem Biophys Res Commun. 1989; 165:196-203.

Csikos, T., Gallinat, S. and Unger, T. Extrarenal aspects of angiotensin 11 function. Eur J Endocrinol. 1997; 136:349-358.

D'Abaco, G. M., Whitehead, R. H. and Burgess, A. W. Synergy between Apc min and an activated ras mutation is sufficient to induce colon carcinomas. Mol Cell Biol. 1996; 16:884-91.

Dimmeler, S., Rippmann, V., Weiland, U., Haendeler, J. and Zeiher, A. M. Angiotensin II induces apoptosis of human endothelial cells. Circ Res. 1997; 81:970-976.

Dudley, D. T., Hubbell, S. E. and Summerfelt, R. M. Characterization of angiotensin II (AT₂) binding sites in R3T3 cells. Mol Pharmacol. 1991; 40:360-367.

Eggena, P., Zhu, J. H., Sereevinyayut, S., Giordani, M., Clegg, K., Andersen, P. C., Hyun, P. and Barrett, J. D. Hepatic angiotensin II nuclear receptors and transcription of growth-related factors. J Hypertens. 1996; 14:961-8.

Egidy, G., Friedman, J., Viswanathan, M., Wahl, L. M. and Saavedra, J. M. CGP-42112 partially activates human monocytes and reduces their stimulation by lipopolysaccharides. Am J Physiol. 1997; 273:C826-33.

Erdman, S., We H., Hixson L., Ahnen D. and Gerner E. (1997) Assessment of mutations in Ki-ras and p53 in colon cancers from azoxymethane- and dimethylhydrazine-treated rats. Molecular Carinogenesis, 19, 137-144.

European Patent No. EP174162

Fernandez, L. A., Twickler, J. and Mead, A. Neovascularization produced by angiotensin II. J Lab Clin Med. 1985; 105:141-145.

Fiala, E. S., Joseph C., Sohn O. S., el-Bayoumy K. and Reddy B. S. (1991) Mechanism of benzylselenocyanate inhibition of azoxymethane-induced colon carcinogenesis in F344 rats. Cancer Res, 51, 2826-30.

Freshney, R. I. Culture of epithelial cells. Wiley-Liss, Inc., 1992, pp. 197-223.

Friis, S., Sorensen H. T., Mellemkjaer L., McLaughlin J. K., Nielsen G. L., Blot W. J. and Olsen J. H. (2001) Angiotensin-converting enzyme inhibitors and the risk of cancer: a population-based cohort study in Denmark. Cancer, 92, 2462-70.

Gallinat, S., Busche, S., Raizada, M. K. and Sumners, C. The angiotensin II type 2 receptor: an enigma with multiple variations. Am J Physiol Endocrinol Metab. 2000; 278:E357-74.

Giardello, F. Gastrointestinal polyposis syndromes and hereditary nonpolyposis colorectal cancer. In: Gastrointestinal cancer: biology, diagnosis and therapy, edited by Rustgi, A. Lippincott-Raven, Philadelphia: 1995, pp. 367-377

Giovannucci, E., Colditz, G. A., Stampfer, M. J., Hunter, D., Rosner, B. A., Willett, W. C. and Speizer, F. E. A prospective study of cigarette smoking and risk of colorectal adenoma and colorectal cancer in U.S. women. J Natl Cancer Inst. 1994; 86:192-199.

Giovannucci, E., Rimm, E. B., Stampfer, M. J., Colditz, G. A., Ascherio, A., Kearney, J. and Willett, W. C. A prospective study of cigarette smoking and risk of colorectal adenoma and colorectal cancer in U.S. men. J Natl Cancer Inst. 1994; 86:183-191.

Glauert, H. P. and Bennink, M. R. Metabolism of 1,2-dimethylhydrazine by cultured rat colon epithelial cells. Nutr Cancer. 1983; 5:78-86.

Goldszer, R. C., Lilly, L. S. and Solomon, H. S. Prevalence of cough during angiotensin-converting enzyme inhibitor therapy. Am J Med. 1988; 85:887.

Grady, E. F., Sechi, L. A., Griffin, C. A., Schambelan, M. and Kalinyak, J. E. Expression of AT₂ receptors in the developing rat fetus. J Clin Invest. 1991; 88:921-933.

Grove, A. D., Kessler, F. K., Metz, R. P. and Ritter, J. K. Identification of a rat oltipraz-inducible UDP-glucuronosyltransferase (UGTIA7) with activity towards benzo(a)pyrene-7,8-dihydrodiol. J Biol Chem. 1997; 272:1621-1627.

Guengerich, F. P. and Shimada, T. Oxidation of toxic and carcinogenic chemicals by human cytochrome P-450 enzymes. Chem Rese Toxicol. 1991; 4:391-407

Guengerich, F. P. Enzymatic oxidation of xenobiotic chemicals. Crit Rev Biochem Mol Biol. 1990; 25:97-153.

Guengerich, F. P., Kim, D. H. and Iwasaki, M. Role of human cytochrome P-450 IIE1 in the oxidation of many low molecular weight cancer suspects. Chem Res Toxicol. 1991; 4:168-179.

Hamilton, S. R., Sohn O. S. and Fiala E. S. (1988) Inhibition by dietary ethanol of experimental colonic carcinogenesis induced by high-dose azoxymethane in F344 rats. Cancer Res, 48, 3313-8.

Harris, R. C. Potential mechanisms and physiologic actions of intracellular angiotensin II. Am J Med Sci. 1999; 318:374-9.

Hayashi, S., Watanabe, J. and Kawajiri, K. Genetic polymorphisms in the 5′-flanking region change transcriptional regulation of the human cytochrome P450IIE1 gene. J Biochem (Tokyo). 1991; 110:559-565.

Hayashi, S., Watanabe, J., Nakachi, K. and Kawajiri, K. Genetic linkage of lung cancer-associated MspI polymorphisms with amino acid replacement in the heme binding region of the human cytochrome P450IA1 gene. J Biochem (Tokyo). 1991; 110:407-11.

Heilbrun, L. K., Nomura, A., Hankin, J. H. and Stemmermann, G. N. Diet and colorectal cancer with special reference to fiber intake. Int J Cancer. 1989; 44:1-6.

Hein, L., Barsh, G. S., Pratt, R. E., Dzau, V. J. and Kobilka, B. K. Behavioural and cardiovascular effects of disrupting the angiotensin II type-2 receptor in mice. Nature. 1995; 377:744-7.

Hershko, A. and Ciechanover, A. The ubiquitin system. Annu Rev Biochem. 1998; 67:425-79

Hii, S. I., Nicol D. L., Gotley D. C., Thompson L. C., Green M. K. and Jonsson J. R. (1998) Captopril inhibits tumour growth in a xenograft model of human renal cell carcinoma. Br J Cancer, 77, 880-883.

Horiuchi, M., Akishita, M. and Dzau, V. J. Recent progress in angiotensin II type 2 receptor research in the cardiovascular system. Hypertension. 1999; 33:613-621.

Howe, G. R., Benito, E., Castelleto, R., Cornee, J., Esteve, J., Gallagher, R. P., Iscovich, J. M., Deng-ao, J., Kaaks, R., Kune, G. A. and et al. Dietary intake of fiber and decreased risk of cancers of the colon and rectum: evidence from the combined analysis of 13 case-control studies. J Natl Cancer Inst. 1992; 84:1887-1896.

Hu, et al., Comparative Studies of the Angiogenic Activity of Vasoactive Intestinal Peptide, Endothelins-1 and -3 and Angiotensin II in a Rat Sponge Model, Br J Pharmacol (1996); 117: 545-551.

Ichiki, T., Labosky, P. A., Shiota, C., Okuyama, S., Imagawa, Y., Fogo, A., Niimura, F., Ichikawa, I., Hogan, B. L. and Inagami, T. Effects on blood pressure and exploratory behaviour of mice lacking angiotensin II type-2 receptor. Nature. 1995; 377:748-750.

Ilyas, M., Straub, J., Tomlinson, I. P. and Bodmer, W. F. Genetic pathways in colorectal and other cancers. Eur J Cancer. 1999; 35:1986-2002.

Ioannides, C., Parkinson, C. and Parke, D. V. Activation of benzo(a)pyrene and 2-acetamidofluorene to mutagens by microsomal preparations from different animal species: role of cytochrome P-450 and P-448. Xenobiotica. 1981; 11:701-8.

Ito, M., Oliverio, M. I., Mannon, P. J., Best, C. F., Maeda, N., Smithies, 0. and Coffman, T. M. Regulation of blood pressure by the type 1A angiotensin 11 receptor gene. Proc Natl Acad Sci USA. 1995; 92:3521-5.

Jacoby, R. F., Llor, X., Teng, B. B., Davidson, N. O. and Brasitus, T. A. Mutations in the K-ras oncogene induced by 1,2-dimethylhydrazine in preneoplastic and neoplastic rat colonic mucosa. J Clin Invest. 1991; 87:624-30.

Jakobsson, K., Albin, M. and Hagmar, L. Asbestos, cement, and cancer in the right part of the colon. Occup Environ Med. 1994; 51:95-101.

Japanese Patent No. 3-1671981

Japanese Patent No. 62-270533

Japanese Patent No. 64-5497

Japanese Patent No. 64-83096

Jin, X. -H., Wang, Z. -Q., Siragy, H. M., Guerrant, R. L. and Carey, R. M. Regulation of jejunal sodium and water absorption by angiotensin subtype receptors. Am J Physiol. 1998; 275:R515-R523.

Johnston, C. I. (1993) Angiotensin-converting enzyme inhibitors. In Robertson, J. I. S. and Nicholls, M. G. (eds), The renin-angiotensin system. Vol. 2, Pathophysiology, Therapeutics. Mosby, London, pp. 87.1-87.12.

Johnston, C. I., Jackson, B., Cubela, R., Larmour, I. and Arnolda, L. Evaluation of angiotensin converting enzyme (ACE) in the pharmacokinetics and pharmacodynamics of ACE inhibitors. J Cardiovasc Pharmacol. 1986; 8:S9-S14.

Kambayashi, Y., Bardhan, S., Takahashi, K., Tsuzuki, S., Inui, H., Hamakubo, T. and Inagami, T. Molecular cloning of a novel angiotensin 11 receptor isoform involved in phosphotyrosine phosphatase inhibition. J Biol Chem. 1993; 268:24543-24546.

Kardum, D., Huskic, J., Fabijanic, D., Banic, M., Buljevac, M., Kujundzic, M. and Loncar, B. Activity of serum angiotensin-converting enzyme as a tumour marker of hepatocellular carcinoma. Eur J Gastroenterol Hepatol. 1999; 11:1209-1213.

Kawajiri, K., Nakachi, K., Imai, K., Yoshii, A., Shinoda, N. and Watanabe, J. Identification of genetically high risk individuals to lung cancer by DNA polymorphisms of the cytochrome P4501A1 gene. FEBS Lett. 1990; 263:131-3.

Kern, S. and Kinzler, K. Molecular genetics of colorectal carcinoma. In: Gastrointestinal cancer: biology, diagnosis and therapy, edited by Rustgi, A. Lippincott-Raven, Philadelphia: 1995, pp. 413-422.

Kiss, I., Sandor, J., Pajkos, G., Bogner, B., Hegedus, G. and Ember, I. Colorectal cancer risk in relation to genetic polymorphism of cytochrome P450 1A1, 2E1, and glutathione-S-transferase M1 enzymes. Anticancer Res. 2000; 20:519-522.

Kleinmann, et al., The Good Side of Side Effects: Captopril Blocks Vessel and Tumor Growth; J. Clin. Invest (August 1996); 98:599.

Koop, D. R. and Tierney, D. J. Multiple mechanisms in the regulation of ethanol-inducible cytochrome P45011E1. Bioessays. 1990; 12:429-435.

Landis, S. H., Murray, T., Bolden, S. and Wingo, P. A. Cancer statistics, 1999. CA Cancer J Clin. 1999; 49:8-31.

Lang, N., Butler M., Massengill J., Lawson M., Stotts R., Hauer-Jensen M. and Kadlubar F. (1994) Rapid metabolic phenotypes for acetyltransferase and cytochrome P4501A2 and putative exposure to food-borne heterocyclic amines increase the risk for colorectal cancer or polyps. Cancer Epidemiol Biomarker Prev, 3, 675-682.

Lang, N., Butler, M., Massengill, J., Lawson, M., Stotts, R., Hauer-Jensen, M. and Kadlubar, F. Rapid metabolic phenotypes for acetlytransferase and cytochrome P4501A2 and putative exposure to food-borne heterocyclic amines increase the risk for colorectal cancer or polyps. Cancer Epidemiol Biomarker Prev. 1994; 3:675-682.

Lashner, B. A. and Epstein, S. S. Industrial risk factors for colorectal cancer. Int J Health Serv. 1990; 20:459-483.

le Noble, F. A., Kessels-van Wylick, L. C., Hacking, W. J., Slaaf, D. W., oude Egbrink, M. G. and Struijker-Boudier, H. A. The role of angiotensin II and prostaglandins in arcade formation in a developing microvascular network. J Vasc Res. 1996; 33:480-488.

Lever, A. F., Hole, D. J., Gillis, C. R., McCallum, I. R., McInnes, G. T., MacKinnon, P. L., Meredith, P. A., Murray, L. S., Reid, J. L. and Robertson, J. W. Do inhibitors of angiotensin-l-converting enzyme protect against risk of cancer? Lancet. 1998; 352:179-184.

Levy, B. I., Benessiano, J., Henrion, D., Caputo, L., Heymes, C., Duriez, M., Poitevin, P. and Samuel, J. L. Chronic blockade of AT₂-subtype receptors prevents the effect of angiotensin II on the rat vascular structure. J Clin Invest. 1996; 98:418-425.

Lichtenstein, P., Holm, N. V., Verkasalo, P. K., Iliadou, A., Kaprio, J., Koskenvuo, M., Pukkala, E., Skytthe, A. and Hemminki, K. Environmental and heritable factors in the causation of cancer—analyses of cohorts of twins from Sweden, Denmark, and Finland. N Engl J Med. 2000; 343:78-85.

Lindholm, L. H., Anderson H., Ekbom T., Hansson L., Lanke J., Dahlof B., de Faire U., Forsen K., Hedner T., Linjer E., Schersten B., Wester P. and Moller T. (2001) Relation between drug treatment and cancer in hypertensives in the Swedish Trial in Old Patients with Hypertension 2: a 5-year, prospective, randomised, controlled trial. Lancet, 358, 539-44.

Lokuta, A. J., Cooper, C., Gaa, S. T., Wang, H. E. and Rogers, T. B. Angiotensin II stimulates the release of phospholipid-derived second messengers through multiple receptor subtypes in heart cells. J Biol Chem. 1994; 269:4832-4838.

Longnecker, M. P., Orza, M. J., Adams, M. E., Vioque, J. and Chalmers, T. C. A meta-analysis of alcoholic beverage consumption in relation to risk of colorectal cancer. Cancer Causes Control. 1990; 1:59-68.

Lu, D., Yang, H., Shaw, G. and Raizada, M. K. Angiotensin l-induced nuclear targeting of the angiotensin type 1 (AT1) receptor in brain neurons. Endocrinology. 1998; 139:365-75.

Lu, D., Yu, K. and Raizada, M. K. Retrovirus-mediated transfer of an angiotensin type I receptor (AT1-R) antisense sequence decreases AT1-Rs and angiotensin II action in astroglial and neuronal cells in primary cultures from the brain. Proc Natl Acad Sci USA. 1995; 92:1162-1166.

Lyall, F., Dornan, E. S., McQueen, J., Boswell, F. and Kelly, M. Angiotensin II increases proto-oncogene expression and phosphoinositide turnover in vascular smooth muscle cells via the angiotensin II AT1 receptor. J Hypertens. 1992; 10:1463-1469.

Lyall, F., et al., Angiotensin II Increases Proto-oncogene Expression and Phosphoinositide Turnover in Vascular Smooth Muscle Cells via the Angiotensin II AT1 Receptor, J Hypertens (1996); 10: 1463-1469.

Masmoudi, A., Labourdette, G., Mersel, M., Huang, F. L., Huang, K. P., Vincendon, G. and Malviya, A. N. Protein kinase C located in rat liver nuclei. Partial purification and biochemical and immunochemical characterization. J Biol Chem. 1989; 264:1172-9.

Matsusaka, T., Fogo A. and Ichikawa I. (1997) Targeting the genes of angiotensin receptors. Semin Nephrol, 17, 396-403.

Migdalof, B. H., Antonaccio, M. J., McKinstry, D. N., Singhvi, S. M., Lan, S. J., Egli, P. and Kripalani, K. J. Captopril: pharmacology, metabolism and disposition. Drug Metab Rev. 1984; 15:841-869.

Miyake, S., Makimura, M., Kanegae, Y., Harada, S., Sato, Y., Takamori, K., Tokuda, C. and Saito, I. Efficient generation of recombinant adenoviruses using adenovirus DNA-terminal protein complex and a cosmid bearing the full-length virus genome. Proc Natl Acad Sci USA. 1996; 93:1320-4.

Morel, Y., de Waziers, I. and Barouki, R. A repressive cross-regulation between catalytic and promoter activities of the CYP1A1 and CYP2E1 genes: role of H(2)O(2). Mol Pharmacol. 2000; 57:1158-64.

Mukoyama, M., Nakajima, M., Horiuchi, M., Sasamura, H., Pratt, R. E. and Dzau, V. J. Expression cloning of type 2 angiotensin II receptor reveals a unique class of seven-transmembrane receptors. J Biol Chem. 1993; 268:24539-24542.

Munzenmair, et al. Opposing Actions of Angiotensin II on Microvascular Growth and Arterial Blood Pressure, Hypertension (1996); 27: 760-765.

Nagao, M. and Sugimura, T. Carcinogenic factors in food with relevance to colon cancer development. Mutat Res. 1993; 290:43-51.

Nakajima, et al., The Angiotensin II Type 2 (AT₂) Receptor Antagonizes the Growth Effects of the AT₁ Receptor: Gain-of-Function Study Using Gene Transfer. Proc Natl Acad Sci USA (1995); 92: 10663-10667.

Nakajima, M., Hutchinson H. G., Fujinaga M., Hayashida W., Morishita R., Zhang L., Horiuchi M., Pratt R. E. and Dzau V. J. (1995) The angiotensin II type 2 (AT₂) receptor antagonizes the growth effects of the AT₁ receptor: gain-of-function study using gene transfer. Proc Natl Acad Sci USA, 92, 10663-10667.

Neyses, L., Nouskas, J., Luyken, J., Fronhoffs, S., Oberdorf, S., Pfeifer, U., Williams, R. S., Sukhatme, V. P. and Vetter, H. Induction of immediate-early genes by angiotensin II and endothelin-1 in adult rat cardiomyocytes. J Hypertens. 1993; 11:927-934.

Oertle, M. and Muller, J. Two types of cytochrome P-450(11 beta) in rat adrenals: separate regulation of gene expression. Mol Cell Endocrinol. 1993; 91:201-209.

Otani, A., Takagi H., Suzuma K. and Honda Y. (1998) Angiotensin II potentiates vascular endothelial growth factor-induced angiogenic activity in retinal microcapillary endothelial cells. Circ Res, 82, 619-28.

Papanikolaou, A., Wang Q. -S., Delker D. A. and Rosenberg D. W. (1998) Azoxymethane-induced colon tumors and aberrant crypt foci in mice of different genetic susceptibility. Cancer Lett, 130, 29-34.

Peach, M. Molecular actions of angiotensin. Biochem Pharmacol. 1981; 30:2745-2751.

Pegg, A. E. (1984) Methylation of the 06 position of guanine in DNA is the most likely initiating event in carcinogenesis by methylating agents. Cancer Invest, 2, 223-231

Peter, R., Bocker, R., Beaune, P. H., Iwasaki, M., Guengerich, F. P. and Yang, C. S. Hydroxylation of chlorzoxazone as a specific probe for human liver cytochrome P-450IIE1. Chem Res Toxicol. 1990; 3:566-573.

Potter, J. D. Colorectal cancer: molecules and populations. J Natl Cancer Inst. 1999; 91:916-932.

Potter, J. D., Slattery, M. L., Bostick, R. M. and Gapstur, S. M. Colon cancer: a review of the epidemiology. Epidemiol Rev. 1993; 15:499-545.

Rakugi, H., Okamura, A., Kamide, K., Ohishi, M., Sasamura, H., Morishita, R., Higaki, J. and Ogihara, T. Recognition of tissue- and subtype-specific modulation of angiotensin II receptors using antibodies against AT1 and AT2 receptors. Hypertens Res. 1997; 20:51-5.

Reddy, M. K., Baskaran, K. and Molteni, A. Inhibitors of angiotensin-converting enzyme modulate mitosis and gene expression in pancreatic cancer cells. Proc Soc Exp Biol Med. 1995; 210:221-226.

Reiners, J. J., Jr., Jones, C. L., Hong, N., Clift, R. E. and Elferink, C. Downregulation of aryl hydrocarbon receptor function and cytochrome P450 1A1 induction by expression of Ha-ras oncogenes. Mol Carcinog. 1997; 19:91-100.

Rogers, K. J. and Pegg A. E. (1977) Formation of 06-methylguanine by alkylation of rat liver, colon, and kidney DNA following administration of 1,2-dimethylhydrazine. Cancer Res, 37, 4082-7.

Rosenberg, D. W. and Mankowski, D. C. Induction of cyp2e-1 protein in mouse colon. Carcinogenesis. 1994; 15:73-78.

Rosenberg, D. W. Dietary modulation of cytochrome P450 in the small intestinal epithelium. Pharmacology. 1991; 43:36-46.

Rudolph, U., Finegold, M. J., Rich, S. S., Harriman, G. R., Srinivasan, Y., Brabet, P., Boulay, G., Bradley, A. and Birnbaumer, L. Ulcerative colitis and adenocarcinoma of the colon in G alpha i2-deficient mice. Nat Genet. 1995; 10:143-50.

Sadoshima, J. and Izumo, S. Signal transduction pathways of angiotensin II-induced c-fos gene expression in cardiac myocytes in vitro. Roles of phospholipid-derived second messengers. Circ Res. 1993; 73:424-438.

Sambrook, T., Fritsch, E. F. and Maniatis, T. Molecular Cloning: A Laboratory Manual. (2nd ed.). Plainview, N. Y.: Cold Spring Harbor Laboratory Press, 1989.

Sasaki, T., Irie-Sasaki, J., Horie, Y., Bachmaier, K., Fata, J. E., Li, M., Suzuki, A., Bouchard, D., Ho, A., Redston, M., Gallinger, S., Khokha, R., Mak, T. W., Hawkins, P. T., Stephens, L., Scherer, S. W., Tsao, M. and Penninger, J. M. Colorectal carcinomas in mice lacking the catalytic subunit of PI(3)Kgamma. Nature. 2000; 406:897-902.

Scheuer, D. A. and Perrone, M. H. Angiotensin type 2 receptors mediate depressor phase of biphasic pressure response to angiotensin. Am J Physiol. 1993; 264:R917-R923.

Senbonmatsu, T., Ichihara, S., Price, E., Jr., Gaffney, F. A. and Inagami, T. Evidence for angiotensin II type 2 receptor-mediated cardiac myocyte enlargement during in vivo pressure overload. J Clin Invest. 2000; 106:R1-5.

Shamsuddin, A. K. and Trump, B. F. Colon epithelium. II. In vivo studies of colon carcinogenesis. Light microscopic, histochemical, and ultrastructural studies of histogenesis of azoxymethane-induced colon carcinomas in Fischer 344 rats. J Natl Cancer Inst. 1981; 66:389-401.

Shamsuddin, A. K. Carcinoma of the large intestine: animal models and human disease. Human Path. 1986; 17:451-453.

Shankar, S. and Lanza, E. Dietary fiber and cancer prevention. Hematol Oncol Clin North Am. 1991; 5:25-41.

Shimada, T., Yun, C. H., Yamazaki, H., Gautier, J. C., Beaune, P. H. and Guengerich, F. P. Characterization of human lung microsomal cytochrome P-450 1A1 and its role in the oxidation of chemical carcinogenesis. Mol Pharmacol. 1992; 41:856-864.

Shivapurkar, N., Belinsky, S., Wolf, D., Tang, Z. and Alabaster, 0. Absence of p53 gene mutations in rat colon carcinomas induced by azoxymethane. Cancer Lett. 1995; 96:63-70.

Shivapurkar, N., Tang Z., Ferreira A., Nasim S., Garett C. and Alabaster O. (1994) Sequential analysis of K-ras mutations in aberrant crypt foci and colonic tumors induced by azoxymethane in Fischer-344 rats on high-risk diet. Carcinogenesis, 15, 775-8.

Siragy, H. M. and Carey, R. M. The subtype-2 (AT₂) angiotensin receptor regulates renal cyclic guanosine 3′, 5′-monophosphate and AT₁ receptor-mediated prostaglandin E₂ production in conscious rats. J Clin Invest. 1996; 97:1978-1982.

Sivaraman, L., Leatham, M., Yee, J., Wikens, L., Lau, A. and Le Marchand, L. CYP1A1 genetic polymorphisms and in situ colorectal cancer. Cancer Res. 1994; 54:3692-3695.

Sohn, O. S., Fiala E. S., Requeijo S. P., Weisburger J. H. and Gonzalez F. J. (2001) Differential effects of CYP2E1 status on the metabolic activation of the colon carcinogens azoxymethane and methylazoxymethanol. Cancer Res, 61, 8435-40.

Sohn, O. S., Ishizaki, H., Yang, C. S. and Fiala, E. S. Metabolism of azoxymethane, methylazoxymethanol and N-nitrosodimethylamine by cytochrome P450IIE1. Carcinogenesis. 1991; 12:127-131.

Soucek, P., Martin, M. V., Ueng, Y. F. and Guengerich, F. P. Identification of a common cytochrome P450 epitope near the conserved heme-binding peptide with antibodies raised against recombinant cytochrome P450 family 2 proteins. Biochemistry. 1995; 34:16013-16021.

Stahl, M., Bulpift C. J., Palmer A. J., Beevers D. G., Coles E. C. and Webster J. (2000) Calcium channel blockers, ACE inhibitors, and the risk of cancer in hypertensive patients: a report from the Department of Health Hypertension Care Computing Project (DHCCP). J Hum Hypertens, 14, 299-304.

Stellman, J. M. and Stellman, S. D. Cancer and the workplace. CA Cancer J Clin. 1996; 46:70-92.

Stoll, M., Steckelings, U. M., Paul, M., Bottari, S. P., Metzger, R. and Unger, T. The angiotensin AT2-receptor mediates inhibition of cell proliferation in coronary endothelial cells. J Clin Invest. 1995; 95:651-657.

Strassburg, C. P., Nguyen, N., Manns, M. P. and Tukey, R. H. UDP-glucuronosyltransferase activity in human liver and colon. Gastroenterology. 1999; 116:149-160.

Sugaya, T., Nishimatsu, S., Tanimoto, K., Takimoto, E., Yamagishi, T., Imamura, K., Goto, S., Imaizumi, K., Hisada, Y., Otsuka, A. and et al. Angiotensin II type 1a receptor-deficient mice with hypotension and hyperreninemia. J Biol Chem. 1995; 270:18719-22.

Sugimura, T., Nagao, M. and Wakabayashi, K. Carcinogenicity of food mutagens. Environ Health Perspect. 1996; 104 Suppl 3:429-433.

Takahashi, M., Nakatsugi, S., Sugimura, T. and Wakabayashi, K. Frequent mutations of the beta-catenin gene in mouse colon tumors induced by azoxymethane. Carcinogenesis. 2000; 21:1117-1120.

Tamura, M., Chen, Y. J., Howard, E. F., Tanner, M., Landon, E. J. and Myers, P. R. Lipopolysaccharides and cytokines downregulate the angiotensin II type 2 receptor in rat cardiac fibroblasts. Eur J Pharmacol. 1999; 386:289-295.

Tamura, M., Takagi, T., Howard, E. F., Landon, E. J., Steimle, A., Tanner, M. and Myers, P. R. Induction of angiotensin II subtype 2 receptor-mediated blood pressure regulation in synthetic diet-fed rats. J Hypertens. 2000; 18:1239-124.

Tamura, M., Utsunomiya, H., Nakamura, M. and Landon, E. J. Effect of dietary cardiac glycosides on blood pressure regulation in rats. Can J Physiol Pharmacol. 2000; 78:548-56.

Tamura, M., Wanaka, Y., Landon, E. J. and Inagami, T. Intracellular sodium modulates the expression of angiotensin II subtype 2 receptor in PC12W cells. Hypertension. 1999; 33:626-632.

Tanaka, M., Tsuchida, S., Imai, T., Fujii, N., Miyazaki, H., Ichiki, T., Naruse, M. and Inagami, T. Vascular response to angiotensin II is exaggerated through an upregulation of AT₁ receptor in AT₂ knockout mice. Bioch Biophys Res Commu. 1999; 258:194-198

Thomale, J., Engelbergs, J., Seiler, F. and Rajewsky, M. F. Monoclonal antibody-based quantification and repair analysis of specific alkylation products in DNA. In: Technologies for detection of DNA damage and mutations, edited by Pfeifer, G. P. Penum Press, New York: 1996.

Timmermans, P., Wong, P., Chiu, A., Herblin, W., Benfield, P., Carini, D., Lee, R., Wexler, R., Saye, J. and Smith, R. Angiotensin II receptor and angiotensin II receptor antagonists. Pharmacol Rev. 1993; 45:205-251.

Tsuchida, S., Matsusaka T., Chen X., Okubo S., Niimura F., Nishimura H., Fogo A., Utsunomiya H., Inagami T. and Ichikawa I. (1998) Murine double nullizygotes of the angiotensin type 1A and 1B receptor genes duplicate severe abnormal phenotypes of angiotensinogen nullizygotes. J Clin Invest, 101, 755-60

U.S. Pat. No. 3,832,337

U.S. Pat. No. 4,191,753

U.S. Pat. No. 4,512,979

U.S. Pat. No. 4,585,758

U.S. Pat. No. 4,680,283

U.S. Pat. No. 4,692,459

U.S. Pat. No. 5,071,955

U.S. Pat. No. 5,449,661

U.S. Pat. No. 5,556,780

U.S. Pat. No. 5,922,688

Uematsu, F., Kikuchi, H., Motomiya, M., Abe, T., Ishioka, C., Kanamaru, R., Sagami, I. and Watanabe, M. Human cytochrome P450IIE1 gene: Dral polymorphism and susceptibility to cancer. Tohoku J Exp Med. 1992; 168:113-117.

Varela, A. S. and Bosco Lopez Saez, J. J. Utility of serum activity of angiotensin-converting enzyme as a tumor marker. Oncology. 1993; 50:430-435.

Viswanathan, M. and Saavedra J. M. (1992) Expression of angiotensin II AT2 receptors in the rat skin during experimental wound healing. Peptides, 13, 783-6.

Vogel, V. G. and McPherson, R. S. Dietary epidemiology of colon cancer. Hematol Oncol Clin North Am. 1989; 3:35-63.

Volpert, O. V., Ward W. F., Lingen M. W., Chesler L., Solt D. B., Johnson M. D., Molteni A., Polverini P. J. and Bouck N. P. (1996) Captopril inhibits angiogenesis and slows the growth of experimental tumors in rats. J Clin Invest, 98, 671-679.

Wali, R. K., Skarosi S., Hart J., Zhang Y., Dolan M. E., Moschel R. C., Nguyen L., Mustafi R., Brasitus T. A. and Bissonnette M. (1999) Inhibition of O(6)-methylguanine-DNA methyltransferase increases azoxymethane-induced colonic tumors in rats. Carcinogenesis, 20, 2355-60.

Wang, H., Lu, D., Reaves, P. Y., Katovich, M. J. and Raizada, M. K. Retrovirally mediated delivery of angiotensin II type 1 receptor antisense in vitro and in vivo. Methods Enzymol. 2000; 314:581-590.

Watanabe, J., Hayashi, S. and Kawajiri, K. Different regulation and expression of the human CYP2E1 gene due to the Rsal polymorphism in the 5′-flanking region. J Biochem (Tokyo). 1994; 116:321-6.

Weston, A. and Harris C. C. (1997) Chemical Carcinogenesis. In Holland, J. F., Bast, R. C., Jr, Morton, D. L., Frei, E., III, Kufe, D. W. and Weichselbaum, R. R. (eds), Cancer Medicine. (Fourth edn). Vol. One. Williams & Wilkins, Baltimore, Md., pp. 261-276.

Whitebread, S., Mele, M., Kamber, B. and de Gasparo, M. Preliminary biochemical characterization of two angiotensin II receptor subtypes. Biochem Biophys Res Commun. 1989; 163:284-291.

Whitehead, R. H. and Joseph, J. L. Derivation of conditionally immortalized cell lines containing the Min mutation from the normal colonic mucosa and other tissues of an “Immortomouse”/Min hybrid. Epithelial Cell Biol. 1994; 3:119-25.

Whitlock, J. P. and Denison, M. S. Induction of cytochrome P450 enzymes that metabolize xenobiotics. In: Cytochrome P450: Structure, Mechanism and Biochemistry (Second ed.), edited by Montellano, P. R. O. d. Plenum Press, New York: 1995.

Whitlock, J. P., Jr. Induction of cytochrome P4501A1. Annu Rev Pharmacol Toxicol. 1999; 39:103-25.

Woodcroft, K. J. and Novak, R. F. Xenobiotic-enhanced expression of cytochromes P450 2E1 and 2B in primary cultured rat hepatocytes. Drug Metab Dispos. 1998; 26:372-378.

Xi, X. P., Graf K., Goetze S., Fleck E., Hsueh W. A. and Law R. E. (1999) Central role of the MAPK pathway in Ang II-mediated DNA synthesis and migration in rat vascular smooth muscle cells. Arterioscler Thromb Vasc Biol, 19, 73-82.

Xu, L., Li, A. P., Kaminski, D. L. and Ruh, M. F. 2,3,7,8 Tetrachlorodibenzo-p-dioxin induction of cytochrome P4501A in cultured rat and human hepatocytes. Chem Biol Interact. 2000; 124:173-89.

Yamada, T., Horiuchi, M. and Dzau, V. J. Angiotensin II type 2 receptor mediates programmed cell death. Proc Natl Acad Sci USA. 1996; 93:156-60.

Yang, C. S., Yoo, J. S., Ishizaki, H. and Hong, J. Y. Cytochrome P450IIE1: roles in nitrosamine metabolism and mechanisms of regulation. Drug Metab Rev. 1990; 22:147-159.

Yoshiji, H., Kuriyama S., Kawata M., Yoshii J., Ikenaka Y., Noguchi R., Nakatani T., Tsujinoue H. and Fukui H. (2001) The angiotensin-l-converting enzyme inhibitor perindopril suppresses tumor growth and angiogenesis: possible role of the vascular endothelial growth factor. Clin Cancer Res, 7, 1073-8.

Zafari, A. M., Ushio-Fukai, M., Akers, M., Yin, Q., Shah, A., Harrison, D. G., Taylor, W. R. and Griendling, K. K. Role of NADH/NADPH oxidase-derived H2O2 in angiotensin 11-induced vascular hypertrophy. Hypertension. 1998; 32:488-95.

Zhu, M., Gelband, C. H., Moore, J. M., Posner, P. and Sumners, C. Angiotensin II type 2 receptor stimulation of neuronal delayed-rectifier potassium current involves phospholipase A2 and arachidonic acid. J Neurosci. 1998; 18:679-686.

It will be understood that various details of the invention can be changed without departing from the scope of the invention. Moreover, it is not the inventor's desire to be bound by theory or mechanism. Any theory or mechanism presented herein is included solely to supplement the disclosure, and should not be interpreted to impose any limitation on the claims presented hereinbelow. Therefore, the foregoing description is for the purpose of illustration only, and not for the purpose of limitation-the invention being defined by the claims.

What is claimed is:

1. A method of decreasing a biological function of an AT₂ receptor in a subject in need thereof, comprising administering an effective amount of an therapeutic agent to the subject to decrease a biological function of an AT₂ receptor.

2. The method of claim 1, wherein the subject is a mammal.

3. The method of claim 2, wherein the mammal is a human.

4. The method of claim 2, wherein the mammal is a mouse.

5. The method of claim 1, wherein the therapeutic agent is PD 123319.

6. The method of claim 1, wherein the administration is orally, intramuscularly, subcutaneously, intraperitoneally, intravenously, intradermally or mucosally.

7. A method of preventing the development of a cancer in a subject having an AT₂ receptor, the method comprising:
(a) providing a subject having an AT₂ receptor; and
(b) administering to the subject a therapeutically effective amount of an AT₂ receptor antagonist, whereby the development of a cancer in a subject having an AT₂ receptor is prevented.

8. The method of claim 7, wherein the subject is a mammal.

9. The method of claim 8, wherein the mammal is a human.

10. The method of claim 8, wherein the mammal is a mouse.

11. The method of claim 7, wherein the cancer is a colorectal cancer.

12. The method of claim 7, wherein AT₂ receptor antagonist is PD 123319.

13. The method of claim 7, wherein the administration is orally, intramuscularly, subcutaneously, intraperitoneally, intravenously, intradermally or mucosally.

14. A method of treating a cancer in a subject having an AT₂ receptor, the method comprising:
(a) providing a subject having a cancer and an AT₂ receptor;
(b) administering to the subject a therapeutically effective amount of an AT₂ receptor antagonist, whereby a cancer in a subject having an AT₂ receptor is treated.

15. The method of claim 14, wherein the subject is a mammal.

16. The method of claim 15, wherein the mammal is a human.

17. The method of claim 15, wherein the mammal is a mouse.

18. The method of claim 14, wherein AT₂ receptor antagonist is PD 123319.

19. The method of claim 14, wherein the cancer is a colorectal cancer.

20. The method of claim 14, wherein the administration is orally, intramuscularly, subcutaneously, intraperitoneally, intravenously, intradermally or mucosally.

21. A method of screening one or more candidate compounds for an ability to prevent or treat a cancer, the method comprising:
(a) providing one or more candidate compounds;
(b) determining a first level of AT₂ receptor biological activity;
(c) contacting one candidate compound with an AT₂ receptor;
(d) determining a second level of AT₂ receptor biological activity;
(e) comparing the first level of AT₂ biological activity with the second level of AT₂ biological activity, wherein a second level that is less than the first level is indicative of a compound having an ability to prevent or treat a cancer; and
(f) repeating steps (b)-(e) for each of the one or more candidate compounds.

22. The method of claim 21, wherein the subject is a mammal.

22. The method of claim 22, wherein the mammal is a human.

24. The method of claim 22, wherein the mammal is a mouse.

25. The method of claim 21, wherein AT₂ receptor antagonist is PD 123319.

26. The method of claim 21, wherein the cancer is a colorectal cancer.

27. The method of claim 21, wherein the determining is by employing a technique selected from the group consisting of spectrophotometric detection, radiologic detection and immunologic detection.

28. A method achieving one of treating a cancer and preventing a cancer, the method comprising:
(a) providing subject in need of treatment;
(b) administering an ACE inhibitor to the subject; and
(c) administering an Ang II receptor antagonist to a subject.

29. The method of claim 28, wherein the male subject is a mammal.

30. The method of claim 29, wherein the male subject is a human.

31. The method of claim 29, wherein the male subject is a mouse.

32. The method of claim 28, wherein the ACE inhibitor and the Ang II receptor antagonist are coadministered contemporaneously.

33. The method of claim 32, wherein the coadministering is by injection.

34. The method of claim 28, wherein the cancer is a colorectal cancer.

35. The method of claim 28, wherein the ACE inhibitor is selected from the group consisting of Captopril, Enalapril, Lisinopril, Benazepril, Quinapril, Ramipril, Trandolapril, Moexipril, Fosinopril, Perindep and pharmaceutically acceptable salts thereof.

36. The method of claim 28, wherein the Ang II receptor antagonist is selected from the group consisting of candesartan cilexetil, eprosartan, irbesartan, tasosartan, telmisartan, valsartan, BMS-184699, 3-(2′-(tetrazol-5-yl)-1,1′-biphen-4-yl)methyl-5,7-dimethyl-2-ethyl-3H-imidazo[4,5-b]pyridine, BAY 106734, BIBR363, CL329167, E4177, EMD73495, HN65021, HR720, HOE720, LRB081, SC52459, SL910102, UP2696, YM358, EMD66397, ME3221, TAK536, BMS 184698, CGP42112A, CGP49870, CP14R130, E4188, EMD666R4, EXP9954, FRI 153332, GA0050, KT3579, LF70156, LRB057, LY266099, LY301875, PD 123177, PD 126055, SC51757, SC54629, U96849, UK77778, WAY126227, WK1260, WK1492, YH1498, and YM31472, PD 123319, Saralasin, and Losartan.


SEQUENCE LISTING (12 sequences)

SEQ ID NO: 1 - DNA, 1092 bp, Homo sapiens
  CDS (1)..(1092)
atgaagggcaactccacccttgccactactagcaaaaacattaccagcggtcttcacttcgggcttgtgaacatctctggcaacaatgagtctaccttgaactgttcacagaaaccatcagataagcatttagatgcaattcctattctttactacattatatttgtaattggatttctggtcaatattgtcgtggttacactgttttgttgtcaaaagggtcctaaaaaggtttctagcatatacatcttcaacctcgctgtggctgatttactccttttggctactcttcctctatgggcaacctattattcttatagatatgactggctctttggacctgtgatgtgcaaagtttttggttcttttcttaccctgaacatgtttgcaagcattttttttatcacctgcatgagtgttgataggtaccaatctgtcatctacccctttctgtctcaaagaagaaatccctggcaagcatcttatatagttccccttgtttggtgtatggcctgtttgtcctcattgccaacattttattttcgagacgtcagaaccattgaatacttaggagtgaatgcttgcattatggctttcccacctgagaaatatgcccaatggtcagctgggattgccttaatgaaaaatatccttggttttattatccctttaatattcatagcaacatgctattttggaattagaaaacacttactgaagacgaatagctatgggaagaacaggataacccgtgaccaagtcctgaagatggcagctgctgttgttctggccttcatcatttgctggcttcccttccatgttctgaccttcctggatgctctggcctggatgggtgtcattaatagctgcgaagttatagcagtcattgacctggcacttccttttgccatcctcttgggattcaccaacagctgcgttaatccgtttctgtattgttttgttggaaaccggttccaacagaagctccgcagtgtgtttagggttccaattacttggctccaagggaaaagagagagtatgtcttgccggaaaagcagttctcttagagaaatggagacctttgtgtcttaa

SEQ ID NO: 2 - protein, 363 aa, Homo sapiens
MKGNSTLATTSKNITSGLHFGLVNISGNNESTLNCSQKPSDKHLDAIPILYYIIFVIGFLVNIVVVTLFCCQKGPKKVSSIYIFNLAVADLLLLATLPLWATYYSYRYDWLFGPVMCKVFGSFLTLNMFASIFFITCMSVDRYQSVIYPFLSQRRNPWQASYIVPLVWCMACLSSLPTFYFRDVRTIEYLGVNACIMAFPPEKYAQWSAGIALMKNILGFIIPLIFIATCYFGIRKHLLKTNSYGKNRITRDQVLKMAAAVVLAFIICWLPFHVLTFLDALAWMGVINSCEVIAVIDLALPFAILLGFTNSCVNPFLYCFVGNRFQQKLRSVFRVPITWLQGKRESMSCRKSSSLREMETFVS

SEQ ID NO: 3 - DNA, 1092 bp, Mus musculus
  CDS (1)..(1092)
atgaaggacaacttcagttttgctgccaccagcagaaacattaccagcagccgtccttttgataatctcaacgcaactggcaccaatgagtccgcctttaattgctcacacaaaccatcagataagcatttggaagcaattcctgttctctactacatgatttttgtgattgggtttgctgttaatattgttgtggtctcactgttttgttgtcaaaagggccctaaaaaggtgtccagcatttacatcttcaatctggccttggctgacttactccttttggctaccctccctctctgggcaacctattactcttatagatatgattggctttttggacctgtgatgtgcaaagtgtttggttcttttctgactctgaacatgtttgcaagcattttttttattacctgcatgagtgtcgataggtaccaatcggtcatctacccttttctgtctcaaagaaggaatccctggcaagcatcttatgtagttccccttgtttggtgtatggcttgtctatcctcattgccaacattttatttccgggatgtcagaaccattgaatacttaggtgtgaatgcttgtattatggctttcccacccgagaaatatgctcagtggtctgctgggattgccttaatgaaaaatattcttggctttattattcctttaatattcatagcaacgtgttactttggaatcagaaaacatctgctgaagactaatagctatgggaagaacagaattacccgtgaccaagtcctgaagatggcagctgctgttgtgttggcattcatcatttgctggcttcccttccatgttctgaccttcttggatgctctgacctggatgggtatcattaatagctgtgaagttatagcagtcattgacctggcacttccttttgccatcctcctgggattcaccaacagctgtgttaatcccttcctgtattgttttgttggaaaccgcttccaacagaagctccgcagtgtgtttagagttcccattacttggctccaaggcaagagagagactatgtcttgcagaaaaggcagttctcttagagaaatggacacctttgtgtcttaa

SEQ ID NO: 4 - protein, 363 aa, Mus musculus
MKDNFSFAATSRNITSSRPFDNLNATGTNESAFNCSHKPSDKHLEAIPVLYYMIFVIGFAVNIVVVSLFCCQKGPKKVSSIYIFNLALADLLLLATLPLWATYYSYRYDWLFGPVMCKVFGSFLTLNMFASIFFITCMSVDRYQSVIYPFLSQRRNPWQASYVVPLVWCMACLSSLPTFYFRDVRTIEYLGVNACIMAFPPEKYAQWSAGIALMKNILGFIIPLIFIATCYFGIRKHLLKTNSYGKNRITRDQVLKMAAAVVLAFIICWLPFHVLTFLDALTWMGIINSCEVIAVIDLALPFAILLGFTNSCVNPFLYCFVGNRFQQKLRSVFRVPITWLQGKRETMSCRKGSSLREMDTFVS

SEQ ID NO: 5 - protein, 10 aa, Homo sapiens
DRVYIHPFHL

SEQ ID NO: 6 - protein, 8 aa, Homo sapiens
DRVYIHPF

SEQ ID NO: 7 - protein, 8 aa, Artificial Sequence
  Synthesized compound comprising sarcosine and a peptide
XRVYVHPA

SEQ ID NO: 8 - protein, 15 aa, Mus musculus
MKDNFSFAATSRNIT

SEQ ID NO: 9 - protein, 24 aa, Mus musculus
GIRKHLLKTNSYGKNRITRDQVLK

SEQ ID NO: 10 - protein, 15 aa, Mus musculus
QQKLRSVFRVPITWL

SEQ ID NO: 11 - DNA, 33 bp, Artificial Sequence
  Sense primer for PCR
ccaagcttgcttcagcctgcattttaaggagtg

SEQ ID NO: 12 - DNA, 33 bp, Artificial Sequence
  Antisense primer for PCR
ccaagcttgtgcatcccactaacagatttaaga